SEQUENCE LISTING (383 sequences)

SEQ ID NO: 1 - DNA, 705 bp, Homo sapiens
  source 1..705: mol_type = other DNA
atggaaaccccggcgcagctgctgtttctgctgctgctgtggctgccggataccaccggcgaaattgtgttgacgcagtctccaggcaccctgtctttgtctccaggggacagagccaccctctcctgcagggccagtcagagtgttagtagcagctactttgcctggtaccagcagaaacctggccaggctcccaggctcctcatttatggtgcatccagtagggccactggcatcccagacaggttcagtggcagtgggtctgggacagacttcactctcaccatcagcagactggagcctgaagattttgcagtgtattactgtcagcagtatgataggtcacctcggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - protein, 235 aa, Homo sapiens
METPAQLLFLLLLWLPDTTGEIVLTQSPGTLSLSPGDRATLSCRASQSVSSSYFAWYQQKPGQAPRLLIYGASSRATGIPDRFSGSGSGTDFTLTISRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1389 bp, Homo sapiens
  source 1..1389: mol_type = other DNA
atggattggacctggcgtattctgtttctggtggcggcggcgaccggcgcgcatagccaggtgcagctggtgcagtctggggctgaggtgaagaagcctggggcctcagtgaaggtctcctgtaagtcttctggatacaccttcaccggctactatatgcactgggtgcgacaggcccctggacaagggcttgagtggatgggatggatcaaccctaacagtggtggcacaaactatgcacagaagtttaagggcagggtcaccatgaccagggacacgtccatcagcacagcctacatggagctgagcaggctgagatctgacgacacggccgtgtattactgtgcgagagataagtggctggacggctttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - protein, 463 aa, Homo sapiens
MDWTWRILFLVAAATGAHSQVQLVQSGAEVKKPGASVKVSCKSSGYTFTGYYMHWVRQAPGQGLEWMGWINPNSGGTNYAQKFKGRVTMTRDTSISTAYMELSRLRSDDTAVYYCARDKWLDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - DNA, 726 bp, Homo sapiens
  source 1..726: mol_type = other DNA
atggacatgagggtgcccgctcagctcctggggctcctgctgctgtggctgagaggtgcgcgctgtgatattgtgatgacccagactccactctccctgcccgtcacccctggagagccggcctccatctcctgcaggtctagtcagagcctcttgaatagtgttgatggaagcaccaatttggactggtatctgcagaagccagggcagtctccacagctcctgatctatacgctttcctatcgggcctctggagtcccagacaggttcagtggcagtgggtcaggcactgacttcacactgaaaatcagcagggtggaggctgaggatgttggagtttattactgcatgcaacgtatagagtttccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - protein, 242 aa, Homo sapiens
MDMRVPAQLLGLLLLWLRGARCDIVMTQTPLSLPVTPGEPASISCRSSQSLLNSVDGSTNLDWYLQKPGQSPQLLIYTLSYRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 1395 bp, Homo sapiens
  source 1..1395: mol_type = other DNA
atggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtggtagttccatatactacgcagactctgtgaagggccgattcaccatctccagagacaatgccaagaactcactgtatctgcaaatgaacagcctgagagacgaggacacggctgtgtattactgtgcgagagagaggtactacggtgacacgccctttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 465 aa, Homo sapiens
MELGLCWVFLVAILEGVQCEVQLVESGGGLVQPGGSLRLSCAASGFTFSSYSMNWVRQAPGKGLEWVSYISSSGSSIYYADSVKGRFTISRDNAKNSLYLQMNSLRDEDTAVYYCARERYYGDTPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - DNA, 699 bp, Homo sapiens
  source 1..699: mol_type = other DNA
atgtcgccatcacaactcattgggtttctgctgctctgggttccagcctccaggggtgaaattgtgctgactcagtctccagactttcagtctgtgactccaaaggagaaagtcaccatcacctgccgggccagtcagagcattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcagcgtattactgtcatcagagtagtagtttacctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 233 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1389 bp, Homo sapiens
  source 1..1389: mol_type = other DNA
atggggtcaaccgccatcctcgccctcctcctggctgttctccaaggagtctgtgccgaggtgcagctgatgcagtctggagcagaggtgaaaaagcccggggagtctctgaagatctcctgtaagggttctggatacagcttttccttccactggatcgcctgggtgcgccagatgcccgggaaaggcctggagtggatggggatcatccatcctggtgcctctgataccagatacagcccgtccttccaaggccaggtcaccatctcagccgacaactccaacagcgccacctacctgcagtggagcagcctgaaggcctcggacaccgccatgtatttctgtgcgagacaaagggaactcgactactttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 463 aa, Homo sapiens
MGSTAILALLLAVLQGVCAEVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - DNA, 708 bp, Homo sapiens
  source 1..708: mol_type = other DNA
atggacatgagggtccccgctcagctcctggggctcctgctgctgtggctgagaggtgcgcgctgtgatatccagatgacccaatccccttctagtctgtccgcttctgtgggcgacagggttacaataacttgcaaagcgagccaggacatcaacaaatatgtggcttggtatcagcaaaaacccggcaaggcaccaaaattgctcatttattacacgtcatggctccagcctggtgtacctagcaggttttccggctccggctcaggtaccgactttactttcactatctcctcactgcagccggaggacattgccacatactactgtctccaatatgataacttgttgtatacttttgggcaaggaactaagctcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 236 aa, Homo sapiens
MDMRVPAQLLGLLLLWLRGARCDIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1401 bp, Homo sapiens
  source 1..1401: mol_type = other DNA
atggacatgagggtgcccgctcagctcctggggctcctgctgctgtggctgagaggtgcgcgctgtgaggttcagcttgtggagtctggcggcggcctggtgcagccaggcggttcactcaggctgagctgtgctgcatcagggttcaccttcagccgttactggatgaactgggtgcggcaagcacccgggaaaggcctggagtgggtggctcagattcgcttgaaaagtgacaattatgccactcactatgcagaaagcgtgaaggggcgctttacaatttctagagacaacgccaaaaactcactgtacctgcagatgaacagcctcagagctgaggatacagctgtgtattattgtactgaggggctcgactattggggacagggcacgacagtgaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 467 aa, Homo sapiens
MDMRVPAQLLGLLLLWLRGARCEVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 708 bp, Homo sapiens
  source 1..708: mol_type = other DNA
atggacatgagggtcctcgctcagctcctggggctcctgctgctctgtttcccaggtgccagatgtgacatccagatgacccagtctccatcctcactgtctgcatctgtaggagacagagtcaccatcacttgtcgggcgagtcagggtattagcaactggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtatgatagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - protein, 236 aa, Homo sapiens
MDMRVLAQLLGLLLLCFPGARCDIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1398 bp, Homo sapiens
  source 1..1398: mol_type = other DNA
atggagttggggctgaactgggttttccttgttgctattttagaaggtgtccactgtgaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 466 aa, Homo sapiens
MELGLNWVFLVAILEGVHCEVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - DNA, 723 bp, Homo sapiens
  source 1..723: mol_type = other DNA
atggacatgaggctccctgctcagctcctggggctgctaatgctctgggtcccaggatccagtggggatgttctgatgactcagtctccactctccctgcccgtcacccttggacagccggcctccatctcctgcaggtctagtcaaagcatcgtacacagtaacggaaacacctacttggagtggtatctgcagaggccaggccaatctccaaagctcctaatttataaggtttctaaccggttctctggggtcccagacagattcagcggcagtgggtcaggcactgatttcacactgaaaatcagcagggtggaggctgaggatgttggggtttattactgcttccaaggtagccacgttcctctgacgttcggcgcagggaccaagctggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 22 - protein, 241 aa, Homo sapiens
MDMRLPAQLLGLLMLWVPGSSGDVLMTQSPLSLPVTLGQPASISCRSSQSIVHSNGNTYLEWYLQRPGQSPKLLIYKVSNRFSGVPDRFSGSGSGTDFTLKISRVEAEDVGVYYCFQGSHVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1410 bp, Homo sapiens
  source 1..1410: mol_type = other DNA
atggacacactttgctccacgctcctgctgctgaccatcccttcatgggtcttgtcccaggtcaccttgaaggagtctggtcctgcgctggtgaaacccacacagaccctcacgctgacctgcaccttctctgggttctcactccgcactagtggaatgggcgtgggctggatccgtcagcccccaggaaaggccctggagtggcttgcccacatttggtgggatgatgataagagctacaacccatctctgaagagccagctcaccatctctaaggacacctccaaaaaccaggtggtccttacaatgaccaacatggaccctgtggacacagccacatattactgtgcacgcagaaactattactacgacgactacttcgcctactggggccagggcaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 470 aa, Homo sapiens
MDTLCSTLLLLTIPSWVLSQVTLKESGPALVKPTQTLTLTCTFSGFSLRTSGMGVGWIRQPPGKALEWLAHIWWDDDKSYNPSLKSQLTISKDTSKNQVVLTMTNMDPVDTATYYCARRNYYYDDYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 705 bp, Homo sapiens
  source 1..705: mol_type = other DNA
atggaaaccccggcgcagctgctgtttctgctgctgctgtggctgccggataccaccggcgaaattgtgttgacgcagtctccaggcaccctgtctttgtctccaggggaaagagccaccctctcctgcagggccagtcagagtgttagcagcagctacttcgcctggtaccaacagaaacctggccaggctcccaggctcctcatctatggtacatccagcagggccactggcatcccagacaggttcagtggcagtgggtctgggacagacttcactctcaccgtcagcagactggagcctgaggattttgcagtgtattactgtcagcagtatgataggtcacctcggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 26 - protein, 235 aa, Homo sapiens
METPAQLLFLLLLWLPDTTGEIVLTQSPGTLSLSPGERATLSCRASQSVSSSYFAWYQQKPGQAPRLLIYGTSSRATGIPDRFSGSGSGTDFTLTVSRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 1389 bp, Homo sapiens
  source 1..1389: mol_type = other DNA
atggattggacctggcgtattctgtttctggtggcggcggcgaccggcgcgcatagccaggtgcagttggtgcagtctggggctgcggtgaagaagcctggggcctcagtgaaggtctcctgcaaggcttctggatacaccttcaccggctactatatacactgggtgcgacaggcccctggacaagggcttgagtggatgggatggatcaaccctaacagtggtggcacaaactatgcacaaaagtttcagggcagggtcaccatgaccagggacacgtccatcagcacagcctccatggaactgagcaggctgagatctgacgacacggccgtttatttctgtgcgagagatcggtggctggatgcttttgatatctggggccaagggacaatggtcaccgtctcttctgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 28 - protein, 463 aa, Homo sapiens
MDWTWRILFLVAAATGAHSQVQLVQSGAAVKKPGASVKVSCKASGYTFTGYYIHWVRQAPGQGLEWMGWINPNSGGTNYAQKFQGRVTMTRDTSISTASMELSRLRSDDTAVYFCARDRWLDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - DNA, 705 bp, Homo sapiens
  source 1..705: mol_type = other DNA
atggaaaccccagctcagcttctcttcctcctgctactctggctcccagataccaccggagaaattgtgttgacgcagtctccaggcaccctgtctttgtctccaggggaaagagccaccctctcctgcagggccagtcagagtgttagcagcggctacttaacctggtaccagcagaaacctggccaggctcccagactcctcatctatggtgcatccagcagggccactggcatcccagacaggttcagtggcagtgggtctgggacggacttcactctcaccatcagcagactggagcctgaagattttgcagtgtattactgtcagcagtatggtaactcactgagcaggtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 30 - protein, 235 aa, Homo sapiens
METPAQLLFLLLLWLPDTTGEIVLTQSPGTLSLSPGERATLSCRASQSVSSGYLTWYQQKPGQAPRLLIYGASSRATGIPDRFSGSGSGTDFTLTISRLEPEDFAVYYCQQYGNSLSRFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 1422 bp, Homo sapiens
  source 1..1422: mol_type = other DNA
atggacatgagggtgcccgctcagctcctggggctcctgctgctgtggctgagaggtgcgcgctgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcatcatctccagagataaatccaagaacacgctgtatctgcaaatgaacagcctgagagccgaggacacggctgtgtattactgtgcgagagcggggggtatagcagcagctggcctctactactactacggtatggacgtctggggccaagggaccacggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 32 - protein, 474 aa, Homo sapiens
MDMRVPAQLLGLLLLWLRGARCQVQLVESGGGVVQPGRSLRLSCAASGFTFSSYGMHWVRQAPGKGLEWVAVIWYDGSNKYYADSVKGRFIISRDKSKNTLYLQMNSLRAEDTAVYYCARAGGIAAAGLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - DNA, 806 bp, Homo sapiens
  source 1..806: mol_type = other DNA
atgacatccactttgcctttctctccacaggtgtccactcccaggtccaagtttaaacggatctctagcgaattccctctagagtcgactagaccaccatggacatgagggtgcccgctcagctcctggggctcctgctgctgtggctgagaggtgcgcgctgttcttctgagctgactcaggaccctactgtgtctgtggccttgggacagacagtcaaaatcacatgccaaggagacagcctcagaagtttttatgcaagctggtaccagcagaagccaggacaggcccctgtacttgtcttctatggtaaaaacaaccggccctcagggatcccagaccgattctctggctccagctcaggaaacacagcttccttgaccatcactggggctcaggcggaagatgaggctgactattattgtaattcccgggacagcagtgtttaccatctggtactcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 34 - protein, 236 aa, Homo sapiens
MDMRVPAQLLGLLLLWLRGARCSSELTQDPTVSVALGQTVKITCQGDSLRSFYASWYQQKPGQAPVLVFYGKNNRPSGIPDRFSGSSSGNTASLTITGAQAEDEADYYCNSRDSSVYHLVLGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 35 - DNA, 1434 bp, Homo sapiens
  source 1..1434: mol_type = other DNA
atggacatgagggtgcccgctcagctcctggggctcctgctgctgtggctgagaggtgcgcgctgtcaggtgcagttggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtatttctgtgcgagagatcaaatgagtattattatgcttcggggagtttttcccccttactattacggtatggacgtctggggccaagggaccacggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 478 aa, Homo sapiens
MDMRVPAQLLGLLLLWLRGARCQVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARDQMSIIMLRGVFPPYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - DNA, 723 bp, Homo sapiens
  source 1..723: mol_type = other DNA
atggacatgagggtgcccgctcagctcctggggctcctgctgctgtggctgagaggtgccagatgtgatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtagagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 38 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 1425 bp, Homo sapiens
  source 1..1425: mol_type = other DNA
atggacatgagggtgcccgctcagctcctggggctcctgctgctgtggctgagaggtgccagatgtgaggtgcagctggtggagtctgggggaggcttggtccagcctggggggtccctgagactctcctgtgcagcctccggattcacctttagtagctattggatgagctgggtccgccaggctccagggaaggggctggagtgggtggccagcataaaacaagatggaagtgagaaatactatgtggactctgtgaagggccgattcaccatctccagagacaacgccaggaactcactgtatctgcaaatgaacagcctgagagccgaggacacggctgtgtattactgtgcgagagatcttgtattaatggtgtatgatatagactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 475 aa, Homo sapiens
MDMRVPAQLLGLLLLWLRGARCEVQLVESGGGLVQPGGSLRLSCAASGFTFSSYWMSWVRQAPGKGLEWVASIKQDGSEKYYVDSVKGRFTISRDNARNSLYLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - DNA, 723 bp, Homo sapiens
  source 1..723: mol_type = other DNA
atggacatgagggtgcccgctcagctcctggggctcctgctgctgtggctgagaggtgccagatgtgatattgtgatgactcagtctccactctccctgcccgtcacccctggagagccggcctccatctcctgcaggtctagtcagagcctcctgcatagtaatgggtacaactatttggattggtacctgcagaagccagggcagtctccacagctcctgatctatttgggttctaatcgggcctccggggtccctgacaggttcagtggcagtggatcaggcacacatcttacactgaaaatcagcagagtggaggctgaggatgttggagtttattactgcatgcaaactctacaaactccgctcactttcggcggagggaccaaggtagagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTHLTLKISRVEAEDVGVYYCMQTLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 1425 bp, Homo sapiens
  source 1..1425: mol_type = other DNA
atggacatgagggtgcccgctcagctcctggggctcctgctgctgtggctgagaggtgccagatgtgaggtgcagctggtggagtctgggggaggcttggtccagcctggggggtccctgagactctcctgtgcagcctccggattcacctttagtaactattggatgagctgggtccgccaggctccagggaaggggctggagtgggtggccagcataaaacaagatggaagtgagaaatactatgtggactctgtgaagggccgattcgccatctccagagacaacgccaagaactcactgtttctgcaaatgaacagcctgagagccgaggacacggctgtgtattactgtgcgagagatcttgtactaatggtgtatgatatagactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 475 aa, Homo sapiens
MDMRVPAQLLGLLLLWLRGARCEVQLVESGGGLVQPGGSLRLSCAASGFTFSNYWMSWVRQAPGKGLEWVASIKQDGSEKYYVDSVKGRFAISRDNAKNSLFLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - DNA, 705 bp, Homo sapiens
  source 1..705: mol_type = other DNA
atgacttgttctccactgctgctgactctgctgattcattgtactggttcttgggcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcaaccaccctgggcatcaccggactccagactggggacgaggccgattattactgcggaacatgggatagccgcctgagtgctgtggttttcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 46 - protein, 235 aa, Homo sapiens
MTCSPLLLTLLIHCTGSWAQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 47 - DNA, 1425 bp, Homo sapiens
  source 1..1425: mol_type = other DNA
atggaatttggtctgtcttgggtatttctggttgctctgctgcgtggcgtgcagtgccaggtgcagctggtggaatctgggggaggcgtggtccagcctgggaggtccctgagactctcctgtgcagcctctggattcaccttcagtagctttggcatgcactgggtccgccaggctccaggcaaggggctggagtgggtggcagttatatcatttgatggaagtattaagtattctgtagactccgtgaagggccgattcaccatctccagagacaattcaaagaacacgctgtttctgcaaatgaacagcctgcgagccgaggacacggctgtgtattactgtgcgagagatcggctcaattactatgatagtagtggttattatcactacaaatactacggtatggccgtctggggccaagggaccacggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 475 aa, Homo sapiens
MEFGLSWVFLVALLRGVQCQVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 49 - DNA, 702 bp, Homo sapiens
  source 1..702: mol_type = other DNA
atggacatgagggtgcccgctcagctcctggggctcctgctgctgtggctgagaggtgccagatgttatgagctgactcagccaccctcagtgtccgtgtccccaggacagacagccaccatcacctgctctggagataaattgggggaaagatatgcgtcttggtatcagcagaggccaggccagtcccctgtactggtcatctatcaagatatcaagcggccctcagggatccctgagcgattctctggctccaactctgggaacacagccactctgaccatcagcgggacccaggctatggatgaggctgactatttctgtcaggcgtggtacagcagcaccaatgtgcttttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 50 - protein, 234 aa, Homo sapiens
MDMRVPAQLLGLLLLWLRGARCYELTQPPSVSVSPGQTATITCSGDKLGERYASWYQQRPGQSPVLVIYQDIKRPSGIPERFSGSNSGNTATLTISGTQAMDEADYFCQAWYSSTNVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 51 - DNA, 1398 bp, Homo sapiens
  source 1..1398: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgctgaaagtaataaatactacgcagactccgtgaagggccgattcaccatctccagagacaattccaagaacacgctgtatctgcaaatgaacagcctgagagccgaggacacggctgtgtattactgtgcgagagcccaggagggtatagcccctgacgcttttgatatctggggccaaggaacaatggtcaccgtctcttcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 52 - protein, 466 aa, Homo sapiens
MEFGLSWVFLVALLRGVQCQVQLVESGGGVVQPGRSLRLSCAASGFTFSSYGMHWVRQAPGKGLEWVAVIWYAESNKYYADSVKGRFTISRDNSKNTLYLQMNSLRAEDTAVYYCARAQEGIAPDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - DNA, 702 bp, Homo sapiens
  source 1..702: mol_type = other DNA
atggggtcaaccgccatccttggcctcctcctggctgtcctgcagggagggcgcgccgaaattgtgttgacgcagtctccaggcaccctgtctttgtctccaggggaaagagccaccctctcctgcagggccagtcagagtgttagcagcagctacttagcctggcaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 54 - protein, 234 aa, Homo sapiens
MGSTAILGLLLAVLQGGRAEIVLTQSPGTLSLSPGERATLSCRASQSVSSSYLAWHQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 1389 bp, Homo sapiens
  source 1..1389: mol_type = other DNA
atggggtcaaccgccatccttggcctcctcctggctgtcctgcagggagggcgcgccgaggtgcagctattggagtctgggggaggcttggtacagcctggggggtccctgagactctcctgtgcagcctctggattcacctttagcacctatgtcatgagctgggtccgccaggctccagggaaggggctggagtgggtctcaagtattagtggtagtggtcttggctcatactacgcagactccgtgaagggccggttcaccatctccagagacaattccaagaacacgctgtatctgcaaatgaacagcctgagagccgaggacacggccgtatattactgtgcgaaagaggcccatcgggggccctttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 56 - protein, 463 aa, Homo sapiens
MGSTAILGLLLAVLQGGRAEVQLLESGGGLVQPGGSLRLSCAASGFTFSTYVMSWVRQAPGKGLEWVSSISGSGLGSYYADSVKGRFTISRDNSKNTLYLQMNSLRAEDTAVYYCAKEAHRGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - DNA, 721 bp, Homo sapiens
  source 1..721: mol_type = other DNA
atgaggctccttgctcagcttctggggctgctaatgctctgggtccctggatccagtggggatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagaaatttaaccggttctctggggtcccagacagattcagtggcagtggggcagggacagatttcacactgaaaatcagcagggtggaagctgaggatgtcggggtttattactgcatgcaagctacacaaattcctctcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgag

SEQ ID NO: 58 - protein, 239 aa, Homo sapiens
MRLLAQLLGLLMLWVPGSSGDIVMTQTPLSSPVTLGQPASISCRSSQSLVHSDGNTYLSWLQQRPGQPPRLLIYKKFNRFSGVPDRFSGSGAGTDFTLKISRVEAEDVGVYYCMQATQIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1416 bp, Homo sapiens
  source 1..1416: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtttctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagggggttatgattacgtttgggggagttatcgccgtaactccgactttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - protein, 472 aa, Homo sapiens
MEFGLSWVFLVALLRGVQCQVQLVESGGGVVQPGRSLRLSCAASGFTFSFYGMHWVRQAPGKGLEWVAVIWYDGSNKYYADSVKGRFTISRDNSKNTLYLQMNSLRAEDTAVYYCARGGYDYVWGSYRRNSDFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - DNA, 705 bp, Homo sapiens
  source 1..705: mol_type = other DNA
atggaagccccagctcagcttctcttcctcctgctactctggctcccagataccactggagaaatagtgatgacgcagtctccagccaccctgtctgtgtctccaggggaaagagccaccctctcctgcagggccagtcagagtgttgacagcaacttagcctggtaccggcagaaacctggccaggctcccaggctcctcatctatggtgcatccaccagggccactggtatcccagccaggttcagtggcagtgggtctgggactgagttcactctcaccatcagcagcctgcagtctgaagattttgcagtttattactgtcagcagtatattaactggcctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 62 - protein, 235 aa, Homo sapiens
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 1395 bp, Homo sapiens
  source 1..1395: mol_type = other DNA
atgaaacacctgtggttcttccttctcctggtggcagctcccagatgggtcctgtcccaggtgcagctgcaggagtcgggcccaggactggtgaagccttcggagaccctgtccctcacctgcactgtctctggtggctccatcagtatttactactggagctggatccggcagcccccagggaagggactggagtggattgggtatgtctattacagtgggagcaccaattacaacccctccctcaagagtcgagtcaccatatcagtagacacgtccaagaaccagttctccctgaagctgaactctgtgaccgctgcggacacggccgtgtattactgtgcgagagggggatacgatttttggagtggttattttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 64 - protein, 465 aa, Homo sapiens
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - DNA, 723 bp, Homo sapiens
  source 1..723: mol_type = other DNA
atggtgctgcagacccaggtgtttattagcctgctgctgtggattagcggcgcgtatggcgacatcgtgctgacccagtctccagactccctggctgtgtctctgggcgagagggccaccatcaactgcaagtccagccagagtattttatacagctccagcaatgagaacttcttaacttggtaccagcagaaaccaggacagcctcctaagctgctcatttactgggcatctacccgggaatccggggtccctgaccgattcagtggcagcgggtctgggacagatttcactctcaccatcagcagcctgcagcctgaagatgtggcagtttattactgtcagcaatattttagtgtttttcggacgttcggccaagggaccagggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 66 - protein, 240 aa, Homo sapiens
MVLQTQVFISLLLWISGAYGDIVLTQSPDSLAVSLGERATINCKSSQSILYSSSNENFLTWYQQKPGQPPKLLIYWASTRESGVPDRFSGSGSGTDFTLTISSLQPEDVAVYYCQQYFSVFRTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 1395 bp, Homo sapiens
  source 1..1395: mol_type = other DNA
atggattggacctggcgtattctgtttctggtggcggcggcgaccggcgcgcatagccaggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaaccctaacagtggtggcacaaactctgcacagaagtttcggggcagggtcaccatgaccagggacacgtccatcagcacagcctacatggagctgagcaggctgagatctgacgacacggccgtgtattactgtgcgcgagagggtggatacagctatggttactttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - protein, 465 aa, Homo sapiens
MDWTWRILFLVAAATGAHSQVQLVQSGAEVKKPGASVKVSCKASGYTFTGYYIHWVRQAPGQGLEWMGWINPNSGGTNSAQKFRGRVTMTRDTSISTAYMELSRLRSDDTAVYYCAREGGYSYGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 69 - DNA, 708 bp, Homo sapiens
  source 1..708: mol_type = other DNA
atggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagtgtgcagcctgaagattttgtaacttattactgtctacagcataatagtaaccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - protein, 236 aa, Homo sapiens
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 1419 bp, Homo sapiens
  source 1..1419: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatgtggtatgatggaagtaataaagactatgtagactccgtgaagggccgattcaccatctccagagacaattccaagaacacgctgtatctgcaaatgaaccgcctgagagccgaggacacggctgtgtattactgtgcgagagaaaaagatcattacgacattttgactggttataactactactacggtctggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 72 - protein, 473 aa, Homo sapiens
MEFGLSWVFLVALLRGVQCQVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 73 - DNA, 705 bp, Homo sapiens
  source 1..705: mol_type = other DNA
atggaaaccccggcgcagctgctgtttctgctgctgctgtggctgccggataccaccggcgaaattgtgttgacgcagtctccaggcaccctgtctttgtctccaggggaaagagccaccctctcctgcagggccagtcagtatattagcaacacctatttagcctggttccagcagaaacctggccaggctcccaggctcctcatctatggtgcagccaccagggccactggcatcccagacaggttcagtggcagtgggtctgggacagacttcactttcaccatcagcagactggagcctgaagattttgcagtgtattactgtcagcagtatggtagctcaccgtggacgttcggccaagggaccacggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 74 - protein, 235 aa, Homo sapiens
METPAQLLFLLLLWLPDTTGEIVLTQSPGTLSLSPGERATLSCRASQYISNTYLAWFQQKPGQAPRLLIYGAATRATGIPDRFSGSGSGTDFTFTISRLEPEDFAVYYCQQYGSSPWTFGQGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 1389 bp, Homo sapiens
  source 1..1389: mol_type = other DNA
atggattggacctggcgtattctgtttctggtggcggcggcgaccggcgcgcatagccaggtgcagttggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaggtttcggggcagggtcaccatgaccagggacacgtccatcagcacagcctacatggaactgagcaggctgagatctgacgacacggccgtgtattactgtgcgagagccccgtatgactggacctttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 76 - protein, 463 aa, Homo sapiens
MDWTWRILFLVAAATGAHSQVQLVQSGAEVKKPGASVKVSCKASGYTFTGYYMHWVRQAPGQGLEWMGWINPNSGGTNYAQRFRGRVTMTRDTSISTAYMELSRLRSDDTAVYYCARAPYDWTFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 77 - DNA, 708 bp, Homo sapiens
  source 1..708: mol_type = other DNA
atggacatgagggtgcccgctcagctcctggggctcctgctgctgtggctgagaggtgcgcgctgtgacatccagatgacccagtctccatcttccgtgtctgcatctgtaggagacagagtcaccatcacttgtcgggcgagtcagggtattagcaactggttagcctggtatcagcagaaaccagggacagcccctaaactcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 78 - protein, 236 aa, Homo sapiens
MDMRVPAQLLGLLLLWLRGARCDIQMTQSPSSVSASVGDRVTITCRASQGISNWLAWYQQKPGTAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - DNA, 1407 bp, Homo sapiens
  source 1..1407: mol_type = other DNA
atggacatgagggtgcccgctcagctcctggggctcctgctgctgtggctgagaggtgcgcgctgtcaggtgcagttggtgcagtctgggactgaggtgaagaagcctggggcctcaatgaaggtttcctgcaaggcatctggatacaccttcaccagctattatatgcactgggtgcgacaggcccctggacaagggcttgagtggatgggaataatcaaccctagtggtgatagcacaagctacgcacagaagttccagggcagagtcaccatgaccagggacacgtccacgaacacagtctacatggagctgagcagcctgagatctgaggacacggccatgtattactgtgcgagagatgtagaggttcggggaatttctcactttgactactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 80 - protein, 469 aa, Homo sapiens
MDMRVPAQLLGLLLLWLRGARCQVQLVQSGTEVKKPGASMKVSCKASGYTFTSYYMHWVRQAPGQGLEWMGIINPSGDSTSYAQKFQGRVTMTRDTSTNTVYMELSSLRSEDTAMYYCARDVEVRGISHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 81 - DNA, 714 bp, Homo sapiens
  source 1..714: mol_type = other DNA
atgggatggagctgtatcatcctcttcttggtagcaacagctacaggtgtccactccgacatccagctgacccagtctccatcatctctgagcgcatctgttggagatagggtcactatgagctgtaagtccagtcaaagtgttttatacagtgcaaatcacaagaactacttggcctggtaccagcagaaaccagggaaagcacctaaactgctgatctactgggcatccactagggaatctggtgtcccttcgcgattctctggcagcggatctgggacagattttactttcaccatcagctctcttcaaccagaagacattgcaacatattattgtcaccaatacctctcctcgtggacgttcggtggagggaccaaggtgcagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 82 - protein, 238 aa, Homo sapiens
MGWSCIILFLVATATGVHSDIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - DNA, 1395 bp, Homo sapiens
  source 1..1395: mol_type = other DNA
atgggatggagctgtatcatcctcttcttggtagcaacagctacaggtgtccactcccaggtccagctggtccaatcaggggctgaagtcaagaaacctgggtcatcagtgaaggtctcctgcaaggcttctggctacacctttactagctactggctgcactgggtcaggcaggcacctggacagggtctggaatggattggatacattaatcctaggaatgattatactgagtacaatcagaacttcaaggacaaggccacaataactgcagacgaatccaccaatacagcctacatggagctgagcagcctgaggtctgaggacacggcattttatttttgtgcaagaagggatattactacgttctactggggccaaggcaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 84 - protein, 465 aa, Homo sapiens
MGWSCIILFLVATATGVHSQVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 705 bp, Homo sapiens
  source 1..705: mol_type = other DNA
atggaaaccccagctcagcttctcttcctcctgctactctggctcccagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagcagactggagcctgaagattttgcagtgtattactgtcagcggtctggtggctcatcattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 86 - protein, 235 aa, Homo sapiens
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 1398 bp, Homo sapiens
  source 1..1398: mol_type = other DNA
atggggtcaaccgccatcctcgccctcctcctggctgttctccaaggagtctgtgccgaggtgcagctggtacagtctggagcagaggtgaaaaagcccggggagtctctgaagatctcctgtaagggttctggatacaactttaccagctactggatcggctgggtgcgccagatgcccgggaaaggcctggagttgatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtggttcggggagctacttttacttcgatctctggggccgtggcaccctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 88 - protein, 466 aa, Homo sapiens
MGSTAILALLLAVLQGVCAEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 723 bp, Homo sapiens
  source 1..723: mol_type = other DNA
atggacatgagggtgcccgctcagctcctggggctcctgctgctgtggctgagaggtgcgcgctgtgatattgtgatgactcagtctccactctccctgcccgtcacccctggagagccggcctccatctcctgcaggtctagtcagagcctcctgcatagtcatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggtacacactggcctccgacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 90 - protein, 241 aa, Homo sapiens
MDMRVPAQLLGLLLLWLRGARCDIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 1410 bp, Homo sapiens
  source 1..1410: mol_type = other DNA
atggacatgagggtgcccgctcagctcctggggctcctgctgctgtggctgagaggtgcgcgctgtgaggtccagctggtgcagtctgggggaggcgtggtccagcctgggaggtccctgagactctcctgtgcagcgtctggattcaccttcagtagctatggcatgcactgggtccgccaggctccaggcaaggggctggagtgggtttcatacattagtagtagtggtagtaccatatactacgcagactctgtgaagggccgattcaccatctccagggacaacgccaagaactcactatatctgcaaatgaacagcctgagagccgaggacacggccgtgtattactgtgcgagagatctgttagattacgatctgttgactggttatggctactggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - protein, 470 aa, Homo sapiens
MDMRVPAQLLGLLLLWLRGARCEVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 93 - DNA, 705 bp, Homo sapiens
  source 1..705: mol_type = other DNA
atggaaaccccagcgcagcttctcttcctcctgctactctggctcccagataccaccggagaaattgtgttgacgcagtctccaggcaccctgtctttgtctccaggggaaagagccaccctctcctgcagggccagtcagggtattagtagaagctacttagcctggtaccagcagaaacctggccaggctcccagcctcctcatctatggtgcatccagcagggccactggcatcccagacaggttcagtggcagtgggtctgggacagacttcactctcaccatcagcagactggagcctgaagattttgcagtgtattactgtcaacaatttggtagttcaccgtggacgttcggccaagggaccaaggtggaaatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 94 - protein, 235 aa, Homo sapiens
METPAQLLFLLLLWLPDTTGEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 1413 bp, Homo sapiens
  source 1..1413: mol_type = other DNA
atgaagcatctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactactactatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 96 - protein, 471 aa, Homo sapiens
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 708 bp, Homo sapiens
  source 1..708: mol_type = other DNA
atggacatgagggtccctgctcagctcctggggctcctgctgctctggctctcaggtgccagatgtgacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 98 - protein, 236 aa, Homo sapiens
MDMRVPAQLLGLLLLWLSGARCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - DNA, 1392 bp, Homo sapiens
  source 1..1392: mol_type = other DNA
atgaaacacctgtggttcttcctcctcctggtggcagctcccagatgggtcctgtcccaggtacagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagtcgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcttcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 100 - protein, 464 aa, Homo sapiens
MKHLWFFLLLVAAPRWVLSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 101 - DNA, 708 bp, Homo sapiens
  source 1..708: mol_type = other DNA
atggaaaccccagcgcagcttctcttcctcctgctactctggctcccagataccgctagcgaaattgtgttgacgcagtctccaggcaccctgtctttgtctccaggggaaagagccaccctctcctgcagggccagtcagagtgttagcaacagctacttagcctggtaccagcagaaacctggccaggctcccaggctcctcatctatggtgcatccagcagggcccctggcatcccagacaggttcagtggcagtgggtctgggacagacttcactctcaccatcagcagactggagcctgaagattttgcagtgtattactgtcagcagtatgatcactcagcagggtggacgttcggccaagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 102 - protein, 236 aa, Homo sapiens
METPAQLLFLLLLWLPDTASEIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - DNA, 1680 bp, Homo sapiens
  source 1..1680: mol_type = other DNA
atggggtcaaccgccatccttggcctcctcctggctgttctccaaggagtcgctagcgaggttcagctggtggagtctgggggaggcttggtacagcctggggggtccctgagactctcctgtgcagcctctggattcaccttcagtagaaatgctatgttctgggttcgccaggctccaggaaaaggtctggagtgggtatcaggtattggtactggtggtgccacaagctatgcagactccgtgaagggccgattcaccatctccagagacaatgccaagaactccttgtatcttcaaatgaacagcctgagagccgaggacacggctgtgtattactgtgcaagagggaggtactacttcccgtggtggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgaccgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 104 - protein, 460 aa, Homo sapiens
MGSTAILGLLLAVLQGVASEVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 105 - DNA, 702 bp, Homo sapiens
  source 1..702: mol_type = other DNA
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatcccgcagggccactggcatcccagacaggttcagtggcagtgggtctgggacagacttcactctcaccatcagcagactggagcctgaagattttgcagtgtattactgtcagcggtatggtagctcacacacttttggccaggggaccaagctggagatcagccgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 106 - protein, 234 aa, Homo sapiens
METPAQLLFLLLLWLPDTTGEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - DNA, 1401 bp, Homo sapiens
  source 1..1401: mol_type = other DNA
atggggtcaaccgccatcctcgccctcctcctggctgttctccaaggagtctgtgccgaggtgcagctggtgcagtctggagcagaggtgaaaaagcccggggagtctctgaagatctcctgtaaggtttctggatacttctttaccacctactggatcggctgggtgcgccagatgcccgggaaaggcctggagtatatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagagggggtaactggaactgctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 108 - protein, 467 aa, Homo sapiens
MGSTAILALLLAVLQGVCAEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 723 bp, Homo sapiens
  source 1..723: mol_type = other DNA
atggacatgagggtgcccgctcagctcctggggctcctgctgctgtggctgagaggtgccagatgtgatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattactgcatgcaaggtacacactggcctctgacgttcggccaagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 110 - protein, 241 aa, Homo sapiens
MDMRVPAQLLGLLLLWLRGARCDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - DNA, 1413 bp, Homo sapiens
  source 1..1413: mol_type = other DNA
atggacatgagggtgcccgctcagctcctggggctcctgctgctgtggctgagaggtgccagatgtcaggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagatggaccgggcgtactgatgcttttgatatctggggccaagggacaatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 112 - protein, 471 aa, Homo sapiens
MDMRVPAQLLGLLLLWLRGARCQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 702 bp, Homo sapiens
  source 1..702: mol_type = other DNA
atggaagcgccggcgcagcttctcttcctcctgctactctggctcccagataccactggagaaatagtgatgacgcagtctccagccaccctgtctgtgtctcctggggaaagagccaccctctcctgcagggccagtcagagtgttagcagcaacttagcctggttccagcagaaacctggccaggctcccaggcccctcatctatgatgcatccaccagggccactggtgtcccagccaggttcagtggcagtgggtctgggacagacttcactctcaccatcagcagcctgcagtctgaagattttgcagtttattactgtcagcagtatgataactggccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 114 - protein, 234 aa, Homo sapiens
MEAPAQLLFLLLLWLPDTTGEIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 1383 bp, Homo sapiens
  source 1..1383: mol_type = other DNA
atggagtggacctggagggtccttttcttggtggcagcagcaacaggtgcccactcccaggttcagctggtgcagtctggagctgaggtgaagaagcctggggcctcagtgaaggtctcctgcaaggcttctggttacacctttaccagatatggtatcagctgggtgcgacaggcccctggacaagggcttgagtggatgggatggatcagcacttacagtggtaacacaaactatgcacagaagctccagggcagagtcaccatgaccacagacacatccacgagcacagcctacatggagctgaggagcctgagatctgacgacacggccgtgtattactgtgcgagacggcagctttactttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 116 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - DNA, 717 bp, Homo sapiens
  source 1..717: mol_type = other DNA
atgaggctccctgctcagctcctggggctgctaatgctctggatacctggatccagtgcagatattgtgatgacccagactccactctctctgtccgtcacccctggacagccggcctccatctcctgcaagtctggtcagagcctcctgcatagtgatggaaagacctatttgtattggtacctgcagaagccaggccagcctccacagttcctgatctatgaagtttccaaccggttctctagagtgccagataggttcagtggcagcgggtcagggacagatttcacactgagaatcagccgggtggaggctgaggatgttggaatttattactgcatgcaaagtatacagcttccgtggacgttcggccaagggacccaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 118 - protein, 239 aa, Homo sapiens
MRLPAQLLGLLMLWIPGSSADIVMTQTPLSLSVTPGQPASISCKSGQSLLHSDGKTYLYWYLQKPGQPPQFLIYEVSNRFSRVPDRFSGSGSGTDFTLRISRVEAEDVGIYYCMQSIQLPWTFGQGTQVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - DNA, 1377 bp, Homo sapiens
  source 1..1377: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtggctatggcatgcactgggtccgccaggctccaggcaaggggctggagtgggtggcagttatatcatatgatggaaatgataaatactatgcagactccgtgaagggccgattcaccatctccagagacaatgccaagaacacgctgtatctgcaaatgaacagcctgagagctgaggacacggctgtgtattactgtgcgagagagctacgggtcctctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - protein, 459 aa, Homo sapiens
MEFGLSWVFLVALLRGVQCQVQLVESGGGVVQPGRSLRLSCAASGFTFSGYGMHWVRQAPGKGLEWVAVISYDGNDKYYADSVKGRFTISRDNAKNTLYLQMNSLRAEDTAVYYCARELRVLWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - DNA, 705 bp, Homo sapiens
  source 1..705: mol_type = other DNA
atggacatgagggtgcccgctcagctcctggggctcctgctgctgtggctgagaggtgccagatgtgacatccagatgacccagtctccatcctccctgtctgcatctgtaggtgaccgtgtcaccatcacttgccgcgcaagtcaggatattagcagctatttaaattggtatcagcagaaaccagggaaagcccctaagctcctgatctattctacttcccgtttgaatagtggggtcccatcacgcttcagtggcagtggctctgggacagatttcactctcaccatcagcagtctgcaacctgaagattttgcaacttactactgtcaacaggatattaaacaccctacgttcggtcaaggcaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 122 - protein, 235 aa, Homo sapiens
MDMRVPAQLLGLLLLWLRGARCDIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 1395 bp, Homo sapiens
  source 1..1395: mol_type = other DNA
atggactggacctggaggatcctcttcttggtggcagcagccacaggagcccactccgaggtgcagctggtgcagtctggggctgaggtgaagaagcctgggtcctcggtgaaggtctcctgcaaggcttctggttttaccttcaccgactatattatgcactgggtgcgtcaggcccctggtcaagggcttgagtggatgggctatatcaacccttataatgatgacaccgaatacaacgagaagttcaagggccgtgtcacgattaccgcggacaaatccacgagcacagcctacatggagctgagcagcctgcgctctgaggacacggccgtgtattactgtgcgcgttcgatttattactacgatgccccgtttgcttactggggccaagggactctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 124 - protein, 465 aa, Homo sapiens
MDWTWRILFLVAAATGAHSEVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 125 - DNA, 699 bp, Homo sapiens
  source 1..699: mol_type = other DNA
atggggtcaaccgccatccttggcctcctcctggctgtcctgcagggagggcgcgcctcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtattcggcggagggaccaagctgaccgtcctaggccaaccgaaagcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 126 - protein, 233 aa, Homo sapiens
MGSTAILGLLLAVLQGGRASYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 127 - DNA, 1401 bp, Homo sapiens
  source 1..1401: mol_type = other DNA
atggggtcaaccgccatccttggcctcctcctggctgtcctgcagggagggcgcgcccagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 128 - protein, 467 aa, Homo sapiens
MGSTAILGLLLAVLQGGRAQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 129 - DNA, 702 bp, Homo sapiens
  source 1..702: mol_type = other DNA
atgggtgtgcctactcatctcctgggtttgttgctgctctggattacacatgccatatgtgatatccggatgacacagtctccagcttccctgtctgcatctctgggagaaactgtcaacatcgaatgtctagcaagtgaggacatttacagtgatttagcatggtatcagcagaagccagggaaatctcctcagctcctgatctataatgcaaatagcttgcaaaatggggtcccttcacggtttagtggcagtggatctggcacacagtattctctaaaaataaacagcctgcaatctgaagatgtcgcgacttatttctgtcaacaatataacaattatcctccgacgttcggtggaggcaccaagctggaattgaaacgggctgatgctgcaccaactgtatctatcttcccaccatccacggaacagttagcaactggaggtgcctcagtcgtgtgcctcatgaacaacttctatcccagagacatcagtgtcaagtggaagattgatggcactgaacgacgagatggtgtcctggacagtgttactgatcaggacagcaaagacagcacgtacagcatgagcagcaccctctcgttgaccaaggctgactatgaaagtcataacctctatacctgtgaggttgttcataagacatcatcctcacccgtcgtcaagagcttcaacaggaatgagtgt

SEQ ID NO: 130 - protein, 234 aa, Homo sapiens
MGVPTHLLGLLLLWITHAICDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 131 - DNA, 1395 bp, Homo sapiens
  source 1..1395: mol_type = other DNA
atggacatcaggctcagcttggctttccttgtccttttcataaaaggtgtccagtgtgaggtacagctggtggagtctggcggaggattggtacagcctgcaaactccctgaaactctcctgtgcagcctcaggattcactttcagtgactatgccatggcctgggtccgccagtctccaaagaagggtctggagtgggtcgcaaccattatttatgatggtagtagcacttactatcgagactccgtgaagggccgattcactatctccagagataatgcaaaaagcaccctatacctgcaaatggacagtctgaggtctgaggacacggccacttattactgtgcaacaggtctgggtatagctacggactactttgattactggggccaaggagtcctggtcacagtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctgggctctacactctcaccagctcagtgactgtaccctccagcacctggcccagccagaccgtcacctgcaacgtagcccacccggccagcagcaccaaggtggacaagaaaattgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgacccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaa

SEQ ID NO: 132 - protein, 465 aa, Homo sapiens
MDIRLSLAFLVLFIKGVQCEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 133 - DNA, 711 bp, Homo sapiens
  source 1..711: mol_type = other DNA
atggacatgagggtgcccgctcagctcctggggctcctgctgctgtggctgagaggtgccagatgtgagtctgccctgactcagcctgcctccgtgtctgggtctcctggacagtcgatcaccatctcctgcactggaaccagcagtgacgttggtggttataactctgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcaattcatatacaagcaccagcatggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 134 - protein, 237 aa, Homo sapiens
MDMRVPAQLLGLLLLWLRGARCESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 135 - DNA, 1380 bp, Homo sapiens
  source 1..1380: mol_type = other DNA
atggactggacctggaggatccttttcttggtggcagcagccacaggtgtccactccgaggttcagctggtgcagtctggagctgaggtgaagaagcctggggcctcagtgaaggtctcctgcaaggcttctggttacaccttaaccagctatggtatcagctgggtgcgacaggcccctggacaagggcttgagtggatgggatgggtcagtttttataatggtaacacaaactatgcacagaagctccagggcagaggcaccatgaccacagacccatccacgagcacagcctacatggagctgaggagcctgagatctgacgacacggccgtgtattactgtgcgagaggctacggtatggacgtctggggccaagggaccacggtcaccgtctcctctgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 136 - protein, 460 aa, Homo sapiens
MDWTWRILFLVAAATGVHSEVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 137 - DNA, 693 bp, Homo sapiens
  source 1..693: mol_type = other DNA
atggcatggatccctctcttcctcggcgtccttgcttactgcacaggatccgtggcctcctatgaggtgactcaggcaccctcagtgtccgtgtccccaggacagacagccagcatcacctgctctggagataaattgggggataaatatgcttgttggtatcagcagaagccaggccagtcccctgtgctggtcatctatcaagatagcaagcggccctcagggatccctgagcgattctctggctccaactctggaaacacagccactctgaccatcagcgggacccaggctatggatgaggctgactattactgtcaggcgtgggacagcagcactgcggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 138 - protein, 231 aa, Homo sapiens
MAWIPLFLGVLAYCTGSVASYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 139 - DNA, 1401 bp, Homo sapiens
  source 1..1401: mol_type = other DNA
atggactggacctggagcatccttttcttggtggcagcagcaacaggtgcccactcccaggttcagctggtgcagtctggagctgaggtgaagaagcctggggcctcagtgaaggtctcctgcaaggcttctggttacacctttaccagttatggtctcagctgggtgcgacaggcccctggacaagggcttgagtggatgggatggatcatcccttacaatggtaacacaaactctgcacagaaactccagggcagagtcaccatgaccacagacacatccacgagcacagcctacatggagctgaggagcctgagatctgacgacacggccgtgtatttctgtgcgagagacagggactacggtgtcaattatgatgcttttgatatctggggccaagggacaatggtcaccgtctcttcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 140 - protein, 467 aa, Homo sapiens
MDWTWSILFLVAAATGAHSQVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 141 - DNA, 705 bp, Homo sapiens
  source 1..705: mol_type = other DNA
atggcctgggctccactacttctcaccctcctcgctcactgcacaggttcttgggccaattttatgctgactcagccccactctgtgtcggagtctccggggaagacggtggccatctcctgcacccgcaacagtggcagcattgccagcaactctgtgcagtggtaccagcagcgcccgggcagttcccccaccactgtgatctttgaggataaccaaagaccctctggggtccctgatcggttctctggctccatcgacagctcctccaactctgcctccctcaccatctctggtctgaagactgaggacgaggctgactactactgtcagtcttatgatagcaacaattgggtgttcggcggagggaccaaactgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 142 - protein, 235 aa, Homo sapiens
MAWAPLLLTLLAHCTGSWANFMLTQPHSVSESPGKTVAISCTRNSGSIASNSVQWYQQRPGSSPTTVIFEDNQRPSGVPDRFSGSIDSSSNSASLTISGLKTEDEADYYCQSYDSNNWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 143 - DNA, 1410 bp, Homo sapiens
  source 1..1410: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatgtcatgcactgggtccgccaggctccaggcaaggggctggagtgggtggctgttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagaggggtatgactacggtgaagactactactactacggtatggacgtctggggccaagggaccacggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 144 - protein, 470 aa, Homo sapiens
MEFGLSWVFLVALLRGVQCQVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCAREGYDYGEDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 145 - DNA, 708 bp, Homo sapiens
  source 1..708: mol_type = other DNA
atggacatgagggtccccgctcagctcctggggctcctgctactctggctccgaggtgccagatgtgacatccagatgacccagtctccatcctccctctccgcatccgtaggcgaccgcgtaaccataacatgtagagcatctcaagatatttccaactatttgaattggtaccaacaaaaacccggcaaagcacctaaactcctcatttactatacatcaagactcctctccggcgttccatcacgattctcaggctccggctccggcacagatttcacactcactatttcctccctccaaccagaagattttgcaacctattactgtcaacaaggcgatacactcccatacacattcggcggcggcacaaaagttgaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 146 - protein, 236 aa, Homo sapiens
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - DNA, 1404 bp, Homo sapiens
  source 1..1404: mol_type = other DNA
atggactggacctggaggatcctcttcttggtggcagcagccacaggagcccactccgaggtgcagctggtgcagagcggcgccgaggtaaaaaaaccaggagcaagcgttaaagtttcttgtaaagcaagcggatatacatttacagattacaacatgcattgggtaagacaagcgccaggacaaggattggaatggatgggcgaaattaaccctaatagtggaggagcaggctacaatcaaaaattcaaagggagagttacaatgacaacagacacaagcacttcaacagcatatatggaactgcgatcacttagaagcgacgatacagctgtatactattgcgcacgacttgggtatgatgatatatatgatgactggtatttcgatgtttggggccagggaacaacagttaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 148 - protein, 468 aa, Homo sapiens
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 149 - DNA, 720 bp, Homo sapiens
  source 1..720: mol_type = other DNA
atggtgttgcagacccaggtcttcatttctctgttgctctggatctctggtgcctacggggacatcgtgatgacccagtctccagactccctggctgtgtctctgggcgagagggccaccatcaactgcaagtccagccagagtgttttagacagctccgacaataagaactacttagcttggtaccagcagaaaccaggacagcctcctaagctgctcatttactgggcatctaaccgggaatccggggtccctgaccgattcagtggcagcgggtctgggacagatttcactctcaccatcagcagcctgcaggctgaagatgtggcagtttattactgtcagcaatattatagtgatccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 150 - protein, 240 aa, Homo sapiens
MVLQTQVFISLLLWISGAYGDIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 1395 bp, Homo sapiens
  source 1..1395: mol_type = other DNA
atggactggacctggagcatccttttcttggtggcagcaccaacaggtgcccactcccaggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagagtcgtggttcggggaggtattctttgactactggggccagggaaccctggtcaccgtctcctcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - protein, 465 aa, Homo sapiens
MDWTWSILFLVAAPTGAHSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 153 - DNA, 708 bp, Homo sapiens
  source 1..708: mol_type = other DNA
atggacatgagggtgcccgctcagctcctggggctcctgctgctgtggctgagaggtgcgcgctgtgacatccagatgacccagtctccatcttccgtgtctgcatctgtaggagacagagtcaccatcacttgtcgggcgagtcagggtattagcagctggttagcctggtatcaacagaaaccagggaaagcccctaagctcctgatctatggtgcatccaatttggaaagtggggtcccatcaaggttcagcggcagtggatctgggacagatttcactctcaccatcagcagcctgcagcctgaagattttgcaaattactattgtcaacaggctaacagtttcccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 154 - protein, 236 aa, Homo sapiens
MDMRVPAQLLGLLLLWLRGARCDIQMTQSPSSVSASVGDRVTITCRASQGISSWLAWYQQKPGKAPKLLIYGASNLESGVPSRFSGSGSGTDFTLTISSLQPEDFANYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - DNA, 1398 bp, Homo sapiens
  source 1..1398: mol_type = other DNA
atggacatgagggtgcccgctcagctcctggggctcctgctgctgtggctgagaggtgcgcgctgtcaggtccagctggtacagtctggggctgaggtgaagaagcctggggcctcagtgaaggtctcctgcaaggtttccggatacaccctcagtgatttatccatccactgggtgcgacaggctcctggaaaagggcttgagtggatgggaggttttgatcctcaagatggtgaaacaatctacgcacagaagttccagggcagagtcaccatgaccgaggacacatctacagacacagcctacatggagctgagcagcctgaaatctgaggacacggccgtgtattactgcgcaacggggagcagctcgtcctggttcgacccctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 156 - protein, 466 aa, Homo sapiens
MDMRVPAQLLGLLLLWLRGARCQVQLVQSGAEVKKPGASVKVSCKVSGYTLSDLSIHWVRQAPGKGLEWMGGFDPQDGETIYAQKFQGRVTMTEDTSTDTAYMELSSLKSEDTAVYYCATGSSSSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 157 - DNA, 702 bp, Homo sapiens
  source 1..702: mol_type = other DNA
atggagacccctgcccagctgctgttcctgctgctgctgtggctgcccgacacaaccggcgaaatcgtcatgacacagagccctgccacactgtccgtgagccctggagagagggctaccctgagctgcagggcttcccagagcgtgagcagcaacctggcctggtaccaacagaagcctggccaggcccctaggctgctgatctacggcgctgctaccagggccaccggtattcctgccagggtgtccggctccggatccggcaccgagtttaccctgaccatcagcagcctgcagagcgaggacttcgccgtgtactactgtcagcaatacaacaactggcccctgacctttggcggcggcaccaaggtggagatcaagaggacagtggccgcccccagcgtgttcatcttccctcccagcgacgagcagctgaagagcggcaccgccagcgtggtgtgtctgctgaacaacttctaccccagggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccctgtgaccaagagcttcaacaggggcgagtgc

SEQ ID NO: 158 - protein, 234 aa, Homo sapiens
METPAQLLFLLLLWLPDTTGEIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - DNA, 1416 bp, Homo sapiens
  source 1..1416: mol_type = other DNA
atggatatgcgggtccctgctcagctgctgggactgctgctgctgtggctgaggggcgccaggtgtcaggtgcagctggtcgaaagcggaggaggagtggtgcagcccggaaggtccctgaggctgtcctgcgccgctagcggcttcaccttttccaactacggcatgcactgggtgaggcaagcccctggagagggcctggaatgggtggctgctatctggttcgacgccagcgacaagtactatgccgacgctgtgaagggccggttcaccatcagcagggacaacagcaagaacaccctctacctgcagatgaacagcctccgggccgaggacaccgctgtctattactgtgccagggaccaggccatcttcggagtggtccccgattactggggccagggaaccctggtgaccgtgtcctccgcttccacaaagggacctagcgtgttccctctggcccctagcagcaagtccacaagcggaggaacagccgccctgggctgtctcgtgaaggactattttcccgagcccgtgaccgtgtcctggaactccggagccctgacctccggcgtgcatacattccccgctgtcctgcagtccagcggcctctactccctgtcctccgtggtcaccgtgcctagcagcagcctgggcacccagacatacatctgcaacgtcaaccacaagccttccaacaccaaggtggacaagaaggtggagcccaagtcctgtgacaagacccacacctgtcctccctgtcctgctcctgagctgctgggaggcccctccgtcttcctgttccctcccaagcccaaggacaccctgatgatctccaggacccctgaagtgacatgtgtggtggtggatgtgagccacgaagatcccgaggtgaagttcaactggtacgtggacggcgtggaggtgcacaacgctaaaacaaagccctgcgaggagcagtacggatccacctacaggtgcgtgtccgtgctcaccgtgctccatcaggactggctgaacggaaaagagtacaagtgcaaagtcagcaataaggccctgcccgcccctatcgagaaaaccatcagcaaggccaaaggccagcccagggagcctcaggtgtataccctgcctccctccagggaggagatgaccaagaaccaggtgagcctgacctgcctcgtgaagggcttttatccctccgacatcgctgtggagtgggagagcaatggccagcctgaaaacaactacaaaaccaccccccctgtgctggatagcgacggcagcttcttcctctactccaagctgaccgtcgataagtcccggtggcagcagggcaacgtgtttagctgcagcgtgatgcacgaagccctgcataaccactacacccagaagagcctgagcctcagccccggaaag

SEQ ID NO: 160 - protein, 472 aa, Homo sapiens
MDMRVPAQLLGLLLLWLRGARCQVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 702 bp, Homo sapiens
  source 1..702: mol_type = other DNA
atggagacccctgcccagctgctgttcctgctgctgctgtggctgcccgacacaaccggcgaaatcgtcctgacacagagccctgccacactgtccctgagccctggagagagggctaccctgagctgcagggcttcccagagcgtgagcagcaacctggcctggtaccaacagaagcctggccaggcccctaggctgctgatctacggcgctgctaccagggccaccggtattcctgacagggtgtccggctccggatccggcaccgagtttaccctgaccatcagccgcctggagcccgaggacttcgccgtgtactactgtcagcaatacaacaactggcccctgacctttggcggcggcaccaaggtggagatcaagaggacagtggccgcccccagcgtgttcatcttccctcccagcgacgagcagctgaagagcggcaccgccagcgtggtgtgtctgctgaacaacttctaccccagggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccctgtgaccaagagcttcaacaggggcgagtgc

SEQ ID NO: 162 - protein, 234 aa, Homo sapiens
METPAQLLFLLLLWLPDTTGEIVLTQSPATLSLSPGERATLSCRASQSVSSNLAWYQQKPGQAPRLLIYGAATRATGIPDRVSGSGSGTEFTLTISRLEPEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - DNA, 1416 bp, Homo sapiens
  source 1..1416: mol_type = other DNA
atggatatgcgggtccctgctcagctgctgggactgctgctgctgtggctgaggggcgccaggtgtgaggtgcagctggtcgaaagcggaggaggagtggtgcagcccggagggtccctgaggctgtcctgcgccgctagcggcttcaccttttccaactacggcatgcactgggtgaggcaagcccctggagagggcctggaatgggtggctgctatctggttcgacgccagcgacaagtactatgccgacgctgtgaagggccggttcaccatcagcagggacaacgccaagaacaccctctacctgcagatgaacagcctccgggccgaggacaccgctgtctattactgtgccagggaccaggccatcttcggagtggtccccgattactggggccagggaaccctggtgaccgtgtcctccgcttccacaaagggacctagcgtgttccctctggcccctagcagcaagtccacaagcggaggaacagccgccctgggctgtctcgtgaaggactattttcccgagcccgtgaccgtgtcctggaactccggagccctgacctccggcgtgcatacattccccgctgtcctgcagtccagcggcctctactccctgtcctccgtggtcaccgtgcctagcagcagcctgggcacccagacatacatctgcaacgtcaaccacaagccttccaacaccaaggtggacaagaaggtggagcccaagtcctgtgacaagacccacacctgtcctccctgtcctgctcctgagctgctgggaggcccctccgtcttcctgttccctcccaagcccaaggacaccctgatgatctccaggacccctgaagtgacatgtgtggtggtggatgtgagccacgaagatcccgaggtgaagttcaactggtacgtggacggcgtggaggtgcacaacgctaaaacaaagccctgcgaggagcagtacggatccacctacaggtgcgtgtccgtgctcaccgtgctccatcaggactggctgaacggaaaagagtacaagtgcaaagtcagcaataaggccctgcccgcccctatcgagaaaaccatcagcaaggccaaaggccagcccagggagcctcaggtgtataccctgcctccctccagggaggagatgaccaagaaccaggtgagcctgacctgcctcgtgaagggcttttatccctccgacatcgctgtggagtgggagagcaatggccagcctgaaaacaactacaaaaccaccccccctgtgctggatagcgacggcagcttcttcctctactccaagctgaccgtcgataagtcccggtggcagcagggcaacgtgtttagctgcagcgtgatgcacgaagccctgcataaccactacacccagaagagcctgagcctcagccccggaaag

SEQ ID NO: 164 - protein, 472 aa, Homo sapiens
MDMRVPAQLLGLLLLWLRGARCEVQLVESGGGVVQPGGSLRLSCAASGFTFSNYGMHWVRQAPGEGLEWVAAIWFDASDKYYADAVKGRFTISRDNAKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - DNA, 717 bp, Homo sapiens
  source 1..717: mol_type = other DNA
atgaggctccctgctcagctcctggggctgctaatgctctgggtccctggatccagtggggatattgtgatgacccagactccactctcctcacctgtcacccttggacagccggcctccatctcctgcaggtctagtcaaagcctcgtacacagtgatggaaacacctacttgaattggcttcagcagaggccaggccagcctccaagactcctaatttataagatttctaaccggttctctggggtcccagacagattcactggcagtggggcagggacagatttcacactgaaaatcagcagggtggaagctgaggatgtcggggtttatacctgcatgcaagttacacaatttcctctcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 166 - protein, 239 aa, Homo sapiens
MRLPAQLLGLLMLWVPGSSGDIVMTQTPLSSPVTLGQPASISCRSSQSLVHSDGNTYLNWLQQRPGQPPRLLIYKISNRFSGVPDRFTGSGAGTDFTLKISRVEAEDVGVYTCMQVTQFPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - DNA, 1410 bp, Homo sapiens
  source 1..1410: mol_type = other DNA
atggacacactttgctacacactcctgctgctgaccaccccttcctgggtcttgtcccaggtcaccttgaaggagtctggtcctgtgctgctgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacatcacatctctgaagagcaggctcaccatctccaaggacacctccaaaagccaggtggtccttaccatgaccaacatggaccctgtggacacagccacatattactgtgcacggatacccctacgatccccgggtgcttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctcactgtctccgggtaaa

SEQ ID NO: 168 - protein, 470 aa, Homo sapiens [sequence omitted]

SEQ ID NO: 169 - DNA, 705 bp, Homo sapiens
  source 1..705: mol_type = other DNA
atggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 170 - protein, 235 aa, Homo sapiens
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - DNA, 1401 bp, Homo sapiens
  source 1..1401: mol_type = other DNA
atggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 172 - protein, 467 aa, Homo sapiens
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 173 - DNA, 714 bp, Homo sapiens
  source 1..714: mol_type = other DNA
atggtgttgcagacccaggtcttcatttctctgttgctctggatctctggtgcctacggggacatcgtgatgacccagtctccagactccctggctgtgtctctgggcgagagggccaccatcaactgcagagccagtgaaagtgttgatatttatggcaatagttttatgcactggtaccagcagaaaccaggacagcctcctaagctgctcatttaccttgcatccaacctagaatctggggtccctgaccgattcagtggcagcgggtctgggacagatttcactctcaccatcagcagcctgcaggctgaagatgtggcagtttattactgtcagcaaaataatgaggatccgtacacgttcggaggtgggaccaaggtggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 174 - protein, 238 aa, Homo sapiens
MVLQTQVFISLLLWISGAYGDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - DNA, 1398 bp, Homo sapiens
  source 1..1398: mol_type = other DNA
atggactggacctggagggtcttctgcttgctggcagtggccccaggtgcccactcccaggtgcagctggtgcagtctggggctgaggtgaagaagcctggggcctcagtgaaggtctcctgcaaggcttctggatacaccttcaccagttacaatatgcactgggtgcgccaggcccctggacaagggcttgagtggatgggagttatttattcaggaaatggtgatacttcctacaatcagaagttcaaaggcagggtcaccattaccgctgacaaatccaccagcacagcctacatggagctgagcagcctgagatctgaggacacggccgtgtattactgtgcgagagagagggatactcgttttggtaactggggccaagggactctggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 176 - protein, 466 aa, Homo sapiens
MDWTWRVFCLLAVAPGAHSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - DNA, 705 bp, Homo sapiens
  source 1..705: mol_type = other DNA
atggcctgggctctgctgctcctcactctcctcactcaggacacagggtcctgggcccagtctgtgctgactcagtcaccctcagcgtctgggacccccgggcagagagtcaccatctcttgttctggaagcagctccaacatcggcagtaattatgtatactggtaccagcagctcccaggagcggcccccaaactcctcatccttaggaataatcagcggccctcaggggtccctgaccgattctctggctccaagtctggcacctcagcctccctgaccatcagtgggctccggtccgaggatgaggctgactattattgtgcagcatgggatgacagcctgagtggttgggtgttcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 178 - protein, 235 aa, Homo sapiens
MAWALLLLTLLTQDTGSWAQSVLTQSPSASGTPGQRVTISCSGSSSNIGSNYVYWYQQLPGAAPKLLILRNNQRPSGVPDRFSGSKSGTSASLTISGLRSEDEADYYCAAWDDSLSGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 179 - DNA, 1437 bp, Homo sapiens
  source 1..1437: mol_type = other DNA
atggacatgagggtgcccgctcagctcctggggctcctgctgctgtggctgagaggtgcgcgctgtgaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacactgcacccgtgaaaggcagattcaccatctcaagagatgattcaaaaaacacgctgtatctgcaaatgaatagcctgaaagccgaggacacagccgtgtattactgtaccacagatcggaccgggtatagcatcagctggtctagttactactactactacggtatggacgtctggggccaagggaccacggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - protein, 479 aa, Homo sapiens
MDMRVPAQLLGLLLLWLRGARCEVQLVESGGGLVKPGGSLRLSCAASGFTFSNAWMSWVRQAPGKGLEWVGRIKSKTDGGTTDYTAPVKGRFTISRDDSKNTLYLQMNSLKAEDTAVYYCTTDRTGYSISWSSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 181 - protein, 23 aa, Homo sapiens
EIVMTQSPATLSVSPGERATLSC

SEQ ID NO: 182 - protein, 15 aa, Homo sapiens
WYRQKPGQAPRLLIY

SEQ ID NO: 183 - protein, 32 aa, Homo sapiens
GIPARFSGSGSGTEFTLTISSLQSEDFAVYYC

SEQ ID NO: 184 - protein, 10 aa, Homo sapiens
FGQGTRLEIK

SEQ ID NO: 185 - protein, 23 aa, Homo sapiens
EIVLTQSPDFQSVTPKEKVTITC

SEQ ID NO: 186 - protein, 15 aa, Homo sapiens
WYQQKPDQSPKLLIK

SEQ ID NO: 187 - protein, 32 aa, Homo sapiens
GVPSRFSGSGSGTDFTLTINSLEAEDAAAYYC

SEQ ID NO: 188 - protein, 10 aa, Homo sapiens
FGGGTKVEIK

SEQ ID NO: 189 - protein, 23 aa, Homo sapiens
DIVMTQTPLSSPVTLGQPASISC

SEQ ID NO: 190 - protein, 15 aa, Homo sapiens
WLQQRPGQPPRLLIY

SEQ ID NO: 191 - protein, 32 aa, Homo sapiens
GVPDRFTGSGAGTDFTLKISRVEAEDVGVYTC

SEQ ID NO: 192 - protein, 10 aa, Homo sapiens
FGQGTRLEIK

SEQ ID NO: 193 - protein, 23 aa, Homo sapiens
EIVLTQSPGTLSLSPGERATLSC

SEQ ID NO: 194 - protein, 15 aa, Homo sapiens
WYQQKPGQAPRLLIY

SEQ ID NO: 195 - protein, 32 aa, Homo sapiens
GIPDRFSGSGSGTDFTLTISRLEPEDFAVFYC

SEQ ID NO: 196 - protein, 10 aa, Homo sapiens
FGQGTKVEIK

SEQ ID NO: 197 - protein, 23 aa, Homo sapiens
EIVLTQSPGTLSLSPGDRATLSC

SEQ ID NO: 198 - protein, 32 aa, Homo sapiens
GIPDRFSGSGSGTDFTLTISRLEPEDFAVYYC

SEQ ID NO: 199 - protein, 15 aa, Homo sapiens
WHQQKPGQAPRLLIY

SEQ ID NO: 200 - protein, 15 aa, Homo sapiens
WHQQKPGQAPRLLIY

SEQ ID NO: 201 - protein, 32 aa, Homo sapiens
GIPDRFSGSGSGTDFTLTVSRLEPEDFAVYYC

SEQ ID NO: 202 - protein, 15 aa, Homo sapiens
WFQQKPGQAPRLLIY

SEQ ID NO: 203 - protein, 32 aa, Homo sapiens
GIPDRFSGSGSGTDFTFTISRLEPEDFAVYYC

SEQ ID NO: 204 - protein, 10 aa, Homo sapiens
FGQGTTVEIK

SEQ ID NO: 205 - protein, 23 aa, Homo sapiens
DIQMTQSPSSLSASVGDRVTITC

SEQ ID NO: 206 - protein, 15 aa, Homo sapiens
WYQQKPGKAPKLLIY

SEQ ID NO: 207 - protein, 32 aa, Homo sapiens
GVPSRFSGSGSGTDFTLTISSLQPEDFATYYC

SEQ ID NO: 208 - protein, 23 aa, Homo sapiens
DIQMTQSPSSVSASVGDRVTITC

SEQ ID NO: 209 - protein, 32 aa, Homo sapiens
GVPSRFSGSGSGTDFTLTISSLQPEDFANYYC

SEQ ID NO: 210 - protein, 23 aa, Homo sapiens
DIVMTQSPDSLAVSLGERATINC

SEQ ID NO: 211 - protein, 15 aa, Homo sapiens
WYQQKPGQPPKLLIY

SEQ ID NO: 212 - protein, 32 aa, Homo sapiens
GVPDRFSGSGSGTDFTLTISSLQAEDVAVYYC

SEQ ID NO: 213 - protein, 32 aa, Homo sapiens
GVPDRFSGSGAGTDFTLKISRVEAEDVGVYYC

SEQ ID NO: 214 - protein, 10 aa, Homo sapiens
FGPGTKVDIK

SEQ ID NO: 215 - protein, 23 aa, Homo sapiens
DIVLTQSPDSLAVSLGERATINC

SEQ ID NO: 216 - protein, 32 aa, Homo sapiens
GVPDRFSGSGSGTDFTLTISSLQPEDVAVYYC

SEQ ID NO: 217 - protein, 10 aa, Homo sapiens
FGQGTRVEIK

SEQ ID NO: 218 - protein, 23 aa, Homo sapiens
DIQLTQSPSSLSASVGDRVTMSC

SEQ ID NO: 219 - protein, 32 aa, Homo sapiens
GVPSRFSGSGSGTDFTFTISSLQPEDIATYYC

SEQ ID NO: 220 - protein, 10 aa, Homo sapiens
FGGGTKVQIK

SEQ ID NO: 221 - protein, 32 aa, Homo sapiens
GVPSRFSGSGSGTDFTFTISSLQPEDIATYFC

SEQ ID NO: 222 - protein, 15 aa, Homo sapiens
WYQQKPGKAPKRLIY

SEQ ID NO: 223 - protein, 32 aa, Homo sapiens
GVPSRFSGSGSGTEFTLTISSVQPEDFVTYYC

SEQ ID NO: 224 - protein, 23 aa, Homo sapiens
DVVMTQSPLSLPVTPGEPASISC

SEQ ID NO: 225 - protein, 15 aa, Homo sapiens
WYLQKPGQSPQLLIY

SEQ ID NO: 226 - protein, 32 aa, Homo sapiens
GVPDRFSGSGSGTDFTLKISRVEAEDVGVYYC

SEQ ID NO: 227 - protein, 23 aa, Homo sapiens
DIVMTQSPLSLPVTPGEPASISC

SEQ ID NO: 228 - protein, 32 aa, Homo sapiens
GVPDRFSGSGSGTHLTLKISRVEAEDVGVYYC

SEQ ID NO: 229 - protein, 15 aa, Homo sapiens
WYQQKPEKAPKSLIY

SEQ ID NO: 230 - protein, 10 aa, Homo sapiens
FGQGTKLEIS

SEQ ID NO: 231 - protein, 10 aa, Homo sapiens
FGQGTKLEIK

SEQ ID NO: 232 - protein, 15 aa, Homo sapiens
WFQQKPGQAPRPLIY

SEQ ID NO: 233 - protein, 32 aa, Homo sapiens
GVPARFSGSGSGTDFTLTISSLQSEDFAVYYC

SEQ ID NO: 234 - protein, 32 aa, Homo sapiens
GIPARVSGSGSGTEFTLTISSLQSEDFAVYYC

SEQ ID NO: 235 - protein, 23 aa, Homo sapiens
DVLMTQSPLSLPVTLGQPASISC

SEQ ID NO: 236 - protein, 15 aa, Homo sapiens
WYLQRPGQSPKLLIY

SEQ ID NO: 237 - protein, 10 aa, Homo sapiens
FGAGTKLEIK

SEQ ID NO: 238 - protein, 23 aa, Homo sapiens
EIVLTQSPATLSLSPGERATLSC

SEQ ID NO: 239 - protein, 32 aa, Homo sapiens
GIPARFSGSGSGTDFTLTISSLEPEDFAVYYC

SEQ ID NO: 240 - protein, 23 aa, Homo sapiens
DIVMTQTPLSLSVTPGQPASISC

SEQ ID NO: 241 - protein, 15 aa, Homo sapiens
WYLQKPGQPPQFLIY

SEQ ID NO: 242 - protein, 32 aa, Homo sapiens
RVPDRFSGSGSGTDFTLRISRVEAEDVGIYYC

SEQ ID NO: 243 - protein, 10 aa, Homo sapiens
FGQGTQVEIK

SEQ ID NO: 244 - protein, 32 aa, Homo sapiens
GIPDRVSGSGSGTEFTLTISRLEPEDFAVYYC

SEQ ID NO: 245 - protein, 15 aa, Homo sapiens
WYQQKPGTAPKLLIY

SEQ ID NO: 246 - protein, 23 aa, Homo sapiens
DIVMTQTPLSLPVTPGEPASISC

SEQ ID NO: 247 - protein, 22 aa, Homo sapiens
ESALTQPASVSGSPGQSITISC

SEQ ID NO: 248 - protein, 15 aa, Homo sapiens
WYQQHPGKAPKLMIY

SEQ ID NO: 249 - protein, 32 aa, Homo sapiens
GVSNRFSGSKSGNTASLTISGLQAEDEADYYC

SEQ ID NO: 250 - protein, 10 aa, Homo sapiens
FGGGTKLTVL

SEQ ID NO: 251 - protein, 22 aa, Homo sapiens
SYVLTQPPSVSVAPGQTARITC

SEQ ID NO: 252 - protein, 15 aa, Homo sapiens
WYQQKPGQAPVLVVY

SEQ ID NO: 253 - protein, 32 aa, Homo sapiens
WIPERFSGSNSGNTATLTISRGEAGDEADYYC

SEQ ID NO: 254 - protein, 22 aa, Homo sapiens
QSVLTQPPSVSAAPGQKVTISC

SEQ ID NO: 255 - protein, 15 aa, Homo sapiens
WYQQLPGTAPKLLIY

SEQ ID NO: 256 - protein, 32 aa, Homo sapiens
GIPDRFSGSKSGTSTTLGITGLQTGDEADYYC

SEQ ID NO: 257 - protein, 22 aa, Homo sapiens
QSVLTQSPSASGTPGQRVTISC

SEQ ID NO: 258 - protein, 15 aa, Homo sapiens
WYQQLPGAAPKLLIL

SEQ ID NO: 259 - protein, 32 aa, Homo sapiens
GVPDRFSGSKSGTSASLTISGLRSEDEADYYC

SEQ ID NO: 260 - protein, 22 aa, Homo sapiens
SYEVTQAPSVSVSPGQTASITC

SEQ ID NO: 261 - protein, 15 aa, Homo sapiens
WYQQKPGQSPVLVIY

SEQ ID NO: 262 - protein, 32 aa, Homo sapiens
GIPERFSGSNSGNTATLTISGTQAMDEADYYC

SEQ ID NO: 263 - protein, 22 aa, Homo sapiens
CYELTQPPSVSVSPGQTATITC

SEQ ID NO: 264 - protein, 15 aa, Homo sapiens
WYQQRPGQSPVLVIY

SEQ ID NO: 265 - protein, 32 aa, Homo sapiens
GIPERFSGSNSGNTATLTISGTQAMDEADYFC

SEQ ID NO: 266 - protein, 22 aa, Homo sapiens
SSELTQDPTVSVALGQTVKITC

SEQ ID NO: 267 - protein, 15 aa, Homo sapiens
WYQQKPGQAPVLVFY

SEQ ID NO: 268 - protein, 32 aa, Homo sapiens
GIPDRFSGSSSGNTASLTITGAQAEDEADYYC

SEQ ID NO: 269 - protein, 10 aa, Homo sapiens
LGGGTKLTVL

SEQ ID NO: 270 - protein, 22 aa, Homo sapiens
NFMLTQPHSVSESPGKTVAISC

SEQ ID NO: 271 - protein, 15 aa, Homo sapiens
WYQQRPGSSPTTVIF

SEQ ID NO: 272 - protein, 34 aa, Homo sapiens
GVPDRFSGSIDSSSNSASLTISGLKTEDEADYYC

SEQ ID NO: 273 - protein, 30 aa, Homo sapiens
QVQLQESGPGLVKPSETLSLTCTVSGGSIS

SEQ ID NO: 274 - protein, 14 aa, Homo sapiens
WIRQPPGKGLEWIG

SEQ ID NO: 275 - protein, 32 aa, Homo sapiens
RVTISVDTSKNQFSLKLNSVTAADTAVYYCAR

SEQ ID NO: 276 - protein, 11 aa, Homo sapiens
WGQGTLVTVSS

SEQ ID NO: 277 - protein, 30 aa, Homo sapiens
EVQLMQSGAEVKKPGESLKISCKGSGYSFS

SEQ ID NO: 278 - protein, 14 aa, Homo sapiens
WVRQMPGKGLEWMG

SEQ ID NO: 279 - protein, 32 aa, Homo sapiens
QVTISADNSNSATYLQWSSLKASDTAMYFCAR

SEQ ID NO: 280 - protein, 30 aa, Homo sapiens
QVTLKESGPVLLKPTETLTLTCTVSGFSLS

SEQ ID NO: 281 - protein, 14 aa, Homo sapiens
WIRQPPGKALEWLA

SEQ ID NO: 282 - protein, 32 aa, Homo sapiens
RLTISKDTSKSQVVLTMTNMDPVDTATYYCAR

SEQ ID NO: 283 - protein, 11 aa, Homo sapiens
WGQGTMVTVSS

SEQ ID NO: 284 - protein, 30 aa, Homo sapiens
EVQLVQSGAEVKKPGASVKVSCKASGYTLT

SEQ ID NO: 285 - protein, 14 aa, Homo sapiens
WVRQAPGQGLEWMG

SEQ ID NO: 286 - protein, 32 aa, Homo sapiens
RGTMTTDPSTSTAYMELRSLRSDDTAVYYCAR

SEQ ID NO: 287 - protein, 11 aa, Homo sapiens
WGQGTTVTVSS

SEQ ID NO: 288 - protein, 30 aa, Homo sapiens
QMQLVESGGGVVQPGRSLRLSCAASGFTFR

SEQ ID NO: 289 - protein, 14 aa, Homo sapiens
WVRQAPGKGLEWVA

SEQ ID NO: 290 - protein, 32 aa, Homo sapiens
RFTITRDNSKNTLNLQMNSLRAEDTAVYYCAR

SEQ ID NO: 291 - protein, 30 aa, Homo sapiens
EVQLLESGGGLVQPGGSLRLSCAASGFTFS

SEQ ID NO: 292 - protein, 14 aa, Homo sapiens
WVRQAPGKGLEWVS

SEQ ID NO: 293 - protein, 32 aa, Homo sapiens
RFTISRDNSKNTLYLQMNSLRAEDTAVYYCAK

SEQ ID NO: 294 - protein, 30 aa, Homo sapiens
EVQLVQSGAEVKKPGSSVKVSCKASGFTFT

SEQ ID NO: 295 - protein, 32 aa, Homo sapiens
RVTITADKSTSTAYMELSSLRSEDTAVYYCAR

SEQ ID NO: 296 - protein, 30 aa, Homo sapiens
QVQLVQSGAEVKKPGASVKVSCKVSGYTLS

SEQ ID NO: 297 - protein, 14 aa, Homo sapiens
WVRQAPGKGLEWMG

SEQ ID NO: 298 - protein, 32 aa, Homo sapiens
RVTMTEDTSTDTAYMELSSLKSEDTAVYYCAT

SEQ ID NO: 299 - protein, 30 aa, Homo sapiens
QVQLVQSGAEVKKPGASVKVSCKASGYTFT

SEQ ID NO: 300 - protein, 30 aa, Homo sapiens
QVQLVQSGAEVKKPGASVKVSCKSSGYTFT

SEQ ID NO: 301 - protein, 32 aa, Homo sapiens
RVTMTRDTSISTAYMELSRLRSDDTAVYYCAR

SEQ ID NO: 302 - protein, 30 aa, Homo sapiens
QVQLVESGGGVVQPGRSLRLSCAASGFTFS

SEQ ID NO: 303 - protein, 33 aa, Homo sapiens
RFTISRDNSKNTLYLQMNSLRAEDTAVYYCARG

SEQ ID NO: 304 - protein, 30 aa, Homo sapiens
EVQLVESGGGLVQPGGSLRLSCAASGFTFS

SEQ ID NO: 305 - protein, 32 aa, Homo sapiens
RFTISRDNAKNSLYLQMNSLRAEDTAVYYCAR

SEQ ID NO: 306 - protein, 32 aa, Homo sapiens
RFTISRDNSKNTLFLQMNSLRAEDTAVYYCAR

SEQ ID NO: 307 - protein, 30 aa, Homo sapiens
QVQLVQSGAEVKKPGSSVKVSCKASGYTFT

SEQ ID NO: 308 - protein, 14 aa, Homo sapiens
WVRQAPGQGLEWIG

SEQ ID NO: 309 - protein, 32 aa, Homo sapiens
KATITADESTNTAYMELSSLRSEDTAFYFCAR

SEQ ID NO: 310 - protein, 32 aa, Homo sapiens
RFTISRDNSKNTLYLQMNRLRAEDTAVYYCAR

SEQ ID NO: 311 - protein, 30 aa, Homo sapiens
QVQLQESGPGLVKPSGTLSLTCAVSGGSIS

SEQ ID NO: 312 - protein, 14 aa, Homo sapiens
WVRQPPGKGLEWIG

SEQ ID NO: 313 - protein, 32 aa, Homo sapiens
RVTISVDKSKNQFSLKLSSVTAADTAVYYCAR

SEQ ID NO: 314 - protein, 32 aa, Homo sapiens
RFAISRDNAKNSLFLQMNSLRAEDTAVYYCAR

SEQ ID NO: 315 - protein, 32 aa, Homo sapiens
RFTISRDNARNSLYLQMNSLRAEDTAVYYCAR

SEQ ID NO: 316 - protein, 30 aa, Homo sapiens
EVQLVESGGGLVKPGGSLRLSCAASGFTFS

SEQ ID NO: 317 - protein, 14 aa, Homo sapiens
WVRQAPGKGLEWVG

SEQ ID NO: 318 - protein, 32 aa, Homo sapiens
RFTISRDDSKNTLYLQMNSLKAEDTAVYYCTT

SEQ ID NO: 319 - protein, 30 aa, Homo sapiens
QVQLQESGPGLVKPSQTLSLTCTVSGGSIS

SEQ ID NO: 320 - protein, 14 aa, Homo sapiens
WIRQLPGKGLEWIG

SEQ ID NO: 321 - protein, 32 aa, Homo sapiens
RVTISVDTSKKQFSLRLSSVTAADTAVYYCAR

SEQ ID NO: 322 - protein, 30 aa, Homo sapiens
EVQLVQSGAEVKKPGESLKISCKVSGYFFT

SEQ ID NO: 323 - protein, 14 aa, Homo sapiens
WVRQMPGKGLEYMG

SEQ ID NO: 324 - protein, 32 aa, Homo sapiens
QVTISADKSISTAYLQWSSLKASDTAMYYCAR

SEQ ID NO: 325 - protein, 32 aa, Homo sapiens
RVTMTTDTSTSTAYMELRSLRSDDTAVYFCAR

SEQ ID NO: 326 - protein, 30 aa, Homo sapiens
EVQLVQSGGGVVQPGRSLRLSCAASGFTFS

SEQ ID NO: 327 - protein, 30 aa, Homo sapiens
EVQLVQSGAEVKKPGASVKVSCKASGYTFT

SEQ ID NO: 328 - protein, 32 aa, Homo sapiens
RVTMTTDTSTSTAYMELRSLRSDDTAVYYCAR

SEQ ID NO: 329 - protein, 30 aa, Homo sapiens
EVQLVQSGAEVKKPGESLKISCKGSGYNFT

SEQ ID NO: 330 - protein, 14 aa, Homo sapiens
WVRQMPGKGLELMG

SEQ ID NO: 331 - protein, 32 aa, Homo sapiens
QVTISADKSISTAYLQWSSLKASDTAMYYCGS

SEQ ID NO: 332 - protein, 11 aa, Homo sapiens
WGRGTLVTVSS

SEQ ID NO: 333 - protein, 30 aa, Homo sapiens
QVTLKESGPALVKPTQTLTLTCTFSGFSLR

SEQ ID NO: 334 - protein, 32 aa, Homo sapiens
QLTISKDTSKNQVVLTMTNMDPVDTATYYCAR

SEQ ID NO: 335 - protein, 30 aa, Homo sapiens
QVQLQESGPGLVKPSETLSLTCTVSGGSVS

SEQ ID NO: 336 - protein, 14 aa, Homo sapiens
WIRQSPGKGLEWIG

SEQ ID NO: 337 - protein, 32 aa, Homo sapiens
RLTISIDTSKTQFSLKLSSVTAADTAIYYCVR

SEQ ID NO: 338 - protein, 32 aa, Homo sapiens
RFIISRDKSKNTLYLQMNSLRAEDTAVYYCAR

SEQ ID NO: 339 - protein, 32 aa, Homo sapiens
RVTMTRDTSISTAYMELSRLRSDDTAVYFCAR

SEQ ID NO: 340 - protein, 30 aa, Homo sapiens
EVQLVQSGGGLVQPGGSLRLSCTASGFTFS

SEQ ID NO: 341 - protein, 32 aa, Homo sapiens
RFTISRDNAKNTLYLQMNSLRAEDTAVYYCAR

SEQ ID NO: 342 - protein, 14 aa, Homo sapiens
WVRQAPGEGLEWVA

SEQ ID NO: 343 - protein, 30 aa, Homo sapiens
EVQLVESGGGVVQPGGSLRLSCAASGFTFS

SEQ ID NO: 344 - protein, 30 aa, Homo sapiens
QVQLVQSGAAVKKPGASVKVSCKASGYTFT

SEQ ID NO: 345 - protein, 32 aa, Homo sapiens
RVTMTRDTSISTASMELSRLRSDDTAVYFCAR

SEQ ID NO: 346 - protein, 32 aa, Homo sapiens
RFTISRDNAKNSLYLQMNSLRAEDTAVYYCTE

SEQ ID NO: 347 - protein, 30 aa, Homo sapiens
QVQLVQSGTEVKKPGASMKVSCKASGYTFT

SEQ ID NO: 348 - protein, 32 aa, Homo sapiens
RVTMTRDTSTNTVYMELSSLRSEDTAMYYCAR

SEQ ID NO: 349 - protein, 32 aa, Homo sapiens
RFTISRDNAKNSLYLQMNSLRDEDTAVYYCAR

SEQ ID NO: 350 - protein, 441 aa, Homo sapiens
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 351 - protein, 215 aa, Homo sapiens
ESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 352 - protein, 441 aa, Homo sapiens
EVQLVQSGAEVKKPGASVKVSCKASGYTLTSYGISWVRQAPGQGLEWMGWVSFYNGNTNYAQKLQGRGTMTRDPSTSTAYMELSRLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 353 - protein, 441 aa, Homo sapiens
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKKLSLSPGK

SEQ ID NO: 354 - protein, 441 aa, Homo sapiens
EVQLVQSGAEVKKPGASVKVSCKASGYTLTSYGISWVRQAPGQGLEWMGWVSFYNGNTNYAQKLQGRGTMTRDPSTSTAYMELSRLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKKLSLSPGK

SEQ ID NO: 355 - protein, 220 aa, Homo sapiens
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 446 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 357 - protein, 446 aa, Homo sapiens
QVQLVQSGAEVKKPGASVKVSCKASGYTFTSYGISWVRQAPGQGLEWMGWISAYNGNTNYAQKLQGRVTMTRDTSTSTAYMELSRLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 358 - protein, 446 aa, Homo sapiens
QVQLVQSGAEVKKPGASVKVSCKASGYTFTSYGISWVRQAPGQGLEWMGWIKAYNGNTNYAQKLQGRVTMTRDTSTSTAYMELSRLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 359 - protein, 450 aa, Homo sapiens
QVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 214 aa, Homo sapiens
EIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 361 - protein, 450 aa, Homo sapiens
EVQLVESGGGVVQPGGSLRLSCAASGFTFSNYGMHWVRQAPGEGLEWVAAIWFDASDKYYADAVKGRFTISRDNAKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 450 aa, Homo sapiens; identical to SEQ ID NO: 359

SEQ ID NO: 363 - protein, 214 aa, Homo sapiens
EIVMTQSPATLSVSPGERATLSCRASQSVSSNLAWYQQKPGQAPRLLIYGAATRATGIPDRVSGSGSGTEFTLTISRLEPEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 450 aa, Homo sapiens; identical to SEQ ID NO: 361

SEQ ID NO: 365 - protein, 214 aa, Homo sapiens; identical to SEQ ID NO: 363

SEQ ID NO: 366 - protein, 441 aa, Homo sapiens; identical to SEQ ID NO: 353

SEQ ID NO: 367 - protein, 214 aa, Homo sapiens; identical to SEQ ID NO: 360

SEQ ID NO: 368 - protein, 450 aa, Homo sapiens
QVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 369 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 370 - protein, 14 aa, Homo sapiens
TGTSSDVGGYNSVS

SEQ ID NO: 371 - protein, 7 aa, Homo sapiens
EVSNRPS

SEQ ID NO: 372 - protein, 9 aa, Homo sapiens
NSYTSTSMV

SEQ ID NO: 373 - protein, 10 aa, Homo sapiens
GYTLTSYGIS

SEQ ID NO: 374 - protein, 16 aa, Homo sapiens
WVSFYNGNTNYAQKLQ

SEQ ID NO: 375 - protein, 6 aa, Homo sapiens
GYGMDV

SEQ ID NO: 376 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 377 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 378 - protein, 115 aa, Homo sapiens; identical to SEQ ID NO: 376

SEQ ID NO: 379 - protein, 109 aa, Homo sapiens; identical to SEQ ID NO: 377

SEQ ID NO: 380 - protein, 4 aa, Homo sapiens
KKKP

SEQ ID NO: 381 - protein, 4 aa, Homo sapiens
PGKP

SEQ ID NO: 382 - protein, 5 aa, Homo sapiens
PGKKP

SEQ ID NO: 383 - protein, 6 aa, Homo sapiens
PGKKKP

(19) United States
(12) Patent Application Publication
(10) Pub. No.: US 2024/0343832 A1
(43) Pub. Date: Oct. 17, 2024

(54) LOW-VISCOSITY ANTIGEN BINDING PROTEINS AND METHODS OF MAKING THEM

(71) Applicant: AMGEN INC., THOUSAND OAKS, CA (US)

(72) Inventors: JOON HOI HUH, CULVER CITY, CA (US); RIKI STEVENSON, VENTURA, CA (US); PAVEL BONDARENKO, THOUSAND OAKS, CA (US); ANDREW NICHOLS, CALABASAS, CA (US); DA REN, THOUSAND OAKS, CA (US); NEERAJ JAGDISH AGRAWAL, NATICK, MA (US); RICHARD SMITH, BELMONT, CA (US)

(73) Assignee: AMGEN INC., THOUSAND OAKS, CA (US)

(21) Appl. No.: 18/644,757

(22) Filed: Apr. 24, 2024

Related U.S. Application Data
(62) Division of application No. 17/346,156, filed on Jun. 11, 2021, now Pat. No. 11,993,663.
Application No. 17/346,156 is a division of application No. 16/338,292, filed on Mar. 29, 2019, now Pat. No. 11,059,908.
(60) Provisional application No. 62/546,469, filed on Aug. 16, 2017.
(60) Provisional application No. 62/430,773, filed on Dec. 6, 2016.
(60) Provisional application No. 62/401,770, filed on Sep. 29, 2016.

(51) Int. Cl. C07K 16/40; C07K 16/28; G01N 11/14

(52) U.S. Cl. CPC C07K 16/40; C07K 16/2869; C07K 2317/21; C07K 2317/24; C07K 2317/52; C07K 2317/55; C07K 2317/90; C07K 2317/92; C07K 2317/94; G01N 11/14

(57) ABSTRACT

The present invention concerns a method for preparing antigen binding proteins specific for PCSK9 with reduced viscosity. The method proceeds by replacing residues in high viscosity variable domain subfamilies with residues in correlating low viscosity subfamilies. The method further comprises substituting residues in the Fc domain with residues associated with low viscosity and adding charged residues to the C-terminus of the Fc domain. The present invention further concerns antigen binding proteins produced by this method.

### CROSS REFERENCES TO RELATED APPLICATIONS

This application is divisional of U.S. Ser. No. 16/338,292, filed Mar. 29, 2019, which is a national stage application under 35 U.S.C. § 371 of International Application No. PCT/US17/53967, having an international filing date of Sep. 28, 2017, which claims the priority of U.S. Provisional Application No. 62/546,469, filed on Aug. 16, 2017; U.S. Provisional Application No. 62/430,773, filed on Dec. 6, 2016; and U.S. Provisional Application No. 62/401,770, filed Sep. 29, 2016, each of which is hereby incorporated by reference in its entirety for all purposes.

### FIELD OF THE INVENTION

This invention relates to biopharmaceuticals, particularly to therapeutic antigen binding proteins, methods of use thereof, pharmaceutical compositions thereof, and processes of making them. In particular, this invention relates to antigen binding proteins, particularly antibodies, mutated to reduce viscosity.

### BRIEF DESCRIPTION OF THE SEQUENCE LISTING

Incorporated herein by reference in its entirety is a Sequence Listing entitled, “A-2063-US-PSP3_SeqList_ST25.txt”, comprising SEQ ID NO:1 through SEQ ID NO:379, which includes nucleic acid and/or amino acid sequences disclosed herein. The Sequence Listing has been submitted herein in ASCII text format via EFS, and thus constitutes both the paper and computer readable form thereof. The Sequence Listing was first created using PatentIn on Aug. 16, 2017, and is 4.32 MB in size.

### BACKGROUND OF THE INVENTION

Currently, monoclonal antibodies (mAbs) are the most popular modality of modern therapeutic proteins on the market and under development. The differences between antibodies are predominantly in the antigen binding domains or complementary determining regions (CDRs). These differences in the CDRs are thought to result in differences in transient protein-protein interaction propensity that manifest themselves as bulk solution viscosity. Several groups have described the presence of reversible clusters of antibodies in viscous antibody solutions (predominantly dimers). Several theoretical descriptions of polymer viscosity have been proposed to explain the interactions of these clusters as a mechanism for bulk solution viscosity behavior.

Antibodies usually work as antagonists and, therefore, large amounts, often exceeding 100 mg per dose, are required to block undesirable interactions. For patient comfort, a single subcutaneous injection of a 1 mL volume is the most preferred mode of administration. The need to administer large amounts of mAbs in a relatively small volume has required high concentration formulations at or exceeding 100 mg/ml. Antibodies are large biopolymers with molecular weights of about 150 kDa, and their high concentrations result in high sheer stress and high viscosity due to protein-protein and protein-wall interactions during filtration and passage through the injection needles and in subcutaneous space. High viscosity presents challenges in the manufacture of therapeutic antigen binding proteins as well as in their administration to patients, including prohibitively high back pressure during injections leading to malfunction of injections devices, difficulty of manual administration, decreased bio-availability and patient discomfort.

The development and use of high concentration therapeutic monoclonal antibody solutions has accelerated as the cost of biopharmaceutical production has decreased. In some cases, these antibody solutions possess viscous solution attributes that can make manufacturing and administration of the intended dose challenging. The differences in the CDRs that appear to determine if an antibody is “viscous” or “not viscous” are likely related to the propensity of the CDRs to drive protein-protein interaction.

Significant efforts are underway in the industry to understand the nature of interactions leading to high viscosity and to reduce the viscosity of high viscosity antibody formulations. The most important parameters affecting viscosity of the antibody formulations include:
intermolecular interactions defined by the pI of the protein and the pH of the solution. Cheng et al. (2013), “Linking the solution viscosity of an IgG2 monoclonal antibody to its structure as a function of pH and temperature,” J. Pharm Sci. 102:4291-4304.
Charge interactions. Yadav et al. (2012), “Viscosity behavior of high-concentration monoclonal antibody solutions: correlation with interaction parameter and electroviscous effects,” J. Pharm Sci. 101:998-1011; Yadav et al. (2012), “The influence of charge distribution on self-association and viscosity behavior of monoclonal antibody solutions.” Mol Pharm 9 (4): 791-802; Singh et al. (2014), “Dipole-Dipole Interaction in Antibody Solutions: Correlation with Viscosity Behavior at High Concentration,” Pharm Res. 31 (9): 2549-2558; Chaudhri et al. (2013), “The role of amino acid sequence in the self-association of therapeutic monoclonal antibodies: insights from coarse-grained modeling,” J. Phys. Chem. B 117:1269-1279.
Hydrophobic interactions. Guo et al. (2012), “Structure-activity relationship for hydrophobic salts as viscosity-lowering excipients for concentrated solutions of monoclonal antibodies,” Pharm Res 29:3102-3109.

The highest solution viscosity was observed under conditions with the most negative diffusion interaction parameter kD, the highest apparent radius and the lowest net charge. Neergaard et al. (2013), “Viscosity of high concentration protein formulations of monoclonal antibodies of the IgG1 and IgG4 subclass-prediction of viscosity through protein-protein interaction measurements,” Eur. J. Pharm Sci. 49:400-410. The diffusion interaction parameter (kD), a component of the osmotic second virial coefficient (B (2)) correlated well (R>0.9) with the viscosity of concentrated mAb solutions, while the mAb net charge correlated weakly (R<0.6), indicating that weak intermolecular interactions are important in governing the viscoelastic behavior of concentrated mAb solutions. Connolly, et al. (2012), “Weak interactions govern the viscosity of concentrated antibody solutions: high-throughput analysis using the diffusion interaction parameter,” Biophys. J. 103:69-78. In a study reported in this specification, primary sequences linked to 3D structure were utilized. See Honegger et al. (2001), “Yet another numbering scheme for immunoglobulin variable domains: an automatic modeling and analysis tool,” J. Mol. Biol. 309:657-670. Viscosity values of several mAb molecules were measured to develop a model for viscosity prediction of mAbs via machine learning algorithms. Structural position, charge and hydrophobicity were the main parameters of amino acids utilized for the model.

Viscosity of monoclonal antibodies was assessed using molecular information in the following articles: Li, L. et al. (2014), “Concentration dependent viscosity of monoclonal antibody solutions: explaining experimental behavior in terms of molecular properties,” Pharm. Res. 31:3161-3178; and Sharma et al. (2014), “In silico selection of therapeutic antibodies for development: viscosity, clearance, and chemical stability,” Proc. Natl. Acad. Sci. U.S.A. 111:18601-6.

The net result of the interactions between antibodies is either an extended transient network of interactions (a percolating network) that result in a viscous solution or the formation of larger oligomers that then somehow influence the solution rheology as larger structures. In studies reported in this specification, a small number of viscous antibodies was used as the subject for biochemical and biophysical analysis in an attempt to deduce specific protein-protein interactions that might lead to a viscous antibody solution.

The Aho numbering approach was utilized in the past to improve stability and other biophysical properties. Ewert et al. (2003), “Structure-based improvement of the biophysical properties of immunoglobulin VH domains with a generalizable approach,” Biochemistry 42:1517-1528; Ewert et al. (2003), “Biophysical properties of human antibody variable domains,” J. Mol. Biol. 325:531-553; Ewert et al. (2004), “Stability improvement of antibodies for extracellular and intracellular applications: CDR grafting to stable frameworks and structure-based framework engineering,” Methods 34:184-199; and Rothlisberger et al. (2005), “Domain interactions in the Fab fragment: a comparative evaluation of the single-chain Fv and Fab format engineered with variable domains of different stability,” J Mol. Biol. 347:773-789. The Aho numbering system was also utilized in the past to reduce propensity for aggregaton. Borras et al. (2013), U.S. Pat. No. 8,545,849.

### SUMMARY OF THE INVENTION

This invention relates to methods for reducing viscosity of antigen binding proteins by modifying sequences in framework regions and/or the Fc domain that are shown to be associated with high viscosity.

In the process details which follow, all variable region amino acids are identified by Aho numbering, all amino acids from conserved regions are identified by EU numbering. Aho numbering is aligned and correlated with the other main numbering schemes including EU (Edelman et al. (1969), “The covalent structure of an entire gamma immunoglobulin molecule,” Proc. Natl. Acad. Sci. U.S.A. 63, 78-85), Kabat (Kabat et al. (1991), Sequences of proteins of immunological interest, Fifth Edition. NIH Publication No. 91-3242), Chothia (Chothia et al., (1992), “Structural repertoire of the human VH segments,” J. Mol. Biol. 227:799-817); (Tomlinson et al., (1995), “The structural repertoire of the human V kappa domain,” EMBO J 14:4628-4638). Any of the four numbering systems can be interchangeably used to identify the preferred amino acid substitutions described in this specification.

If the antigen binding protein comprises the VH1|1-18 germline subfamily, the method comprises modifying the VH1 sequence to comprise one or more substitutions selected from 82X¹, 94X², and 95X³, wherein X¹ is a basic residues (R, K or H), X² is a polar, uncharged residue (S, T, N or Q) and X³ is a basic residue (R, K, or H). All residues are identified by the Aho numbering system. Preferred mutations for the VH1|1-18 germline subfamily are 82R, 94S, and 95R. The method as applied to the VH1|1-18 germline subfamily may further comprise substitution 59X²⁰ wherein X²⁰ is a basic residue (R, K or H), with the mutation 59K preferred.

If the antigen binding protein comprises the VH3|3-33 germline subfamily, the method comprises modifying the VH3 sequence to comprise one or more substitutions selected from 1X⁴, 17X⁵, and 85X⁶, wherein X⁴ is a charged negative residue (D or E), X⁵ is a small hydrophobic residue (G, A, V, I, L, or M), and X⁶ is a small hydrophobic residue (G.A, V, I, L, or M). All residues are identified by the Aho numbering system. Preferred mutations for the VH3|3-33 germline subfamily are 1E, 17G, and 85A.

If the antigen binding protein comprises the VK3|L16 germline subfamily, the method comprises modifying the VK3 sequence to comprise one or more substitutions selected from 4X¹⁰, 13X¹¹, 76X¹², 78F, 95X¹³, 97X¹⁴, and 98P, wherein X¹⁰ is selected from G, A, V, I, L, and M, X¹¹ is selected from G, A, V, I, L, and M, X¹² is selected from D and E, X¹³ is selected from R, K and H, and X¹⁴ is selected from D and E. All residues are identified by the Aho numbering system. Preferred mutations for the VK3|L16 germline subfamily are 4L, 13L, 76D, 95R, 97E, and 98P.

If the antigen binding protein comprises the VK3|L6 germline subfamily, the method comprises modifying the VK3 sequence to comprise one or more substitutions selected from 76X¹² and 95X¹³. Preferred mutations for the VK3|L6 germline subfamily are 76D and 95R.

The methods of this invention further comprise modifying the Fc domain to comprise one or more substitutions selected from 253X¹⁵, 440X¹⁶, and 439X¹⁷, wherein X¹⁵ is a small hydrophobic residue (G, A, V, I, L, or M), X¹⁶ is a basic residue (R, K, or H), and X¹⁷ is a charged negative residue (D or E), wherein the Fc domain sequence comprises only one of 440X¹⁶ and 439X¹⁷. All residues are identified by the EU numbering system. Preferred mutations of the Fc domain are 253A, 440K, and 439E.

The methods of this invention further comprise modifying the C-terminus of the Fc domain sequence to comprise X¹⁸X¹⁹ wherein X¹⁸ is one to four amino acids selected from D and E or from H, K, and R, and X¹⁹ is selected from P, M, G, A, V, I, L, S, T, N, Q, F, Y and W and is absent when X¹⁸ comprises D or E, is present when X¹⁸ comprises K or R at its C-terminal end, and is present or absent when X¹⁸ comprises H at its C-terminal end. Preferred Fc C-terminal modifications comprise KP, KKP, KKKP, E or EE at the C-terminus.

The foregoing methods are preferably applied to the high viscosity antibodies shown in FIGS. 1A and 1B hereinafter. The part of the method involving the VH1|1-18 sequence is preferably applied to antibodies AF, AK, AL, AN, and AO from FIG. 1B. The part of the method involving the VH3|3-33 sequence is preferably applied to antibodies AQ, AM, AI, and AG from FIG. 1B. The part of the method involving the VK3|L16 sequence is preferably applied to antibodies AF and AQ from FIG. 1B. The part of the method involving the VK3|L6 sequence is preferably applied to antibody AJ.

The methods of this invention further comprise a method of preparing an antigen binding protein that reaches maximum serum concentration faster than does a parental antibody when the antigen binding protein and the parental antibody are administered at the same concentration, which comprises introducing sequence modification 440X¹⁶ in the parental antibody wherein X¹⁶ is selected from R, K, and H. In a preferred method, the sequence-modified antigen binding protein reaches maximum serum concentration after subcutaneous injection at least twice as fast as the parental antibody. Also within this invention is a method of preparing an antigen binding protein that reaches a maximum serum concentration after subcutaneous injection that is higher than that of a parental antibody when the antigen binding protein and the parental antibody are administered at the same concentration, which comprises introducing sequence modification 440X¹⁶ in the parental antibody wherein X¹⁶ is selected from R, K, and H. In a preferred method, the sequence-modified antigen binding protein reaches a maximum serum concentration that is at least about 25% higher than that of the parental antibody. In each of these methods, the preferred X¹⁶ is K and the preferred parental antibody is a PCSK9 polypeptide (antibody AK most preferred).

The invention further relates to a mutant antigen binding protein, which comprises one or more sequences selected from:
a. a VH1|1-18 germline subfamily sequence comprising one or more substitutions selected from 82X¹, 94X², and 95X³, wherein X¹ is selected from R, K and H, X² is selected from S, T, N and Q and X³ is selected from R, K, and H;
b. a VH3|3-33 germline subfamily sequence comprising one or more substitutions selected from 1X⁴, 17X⁵, and 85X⁶, wherein X⁴ is selected from D and E, X⁵ is selected from G, A, V, I, L, and M, and X⁶ is selected from G.A, V, I, L, and M;
c. a VK3|L16 germline subfamily, comprising one or more substitutions selected from 4X¹⁰, 13X¹¹, 76X¹², 78F, 95X¹³, 97X¹⁴, and 98P, wherein X¹⁰ is selected from G, A, V, I, L, and M, X¹¹ is selected from G, A, V, I, L, and M, X¹² is selected from D and E, X¹³ is selected from R, K and H, and X¹⁴ is selected from D and E, wherein the mutant antigen binding protein does not comprise only substitution 78F;
d. a VK3|IL6 germline subfamily, comprising one or more substitutions selected from 76X¹² and 95X¹³;
e. an Fc domain sequence comprising one or more substitutions selected from 253X¹⁰, 440X¹¹, and 439X¹², wherein X¹⁰ is selected from G, A, V, I, L, and M, X¹¹ is selected from R, K, and H, and X¹² is selected from D and E, wherein the antigen binding protein comprises at least one of 253X¹⁵ or modifications selected from subparagraphs a, b, c, d and f when X¹⁶ is K and X¹⁷ is E and the antigen binding protein specifically binds CD20; and
f. an Fc domain sequence comprising at the C-terminus X¹⁸X¹⁹ wherein X¹⁸ is one to four amino acids selected from D and E or from H, K, and R, and X¹⁹ is selected from P, M, G, A, V, I, L, S, T, N, Q, F, Y and W and is absent when X¹⁸ comprises D or E, is present when X¹⁸ comprises K or R at its C-terminal end, and is present or absent when X¹⁸ comprises H at its C-terminal end, and wherein the antigen binding protein comprises at least one of 253X¹⁵ or substitutions selected from subparagraphs a through e when PGKP, PGKKP, PGKKKP, or PGE appears at the C-terminus and the antigen binding protein specifically binds CD20 or CD38. Preferred Fc C-terminal modifications comprise KP, KKP, KKKP, E or EE at the C-terminus;
   wherein the variable region amino acids are numbered according to the Aho numbering system and all amino acids from conserved regions including Fc are according to EU numbering.

Preferred antigen binding proteins in accordance with this invention are those wherein the foregoing modifications are applied to antibodies of FIGS. 1A and 1B hereinafter. Also preferred are antigen binding proteins wherein:
the VH1|1-18 germline subfamily sequence comprises one or more substitutions selected from 82R, 94S, and 95R, with antigen binding proteins having all such substitutions most preferred;
the VH3|3-33 germline subfamily sequence comprises one or more of substitutions 1E, 17G, and 85A, with antigen binding proteins having all such substitutions most preferred;
the VK3|L16 germline subfamily sequence comprises one or more substitutions selected from 4L, 13L, 76D, 95R, 97E, and 98P, with antigen binding proteins having all such substitutions most preferred;
the VK3|L6 germline subfamily sequence comprises one or more substitutions selected from 76D and 95R, with antigen binding proteins having both such substitutions most preferred;
the Fc domain sequence comprises one or more substitutions selected from 253A, 440K, and 439E, with antigen binding proteins having all such substitutions most preferred; and
the Fc domain C-terminus comprises a sequence selected from KP, KKP, KKKP, and E.

All of the foregoing preferred amino acid substitutions in variable regions are identified by the Aho numbering system. All residues in conserved regions including Fc are identified by the EU numbering system.

Preferred antigen binding proteins in accordance with this invention include: antibodies AF, AK, AL, AN and AO from FIG. 1B having one or more, most preferably all, of the foregoing VH1|1-18 germline subfamily substitutions; antibodies AQ, AM, AI, and AG from FIG. 1B having one or more, most preferably all, of the VH3|3-33 germline subfamily substitutions; antibodies AF and AQ having one or more, most preferably all, of the VK3|L16 germline subfamily substitutions; antibody AJ from FIG. 1B having one or more, most preferably all, of the VK3|L6 germline subfamily substitutions; and antibodies BA, AH, and AN from FIG. 1B having one or more, preferably all, of the Fc substitutions noted above.

Due to the foregoing sequence modifications, the invention further relates to antigen binding proteins that specifically bind to PCSK9 comprising a heavy chain sequence selected from SEQ ID NOS: 352, 353, 354, 366, and 368, preferably also comprising a light chain sequence of SEQ ID NO: 351.

Due to the foregoing sequence modifications, the invention also relates to antigen binding proteins that specifically bind c-fms comprising a heavy chain sequence selected from SEQ ID NOS: 356, 357, and 358, preferably further comprising a light chain sequence of SEQ ID NO: 355.

Due to the foregoing sequence modifications, the invention also relates to antigen binding proteins that specifically bind GIPR comprising a heavy chain sequence selected from SEQ ID NOS: 359, 361, 362, 364, and 368, preferably further comprising a light chain sequence selected from SEQ ID NOS: 360, 363, 365, and 367.

All modified antigen binding proteins are useful for the same indications as described previously for the unmodified antibodies.

Each of the antigen binding proteins from FIGS. 1A and 1B having mutated heavy chains is preferred to further comprise a light chain sequence as noted in the unmodified parent antibody of FIGS. 1A and 1B. Each of the foregoing antigen binding proteins having a mutated light chain is preferred to further comprise a heavy chain sequence as noted above or as appearing in the unmodified parent antibody of FIGS. 1A and 1B.

The invention further comprises antigen binding proteins as described above that have improved pharmacokinetic properties. The invention comprises an antigen binding protein optionally having any of the aforementioned sequence modifications wherein:
a. the antigen binding protein comprises the sequence modification 440X¹⁶ relative to a parental antibody lacking the 440X¹⁶ sequence modification,
b. the antigen binding protein reaches maximum serum concentration after subcutaneous injection faster than does the parental antibody when the antigen binding protein and the parental antibody are administered at the same concentration, and
c. the antigen binding protein reaches a maximum serum concentration after subcutaneous injection that is higher than that of the parental antibody when the antigen binding protein and the parental antibody are administered at the same concentration.

The preferred parental antibody for such an antigen binding protein is a PCSK9 binding polypeptide, with antibody AK most preferred. The preferred substituent X¹⁶ in such an antigen binding protein is K. Further within this invention is a method of treating hypercholesterolemia with such an antigen binding protein.

The invention also relates to isolated nucleic acids encoding the antigen binding proteins of the invention, as well as vectors comprising the nucleic acids, host cells comprising the vectors, and methods of making and using the antigen binding proteins.

In other embodiments, the present invention provides compositions comprising the antigen binding proteins and kits comprising the antigen binding proteins, as well as articles of manufacture comprising the antigen binding proteins.

### BRIEF DESCRIPTION OF THE FIGURES

FIGS. 1A and 1B show a table of viscosity values measured for IgG1 and IgG2 monoclonal antibodies formulated at 150 mg/mL in a formulation buffer including 20 mM acetate and 9% sucrose at pH 5.2 (without polysorbate). FIGS. 1A and 1B show the targets of the antibodies studied as well as their light and heavy chain types, germline subfamilies, concentration, pI, and viscosity. Each antibody in FIGS. 1A and 1B has the amino acid sequences as noted in the figures and the Sequence Listing. The heavy and light chain amino acid sequences are encoded by nucleic acids having the SEQ ID NOS immediately preceding them in the Sequence Listing.

FIGS. 1C and 1D show the sequence identification numbers (SEQ ID NOS) for the framework regions and Fc regions of the antibodies of FIGS. 1A and 1B. FIG. 1D also shows antibody BA, which is discussed in FIG. 17.

FIG. 2 shows high viscosity and low viscosity subtype pairs identified in this specification.

FIG. 3 shows expression results for AK and AO antibody molecules and their mutants, including relative values for titer, final viable cell density (VCD) and viability at harvest after 7 days of cell culture. AK values are at 100%.

FIG. 4 shows potency of AK parent antibodies (AK control, AK) and their mutants.

FIG. 5 shows viscosity of mutants relative to the parent AK and AO antibodies. Average viscosity values of high viscosity VH1|1-18 and low viscosity VH1|1-02 from the set of 43 antibodies are also shown for comparison.

FIG. 6 shows measured viscosity values of mAbs of the high viscosity VH1|1-18 and the low viscosity VH1|1-02 subtypes versus calculated whole molecule pI values. The low-viscosity mutants of AK and AO antibodies are also shown.

FIG. 7 shows measured viscosity values of mAbs of the high viscosity VH3|3-33 and the low viscosity VH3|3-07 subtypes versus calculated whole molecule pI. The low-viscosity mutant AQ (1, 17, 85) is also shown.

FIG. 8 shows measured viscosity values of mAbs with high viscosity VK3|L16 and VK3|L6 subfamilies and the low viscosity VK3|A27 subfamily versus calculated whole molecule pI values. The low-viscosity mutant AQ (4 13 76 95 97 98) is also shown.

FIG. 9 is a table showing global sequence parameters of mAbs high viscosity VH1|1-18 and low viscosity VH1|1-02 subtypes (germlines). The mAbs in FIG. 9 are sorted by viscosity. The table includes their mAb symbols, measured viscosity values, calculated pI values, and VL and VH germlines. For VH germlines, higher viscosity VH1|1-18 are shown in bold and underlined. Heavy chain framework 3 sequences are shown. Residues correlating with high viscosity are in bold and underlined. VBase sequences are added for VH1|1-18 and VH1|1-02 germline subfamilies for comparison to illustrate that the different residues are typical residues for the subfamilies.

FIG. 10 is a table showing produced and characterized mutants of the AK and AO antibodies.

FIG. 11 is a table showing global sequence parameters of thirteen mAbs with VH3 heavy chains. FIG. 11 includes the mAb symbols, measured viscosity values, calculated pI values, HC and LC types, and VL and VH subtypes (germlines). Higher viscosity VH3|3-33 subfamily is shown in bold and underlined. Residues correlating with high viscosity are bold and underlined. VBase sequences are added for VH3|3-33 and VH3|3-07 germline subfamilies for comparison to illustrate that the different residues are typical for those subfamilies.

FIG. 12 is a table showing global sequence parameters of fourteen mAbs with VK3 light chains. The mAbs in FIG. 12 are sorted by viscosity, including their mAb symbols, measured viscosity values, calculated pI values, HC and LC types, and VL and VH subtypes (germlines). Higher viscosity VK3|L16 and VK3|L6 subfamilies are in bold and underlined. Light chain residues that are consistently different between VK3|L16 and VK3|L6 subfamilies as compared to the VK3|A27 subfamily are shown on the right side. Residues correlating with high viscosity in the VK3|L16 and VK3|L6 subfamilies are bold and underlined. VBase sequences are added for VK3|L16 and VK3|A27 germline subfamilies for comparison to illustrate that the different residues are typical residues for the subfamilies.

FIGS. 13A and 13B show average values for measured viscosity and calculated pI values for intact antibody molecules containing the specified heavy chain and light chain germline families and germline subfamilies of VH1, VH3 and VK3. The X-axis contains families and the number of members in each family and subfamily.

FIG. 14A shows that mutations in the Fc-Fc interaction surface can decrease solution viscosity. The viscosity of concentrations of antibody AK and antibody AK mutants I253A and S440K are shown.

FIG. 14B shows that a double mutant that restores wild type complement activity also restores wild type viscosity. The figure shows the viscosity of concentrations of antibody AK, antibody AK mutant I253A, antibody AK mutant S440K, and antibody AK double mutant K439E/S440K. The figure also shows the viscosity of antibody AK mutant K439E, antibody AK mutant H433A, and antibody AK mutant N434A which do not decrease viscosity relative to the antibody AK parent.

FIG. 15 is a table showing the absolute and relative viscosity values of parent antibody AK and various mutants. The parent antibody AK and the mutants are used in a nonhuman primate study of the pharmacokinetics and pharmacodynamics of antibody AK and low viscosity mutants.

FIG. 16 is a schematic of EDC chemical cross-linking (see Example 2).

FIG. 17 is a table showing the viscosity of proteins selected for Fc mutations to lower viscosity variants.

FIG. 18 shows concentration-dependent formation of antibody system oligomers by EDC chemical cross-linking of antibody AH.

FIG. 19A shows that a S440K mutation in the Fc region reduces viscosity in antibody AQ. (Note that the concentration of the mutant is actually 150 mg/mL.) FIG. 19B is a scatter plot of the same data with an exponential fit. The diamonds in FIG. 19B denote the unmodified antibody AQ at the concentrations shown and the square shows the S440K mutant at 150 mg/mL.

FIG. 20A is a table showing produced and characterized mutants of antibody AQ, including measured concentration and viscosity.

FIG. 20B shows cAMP response of 293/huGIPR cells expressing human GIP receptors activated by GIP and blocked by the anti-GIPR antibodies. The in vitro cAMP activity was equally unaffected by viscosity mutations. The potency remained the same within the error margin of the assay.

FIG. 21 shows a summary of the experimental design for a single-dose subcutaneous bolus pharmacokinetic study in male cynomolgus monkeys as described in further detail in a working example hereinafter.

FIG. 22 shows mean pharmacokinetic parameter estimates of antibody AK or low viscosity mutant homologues after subcutaneous administration of 10 mg/kg to male cynomolgus monkeys (N=4 males). Introduction of a mutation in the Fc region that reduces viscosity reduces Tmax and increases Cmax.

FIG. 23 shows the percentage of LDL-C compared to pretest (Day CLAB). * Percent change is expressed as the individual animal post-dose value divided by the Day 1 pretest value. All four antibodies (parent antibody AK, AK Fc mutant, AK Fab mutant and AK double Fc/Fab mutant) all induce reductions in LDL-C.

FIGS. 24A and 24B show a pharmacokinetic profile (μg/mL) with corresponding low density lipoprotein (LDL) concentration (mg/dL) profile in plasma. LDL concentrations and test article serum concentrations are presented as mean values from four animals. Solid circles with a solid line indicate the serum concentrations of the parent antibody AK. Open squares with a dashed line indicate the serum concentrations of the antibody bearing the Fab mutation. Solid triangles with a solid line indicate the serum concentrations of the antibody bearing the Fc mutation. Open diamonds with a dashed line indicate the serum concentrations of the antibody bearing both the Fab and Fc mutations. These data indicate that presence of a mutation in the Fc region that reduces viscosity of the antibody formulation results in a reduced time to Tmax and a higher Cmax. All the mutant forms of the parent antibody retain the ability to lower serum LDL-C (lower panel).

### DETAILED DESCRIPTION OF THE INVENTION

### Definition of Terms

In the description that follows, a number of terms are used extensively. The following definitions are provided to facilitate understanding of the invention.

Unless otherwise specified, “a”, “an”, “the”, and “at least one” are used interchangeably and mean one or more than one.

“Antigen binding protein” refers to a protein or polypeptide that comprises an antigen-binding region or antigen-binding portion that has a strong affinity for another molecule to which it binds (antigen). Antigen-binding proteins encompass antibodies, peptibodies, antibody fragments (e.g., Fab, Fab′, F(ab′)₂, Fv, single domain antibody), antibody derivatives, antibody analogs, fusion proteins, and antigen receptors including chimeric antigen receptors (CARs).

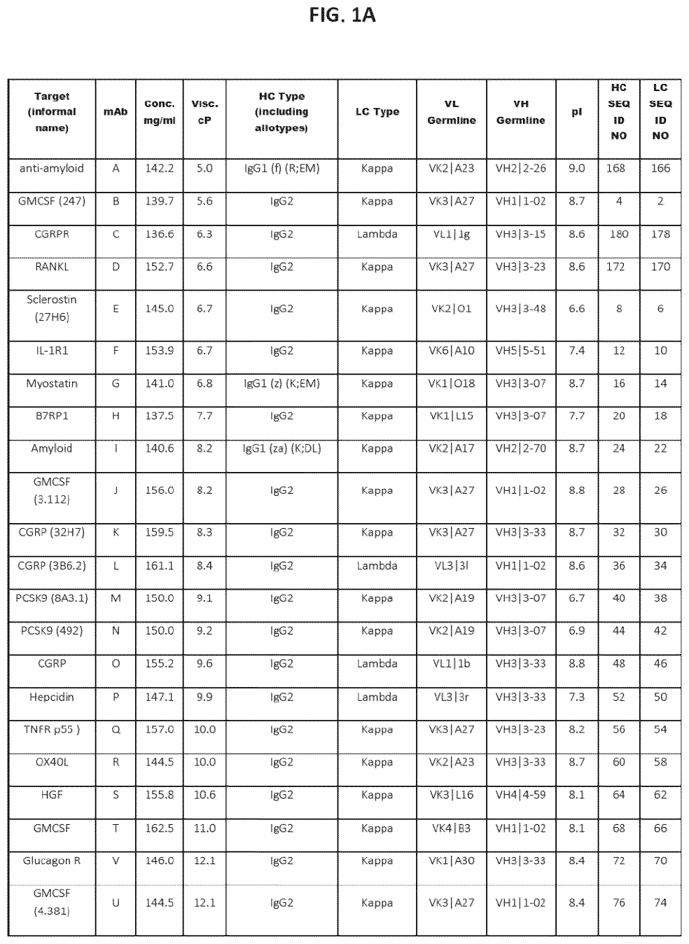

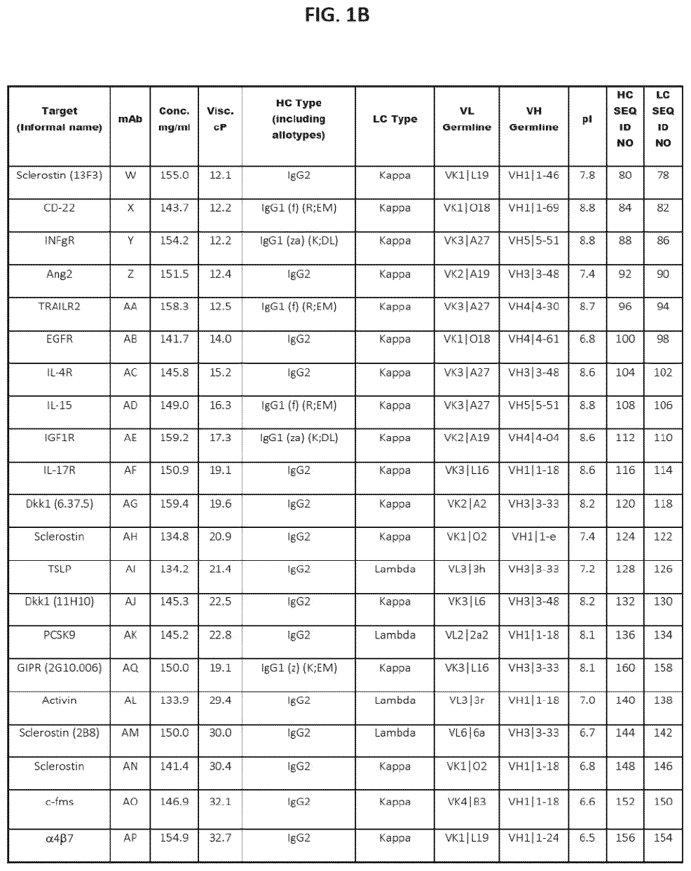

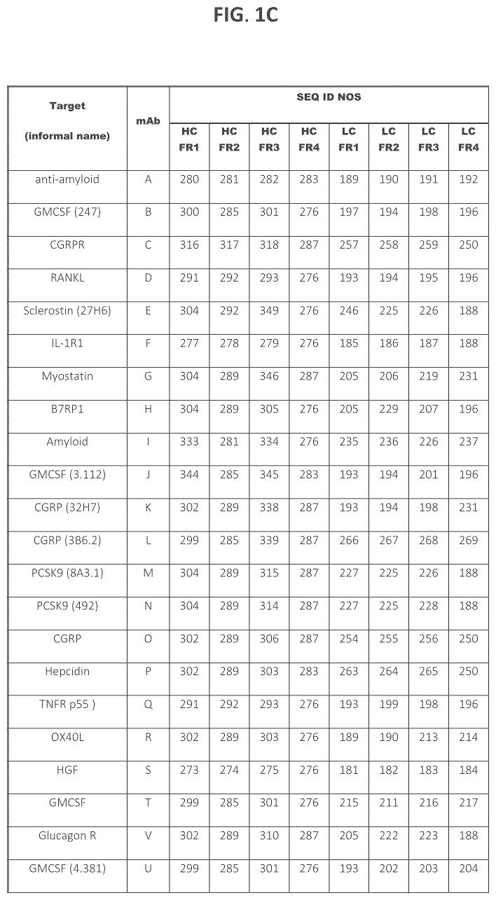

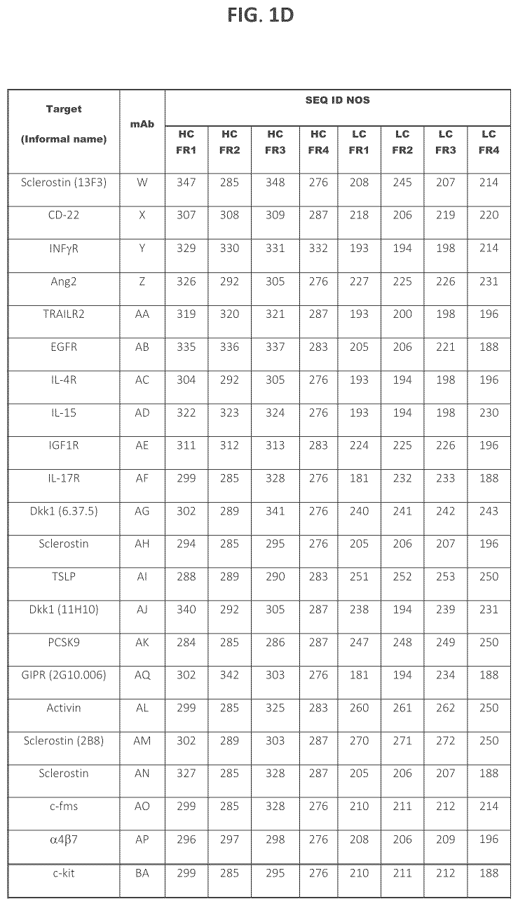

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. Thus, as used herein, the term “antibody” or “antibody peptide(s)” refers to an intact antibody, an antibody that competes for specific binding with an antibody disclosed in this specification, or an antigen-binding fragment (e.g., Fab, Fab′, F(ab′)₂, Fv, single domain antibody) thereof that competes with the intact antibody for specific binding and includes chimeric, humanized, fully human, and bispecific antibodies. In certain embodiments, antigen-binding fragments are produced, for example, by recombinant DNA techniques. In additional embodiments, antigen-binding fragments are produced by enzymatic or chemical cleavage of intact antibodies. Antigen-binding fragments include, but are not limited to, Fab, Fab′, F(ab)², F(ab′)₂, Fv, and single-chain antibodies. Examples of antibodies suitable for use in the invention include, without limitation, the antibodies listed in FIGS. 1A and 1B as well as Abagovomab, Abciximab, Actoxumab, Adalimumab, Afclimomab, Afutuzumab, Alacizumab, Alacizumab pegol, ALD518, Alemtuzumab, Alirocumab, Alemtuzumab, Altumomab, Amatuximab, Anatumomab mafenatox, Anrukinzumab, Apolizumab, Arcitumomab, Aselizumab, Altinumab, Atlizumab, Atorolimiumab, tocilizumab, Bapincuzumab, Basiliximab, Bavituximab, Bectumomab, Belimumab, Benralizumab, Bertilimumab, Besilesomab, Bevacizumab, Bezlotoxumab, Biciromab, Bivatuzumab, Bivatuzumab mertansine, Blinatumomab, Blosozumab, Brentuximab vedotin, Briakinumab, Brodalumab, Canakinumab, Cantuzumab mertansine, Cantuzumab mertansine, Caplacizumab, Capromab pendetide, Carlumab, Catumaxomab, CC49, Cedelizumab, Certolizumab pegol, Cetuximab, Citatuzumab bogatox, Cixutumumab, Clazakizumab, Clenoliximab, Clivatuzumab tetraxctan, Conatumumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Ecromeximab, Eculizumab, Edobacomab, Edrecolomab, Efalizumab, Efungumab, Elotuzumab, Elsilimomab, Enavatuzumab, Enlimomab pegol, Enokizumab, Enokizumab, Enoticumab, Enoticumab, Ensituximab, Epitumomab cituxetan, Epratuzumab, Erenumab, Erlizumab, Ertumaxomab, Etaracizumab, Etrolizumab, Evolocumab, Exbivirumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixckizumab, Keliximab, Labetuzumab, Lebrikizumab, Lemalesomab, Lerdelimumab, Lexatumumab, Libivirumab, Ligelizumab, Lintuzumab, Lirilumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxctumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxclumab, Ozanczumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pintumomab, Placulumab, Ponezumab, Prezalumab, Priliximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Sevirumab, Sibrotuzumab, Sifalimumab, Siltuximab, Simtuzumab, Siplizumab, Sirukumab, Solanczumab, Solitomab, Sonepcizumab, Sontuzumab, Stamulumab, Sulesomab, Suvizumab, Tabalumab, Tacatuzumab tetraxctan, Tadocizumab, Talizumab, Tanczumab, Taplitumomab paptox, Tefibazumab, Telimomab aritox, Tenatumomab, Tefibazumab, Telimomab aritox, Tenatumomab, Teneliximab, Teplizumab, Teprotumumab, Tezepclumab, TGN1412, Tremelimumab, Ticilimumab, Tildrakizumab, Tigatuzumab, TNX-650, Tocilizumab, Toralizumab, Tositumomab, Tralokinumab, Trastuzumab, TRBS07, Tregalizumab, Tremelimumab, Tucotuzumab celmoleukin, Tuvirumab, Ublituximab, Urclumab, Urtoxazumab, Ustekinumab, Vapaliximab, Vatelizumab, Vedolizumab, Veltuzumab, Vepalimomab, Vesencumab, Visilizumab, Volociximab, Vorsctuzumab mafodotin, Votumumab, Zalutumumab, Zanolimumab, Zatuximab, Ziralimumab and Zolimomab aritox.

The term “isolated antibody” as used herein refers to an antibody that has been identified and separated and/or recovered from a component of its natural environment. Contaminant components of its natural environment are materials which would interfere with diagnostic or therapeutic uses for the antibody, and may include enzymes, hormones, and other proteinaccous or nonproteinaccous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassic blue or, preferably, silver stain. Isolated antibody includes the antibody in situ within recombinant cells since at least one component of the antibody's natural environment will not be present. Ordinarily, however, isolated antibody will be prepared by at least one purification step.

The term “bind (ing)” of an antigen or other polypeptide includes, but is not limited to, the binding of a ligand polypeptide of the present invention to a receptor; the binding of a receptor polypeptide of the present invention to a ligand; the binding of an antibody of the present invention to an antigen or epitope; the binding of an antigen or epitope of the present invention to an antibody; the binding of an antibody of the present invention to an anti-idiotypic antibody; the binding of an anti-idiotypic antibody of the present invention to a ligand; the binding of an anti-idiotypic antibody of the present invention to a receptor; the binding of an anti-anti-idiotypic antibody of the present invention to a ligand, receptor or antibody, etc.

The term “immunoglobulin” refers to a protein consisting of one or more polypeptides substantially encoded by immunoglobulin genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions.

Full-length immunoglobulin “light chains” (about 25 Kd or about 214 amino acids) are encoded by a variable region gene at the NH₂-terminus (about 110 amino acids) and a kappa or lambda constant region gene at the COOH-terminus. Full-length immunoglobulin “heavy chains” (about 50 Kd or about 446 amino acids), are similarly encoded by a variable region gene (about 116 amino acids) and one of the other aforementioned constant region genes (about 330 amino acids). Heavy chains are classified as gamma, mu, alpha, delta, or epsilon, and define the antibody's isotype as IgG (such as IgG1, IgG2, IgG3 and IgG4), IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd Edition, Raven Press, NY (1989)), Chapter 7 (incorporated by reference in its entirety for all purposes).

An immunoglobulin light or heavy chain variable domain or region comprises “framework regions” (FRs) interrupted by “complementarity determining regions” (CDRs). Kabat et al. (1991), Sequences of Proteins of Immunological Interest, 5th Edition, Public Health Service, National Institutes of Health, Bethesda, Md. (1991); Chothia et al. (1987), J. Mol. Biol. 196:901-917 (both of which are incorporated herein by reference). FR residues are those variable domain residues other than CDR region residues as herein defined. The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen. Accordingly, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. Constant regions need not be present, but if they are, they must be substantially identical to human immunoglobulin constant regions—i.e., at least about 85-90%, preferably about 95% or more identical. Hence, all parts of a humanized immunoglobulin, except possibly the CDR's, are substantially identical to corresponding parts of natural human immunoglobulin sequences. Further, one or more residues in the human framework region may be back mutated to the parental sequence to retain optimal antigen-binding affinity and specificity. In this way, certain framework residues from the non-human parent antibody are retained in the humanized antibody in order to retain the binding properties of the parent antibody while minimizing its immunogenicity. The term “human framework region” as used herein includes regions with such back mutations. A “humanized antibody” is an antibody comprising a humanized light chain and a humanized heavy chain immunoglobulin. For example, a humanized antibody would not encompass a typical chimeric antibody as defined below, e.g., because the entire variable region of a chimeric antibody is non-human.

The monoclonal antibodies and antibody constructs of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al. (1984), Proc. Natl. Acad. Sci. USA, 81:6851-6855). Chimeric antibodies of interest herein include “primitized” antibodies comprising variable domain antigen-binding sequences derived from a non-human primate (e.g., Old World Monkey, Ape, etc.) and human constant region sequences. A variety of approaches for making chimeric antibodies have been described. See e.g., Morrison et al. (1985), Proc. Natl. Acad. Sci. U.S.A. 81:6851; Takeda et al. (1985), Nature 314:452, Cabilly et al., U.S. Pat. No. 4,816,567; Boss et al., U.S. Pat. No. 4,816,397; Tanaguchi et al., EP 0171496; EP 0173494; and GB 2177096.

The terms “human antibody” and “fully human antibody” each refer to an antibody that has an amino acid sequence of a human immunoglobulin, including antibodies isolated from human immunoglobulin libraries or from animals transgenic for one or more human immunoglobulins and that do not express endogenous immunoglobulins; for example, Xenomouse® antibodies and antibodies as described by Kucherlapati et al. in U.S. Pat. No. 5,939,598.

The term “genetically altered antibodies” means antibodies wherein the amino acid sequence has been varied from that of a native antibody. Because of the relevance of recombinant DNA techniques in the generation of antibodies, one need not be confined to the sequences of amino acids found in natural antibodies; antibodies can be redesigned to obtain desired characteristics. The possible variations are many and range from changes to just one or a few amino acids to complete redesign of, for example, the variable and/or constant region. Changes in the constant region will, in general, be made in order to improve or alter characteristics, such as complement fixation, interaction with membranes and other effector functions, as well as manufacturability and viscosity. Changes in the variable region will be made in order to improve the antigen binding characteristics.

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and one heavy chain that contains more of the constant region, between the C_{H1} and C_{H2} domains, such that an interchain disulfide bond can be formed between two heavy chains to form a F(ab′)₂ molecule.

A “F(ab′)₂ fragment” contains two light chains and two heavy chains containing a portion of the constant region between the C_{H1} and C_{H2} domains, such that an interchain disulfide bond is formed between two heavy chains.

The terms “Fv fragment” and “single chain antibody” refer to polypeptides containing antibody variable regions from both heavy and light chains but lacking constant regions. Like a whole antibody, it is able to bind selectively to a specific antigen. With a molecular weight of only about 25 kDa, Fv fragments are much smaller than common antibodies (150-160 kD) which are composed of two heavy protein chains and two light chains, and even smaller than Fab fragments (about 50 kDa, one light chain and half a heavy chain).

A “single domain antibody” is an antibody fragment consisting of a single domain Fv unit, e.g., V_{H} or V_{L}. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (.about.50 kDa, one light chain and half a heavy chain) and single-chain variable fragments (.about.25 kDa, two variable domains, one from a light and one from a heavy chain). The first single-domain antibodies were engineered from heavy-chain antibodies found in camelids. Although most research into single-domain antibodies is currently based on heavy chain variable domains, light chain variable domains and nanobodies derived from light chains have also been shown to bind specifically to target epitopes.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

In some embodiments, an antigen binding protein of the present invention selectively inhibits the human antigen of the antibody from which it is derived. For example, the antigen binding protein having the sequence of antibody AF as substituted as described herein will selectively inhibit the antigen in FIG. 1B of antibody AF. An antibody or functional fragment thereof “selectively inhibits” a specific receptor or ligand relative to other receptors or ligands when the IC50 of the antibody in an inhibition assay of the specific receptor is at least 50-fold lower than the IC50 in an inhibition assay of another “reference” ligand or receptor. An “IC50” is the dose/concentration required to achieve 50% inhibition of a biological or biochemical function. With radioactive ligands, IC50 is the concentration of a competing ligand that displaces 50% of the specific binding of the radioactive ligand. The IC50 of any particular substance or antagonist can be determined by constructing a dose-response curve and examining the effect of different concentrations of the drug or antagonist on reversing agonist activity in a particular functional assay. IC50 values can be calculated for a given antagonist or drug by determining the concentration needed to inhibit half of the maximum biological response of the agonist. Thus, the IC50 value for any anti-PCSK9 antibody or functional fragment thereof, for example, can be calculated by determining the concentration of the antibody or fragment needed to inhibit half of the maximum biological response of PCSK9 in activating the human PCSK9 receptor in any functional assay. An antibody or functional fragment thereof that selectively inhibits a specific ligand or receptor is understood to be a neutralizing antibody or neutralizing fragment with respect to that ligand or receptor. Thus, in some embodiments, the anti-PCSK9 antibody or functional fragment is a neutralizing antibody or fragment of human PCSK9.

The substituted antigen binding proteins of the present invention can cross-block the unsubstituted antibodies from which they are derived. The terms “cross-block,” “cross-blocked,” and “cross-blocking” are used interchangeably herein to mean the ability of an antigen binding protein to interfere with the binding of other antigen binding proteins (e.g., antibodies or binding fragments) to a target (e.g., human PCSK9). The extent to which an antibody or binding fragment is able to interfere with the binding of another to a target and therefore whether it can be said to cross-block, can be determined using competition binding assays. In some embodiments, a cross-blocking antigen binding protein of this invention reduces binding of a reference antibody to the target antigen between about 40% and 100%, such as about 60% and about 100%, specifically preferably between about 70% and 100%, and more specifically preferably between about 80% and 100%. A particularly suitable quantitative assay for detecting cross-blocking uses a Biacore machine which measures the extent of interactions using surface plasmon resonance technology. Another suitable quantitative cross-blocking assay uses a FACS-based approach to measure competition between antibodies in terms of their binding to the target antigen.

The term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., .alpha.-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids”, which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

An “isolated nucleic acid molecule” is a nucleic acid molecule that is not integrated in the genomic DNA of an organism. For example, a DNA molecule that encodes a heavy chain of an antibody that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGchec et al. (1993), Mol. Endocrinol., 7:551), cyclic AMP response elements (CREs), serum response elements (SREs; Treisman (1990), Seminars in Cancer Biol., 1:47), glucocorticoid response elements (GREs), and binding sites for other transcription factors, such as CRE/ATF (O'Reilly et al. (1992), J. Biol. Chem., 267:19938), AP2 (Ye et al. (1994), J. Biol. Chem., 269:25728), SPI, CAMP response element binding protein (CREB; Locken (1993), Gene Expr., 3:253) and octamer factors (see, in general, Watson et al. (1987), eds., Molecular Biology of the Gene, 4th Edition, The Benjamin/Cummings Publishing Company, Inc., and Lemaigre et al. (1994), Biochem. J., 303:1). If a promoter is an inducible promoter, then the rate of transcription increases in response to an inducing agent. In contrast, the rate of transcription is not regulated by an inducing agent if the promoter is a constitutive promoter. Repressible promoters are also known.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific”, “tissue-specific”, or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces an antagonist of the present invention from an expression vector. In contrast, such an antagonist can be produced by a cell that is a “natural source” of said antagonist, and that lacks an expression vector.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “fusion protein” is a hybrid protein expressed by a nucleic acid molecule comprising nucleotide sequences of at least two genes. For example, a fusion protein can comprise at least part of an antibody heavy chain fused with a polypeptide that binds an affinity matrix or another target of interest.

The term “receptor” denotes a cell-associated protein that binds to a bioactive molecule termed a “ligand.” This interaction mediates the effect of the ligand on the cell. Receptors can be membrane-bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor. In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increased cyclic AMP production, mobilization of cellular calcium, mobilization of membrane lipids, cell adhesion, hydrolysis of inositol lipids and hydrolysis of phospholipids.

The term “expression” refers to the biosynthesis of a gene product. For example, in the case of a structural gene, expression involves transcription of the structural gene into mRNA and the translation of mRNA into one or more polypeptides.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a polyhistidine tract, protein A (Nilsson et al. (1985), EMBO J. 4:1075; Nilsson et al. (1991), Methods Enzymol., 198:3), glutathione S transferase (Smith et al. (1988), Gene, 67:31), Glu-Glu affinity tag (Grussenmeyer et al (1985), Proc. Natl. Acad. Sci. USA 82:7952), substance P, FLAG® peptide (Hopp et al. (1988), Biotechnology 6:1204), streptavidin binding peptide, or other antigenic epitope or binding domain. See, in general, Ford et al. (1991), Protein Expression and Purification, 2:95. DNA molecules encoding affinity tags are available from commercial suppliers (e.g., Pharmacia Biotech, Piscataway, N.J.).

The terms “acidic residue” and “charged negative residue” refer to amino acid residues having sidechains comprising acidic groups. Exemplary acidic or charged negative residues include D and E.

The term “amide residue” refers to amino acids having sidechains comprising amide derivatives of acidic groups. Exemplary amide residues include N and Q.

The term “aromatic residue” refers to amino acid residues having sidechains comprising aromatic groups. Exemplary aromatic residues include F, Y, and W.

The terms “basic residue” and “charged positive residue” refer to amino acid residues having sidechains comprising basic groups. Exemplary basic or charged positive residues include H, K, and R.

The terms “hydrophilic residue” and “polar uncharged residue” refer to amino acid residues having sidechains comprising polar groups. Exemplary hydrophilic or polar uncharged residues include C, S, T, N, and Q.

The terms “non-functional residue” and “small hydrophobic residue” refer to amino acid residues having sidechains that lack acidic, basic, or aromatic groups. Exemplary non-functional, small hydrophobic residues include M, G, A, V, I, L and norleucine (Nle).

One aspect of this invention concerns PCSK9 binding polypeptides. “PCSK9-binding polypeptide” means a polypeptide that binds proprotein convertase subtilisin/kexin type 9 (PCSK9) protein. In some cases, the PCSK9-binding polypeptide blocks binding of PCSK9 to low-density lipid receptors (LDLRs). Such blocking PCSK9-binding polypeptides can be monoclonal antibodies (mAbs) and can be one of the following:
a. a mAb comprising a heavy chain polypeptide having an amino acid sequence of SEQ ID NO: 136 and a light chain polypeptide having an amino acid sequence of SEQ ID NO: 134 (antibody AK, evolocumab), or an antigen-binding fragment thereof;
b. a mAb that competes with evolocumab for binding to PCSK9;
c. a mAb comprising:
   i. a heavy chain polypeptide comprising the following complementarity determining regions (CDRs): a heavy chain CDR1 that is a CDR1 in SEQ ID NOs: 376 or 378; a heavy chain CDR2 that is a CDR2 in SEQ ID Nos: 376 or 378; a heavy chain CDR3 that is a CDR3 in SEQ ID NOs: 376 or 378, and
   ii. a light chain polypeptide comprising the following CDRs: a light chain CDR1 that is a CDR1 in SEQ ID NOs: 377 or 379; a light chain CDR2 that a CDR2 in SEQ ID NOs: 377 or 379; and a light chain CDR3 that is a CDR3 in SEQ ID NOs: 377 or 379;
d. a mAb that binds to at least one of the following residues of PCSK9, the PCSK9 comprising an amino acid sequence of SEQ ID NO: 369: S153, D188, I189, Q190, S191, D192, R194, E197, G198, R199, V200, D224, R237, and D238, K243, S373, D374, S376, T377, F379, I154, T1897, H193, E195, I196, M201, V202, C223, T228, S235, G236, A239, G244, M247, I369, S372, C375, C378, R237, and D238;
e. a mAb that binds to PCSK9 at an epitope on PCSK9 that overlaps with an epitope that is bound by an antibody that comprises:
   i. a heavy chain variable region of the amino acid sequence in SEQ ID NO: 136; and
   ii. a light chain variable region of the amino acid sequence in SEQ ID NO: 134, and
   iii. wherein the epitope of the mAb further overlaps with a site to which binds an epidermal growth factor-like repeat A (EGF-A) domain of the low density lipoprotein receptor (LDLR) protein (Horton, Cohen, & Hobbs (2007), Trends Biochem Sci, 32 (2), 71-77. doi: 10.1016/j.tibs.2006.12.008; Seidah & Prat (2007), J Mol Med (Berl), 85 (7), 685-696;
f. a mAb that comprises a heavy chain polypeptide comprising the following complementarity determining regions (CDRs):
   i. heavy chain CDR1, CDR2, and CDR3 having an amino acid sequence of SEQ ID NOs: 373, 374, and 375, respectively; and
   ii. light chain CDR1, CDR2, and CDR3 having an amino acid sequence of SEQ ID NOs: 369, 370, and 371, respectively; or
g. a mAb that comprises the heavy chain variant region sequence of SEQ ID NO: 378 and the light chain variant region sequence of SEQ ID NO: 379.

### PREFERRED EMBODIMENTS

### Correlation of Global Sequence Features to Viscosity

The main goal of the study reported in Example 1 of this specification was to identify a link between viscosity and amino acid sequence, or global sequence features of IgG monoclonal antibodies with the purpose of reducing viscosity of high-concentration monoclonal antibody formulations. For that, viscosity values of 43 different monoclonal antibodies were measured at 150 mg/ml to provide a wide range of values from 5 to 33 cP (FIGS. 1A and 1B). Main global sequence features of the monoclonal antibodies, such as light and heavy chain types, their subtypes (germlines), and pI were calculated and correlated to viscosity, but did not immediately reveal significant correlations (FIGS. 1A and 1B). Polymorphisms (known as allotypes) within the IgG isotypes were described using serological reagents derived from humans (Ropartz, C., Schanfield, M. S., and Steinberg, A. G. (1976), “Review of the notation for the allotypic and related markers of human immunoglobulins,” WHO meeting on human immunoglobulin allotypic markers, held 16-19 Jul. 1974, Rouen, France; report amended June 1976, J Immunogenet. 3, 357-362) and correlated to certain amino acid residues in several specific positions in conserved regions of heavy and light chains (Jefferis and Lefranc (2009) Human immunoglobulin allotypes: possible implications for immunogenicity, mAbs 1, 332-338.) (Vidarsson, G., Dekkers, G., and Rispens, T. (2014) IgG subclasses and allotypes: from structure to effector functions, Front Immunol. 5, 1-17). The allotypes introduce a few different residues (described below) in otherwise conserved regions of light and heavy chains. All kappa light chains used in this study were the same (3) allotype (featuring residues A153, V191 in EU numbering). All IgG2 heavy light chains were the same (n-) allotype (featuring P189). Four IgG1 heavy chain allotypes were described (including the following related residues in EU numbering): f (R214); z (K214); a (D356, L358) and x (G431) (Jefferis & Lefranc, 2009) (Vidarsson et al., 2014). IgG1 heavy chains with alternative residues in the positions E356, M358 and A431 do not constitute allotypes because these amino acid residues are present in other IgG subclasses. IgG1 allotype (x) was not present in the study; all IgG1 heavy chains had A431. IgG1 heavy chain allotypes (f), (z), (a) and related residues are shown in FIGS. 1A and 1B.

The antibodies in FIGS. 1A and 1B are sorted by viscosity. The table in FIGS. 1A and 1B includes the monoclonal antibody name, measured concentrations, measured viscosity values and global sequence parameters including type, subtype and calculated pI. IgG1 type, lambda light chains and VH1 heavy chain subtype are in boldface type.

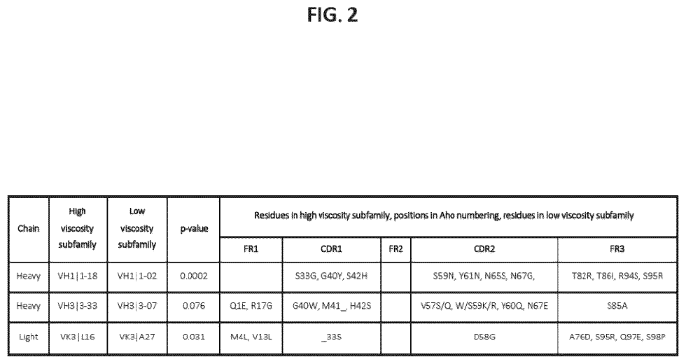

IgG1 and IgG2 heavy chains and kappa and lambda light chains were rather evenly distributed across the viscosity range. A sequence assessment of subtypes revealed several high viscosity and low viscosity subtype pairs: VH1|1-18 and VH1|1-02; VH3|3-33 and VH3|3-07; VK3|L16 and VK3|A27 with the probability of random correlation at 0.0002; 0.076 and 0.031, respectively, correlating to viscosity residues (FIG. 2). The study looked for viscosity correlations to D and J region sequences but found no significant correlation.

FIG. 2 shows p-values, which indicate probability of random correlation to viscosity. FIG. 2 also shows residues in high viscosity subtype, positions in Aho numbering, and residues in low viscosity subtypes.

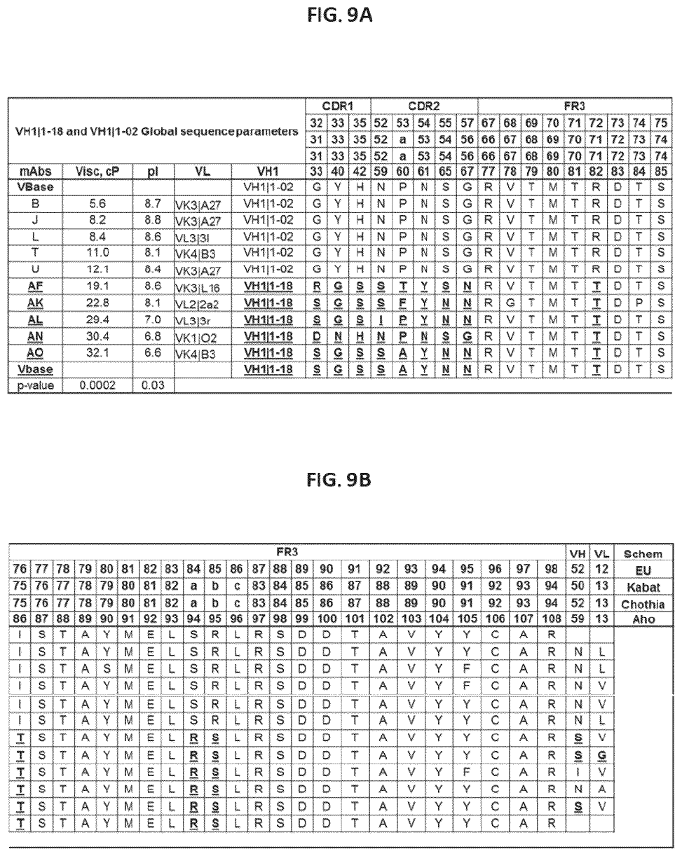

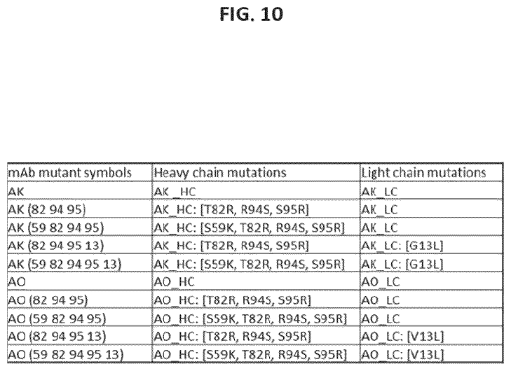

Among the fourteen IgG molecules of VH1 subtype, high viscosity was strongly associated with VH1 1-18 subtype and low viscosity with VH1 1-01 subtype with the very low probability of this being a random coincidence (FIG. 9).

In order to assess the correlation between the two subtypes and viscosity, probability of the same population mean by Student's t-test was calculated for VH1|1-02 versus VH1|1-18, VH3|3-33 versus VH3|3-07 and VK3|L16 versus VK3|A27 using t-test two-sample equal variants with a two-tailed distribution.

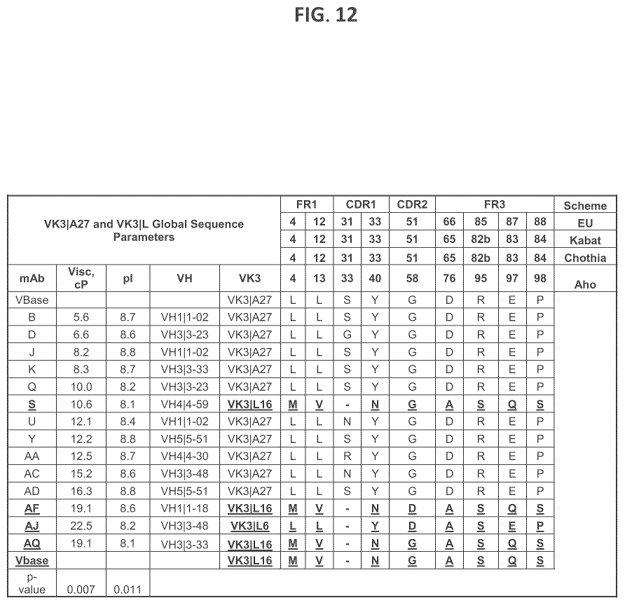

An association (t-test, p=0.031) of light chain VK3|L16 with high viscosity and VK3|A27 with low viscosity was detected (FIG. 2, FIG. 12, left side). VK3|L16 versus VK3|A27 and VH3|3-33 versus VH3|3-07 are discussed further in this specification. Due to the strong correlation to viscosity, VH1|1-18 and VH1|1-02 were further evaluated as follows. As the next step, 43 antibody chain sequences were aligned and assessed as follows.

### Sequence Alignment and Numbering System

Several IgG numbering systems exist, including:
EU—Edelman et al. (1969), “The covalent structure of an entire gamma immunoglobulin molecule,” Proc. Natl. Acad. Sci. U.S.A. 63, 78-85;
Kabat—Kabat et al. (1991), Sequences of proteins of immunological interest, Fifth Edition. NIH Publication No. 91-3242;
Chothia—Chothia et al. (1992), “Structural repertoire of the human VH segments,” J. Mol. Biol. 227:799-817; Tomlinson et al., (1995), “The structural repertoire of the human V kappa domain. EMBO J. 14:4628-4638;
Aho—Hoenegger et al. (2001), “Yet another numbering scheme for immunoglobulin variable domains: an automatic modeling and analysis tool,” J. Mol. Biol. 309, 657-670;
   and others. All four numbering systems mentioned above are illustrated in FIG. 9, right side for frame region 3 of the heavy chain of VH1 subtype. The Aho scheme was built by utilizing spatial positions of amino acid residues derived from more than 400 crystal structures of variable domains of different antibodies. The Aho numbering system defined by A. Honegger (citation above) was used in this work because it is a 3D structure-based numbering system. This creates an advantage specifically for residues in CDRs: Residues with the same numbers are located in similar spatial areas and are comparable across different IgG sequences. Since residue positions in the Aho numbering scheme are related to tertiary structure, they should be more associated with biophysical and biochemical properties and, possibly, viscosity. Aho numbering is aligned and correlated with the other main numbering schemes as it is shown in several tables of this specification. Any of the four numbering systems can be interchangeably used to identify the preferred amino acid substitutions. The heavy chain variable region ends at the following residues for different numbering systems: 149 Aho, 117 EU, 113 Kabat, 113 Chothia. The light chain variable region ends at 149 Aho, 107 EU, 107 Kabat, 107 Chothia. Aho numbering allocates more numbers for CDR regions instead of using letters for CDR residues as in Kabat and Chothia (for example 82b for Kabat and Chothia). As a result, Aho numbers for the same residues are often larger. Each variable region includes three complementarity determining regions (CDRs) and four framework regions (FRs) in the following sequence: FR1-CDR1-FR2-CDR2-FR3-CDR3-FR4. While CDRs provide great sequence diversity with the purpose of binding to antigens (CDR3 regions bind most often), FR sequences are more conserved and contain only a few differences, some of which are subtype specific.

### Correlation of Sequences to Viscosity

In addition to assessing the global sequence features, the variable regions' sequences were aligned to identify the residues responsible for the viscosity differences. In addition to visual observations, a software machine learning algorithm was developed and applied to identify the residues impacting viscosity the most and to predict antibodies' viscosity values from their sequences. The predictive model was constructed using charge and hydrophobicity of residues in Aho-aligned positions.

Heavy chain VH1 sequence alignment and assessment of the five sequences of high-viscosity VH1 1-18 subtype and five sequences of low-viscosity VH1 1-02 subtype molecules revealed that only 4 residues were different between the two subtypes in frames, all four located in framework 3 (FIG. 9, right side). A larger number of sequence differences were observed in CDRs, but they were left beyond the scope of the study, since CDRs are often involved in binding to the antigens, and engineering (mutating residues in) CDRs with a goal of reducing viscosity would carry a significant risk of losing potency. The subtype-related differences in viscosity and residues in FR3 indicated that the following amino acid substitutions could potentially reduce viscosity in Aho numbering: T82R, T86I, R94S and S95R. The software algorithm supported the four substitutions and also suggested that the following two substitutions correlated with reduction of viscosity: light chain G13V/L in FR1 and heavy chain S59R/K from the edge of CDR2, the latter correlation observed in VH3 subtype (FIG. 9, right side).

### In Silico Assessment of Suggested Amino Acid Substitutions

VH T82R. R82 occurs with high frequency in VH1-02. IgG structure modeling indicated that heavy chain Aho position 82 is a part of the upper core of the globulin fold and does not typically contact the antigen, but it can directly contact CDR backbones according to Ewert et al. (2003), “Biophysical properties of human antibody variable domains,” J. Mol. Biol. 325:531-553. HC82 has very conserved main chain-side chain H-bond interactions with CDR 1 and CDR2 backbone amides (Honegger et al., supra). R82 may also coordinate the backbone oxygen atoms of the CDR2 loop.

VH R94S and S95R. S94 and R95 occur with high frequency in VH1-02. These positions are located on the surface, away from the antigen binding domain and are considered a part of the lower core.

T86I. I86 occurs with high frequency in VH1-02. The data suggest substituting a hydrophobic residue (I) for hydrophilic one (T) on the surface, which can potentially lead to aggregation.

VH S59R/K—Within VH1 and VH3, a hydrophilic position 59 associates with lower viscosity. VH S59R/K position has high structural and sequence variability, is fairly solvent exposed, and is directly in between residues 58 and 60, which are part of the upper core and may affect binding. The structure will likely depend directly on the differences in residues 58 and 60 (especially 58 if it is buried). R/K59 has low frequency of occurrence (below 2%), and was not observed in VH1 according to the amino acid residue frequency analysis. All R/K59s are accommodated within the VH3 dataset except one (VH4).

VL G13V/L—This position is structurally buried and is a part of the lower core of the variable domain according to Ewert et al., supra. From this point of view, a G13V mutation to a more hydrophobic residue should make the core stronger.

To summarize, in silico sequence analysis indicated that the proposed mutations do not introduce any additional glycosylation sites or sites susceptible to rapid degradation under physiological or mildly acidic formulation conditions (NG, NS, NT, DG, DH). VH T82R, T86I, R94S and S95R mutations would provide a switch from subtype VH1-18 to VH1-02 in frame region 3, so they should not introduce any unusual or rare motifs. The addition of VH S59R and VL G13V mutations was suggested by the software from the VH3 subtype, outside of VH1. None of the mutation sites is positioned close to the binding regions, except for S59R, which is at the edge of HC CDR2 and, therefore, represents a mild risk of interfering with potency/binding. Arginine is a very low frequency residue in position 59 (R59), so prediction of its impact is difficult. T86I was identified as a high risk for aggregation and removed from the list of mutations.

Produced Mutants and their Expression, Potency, Chemical Modifications, Glycosylation and Viscosity.

Taking into account the above considerations, several mutants were produced for two IgG2 antibodies AK and AO of the high-viscosity VH1-18 subtype with the goal of reducing viscosity while maintaining potency (FIG. 9B). FIG. 9B includes monoclonal antibody mutant symbols and related mutations on the heavy and light chain.

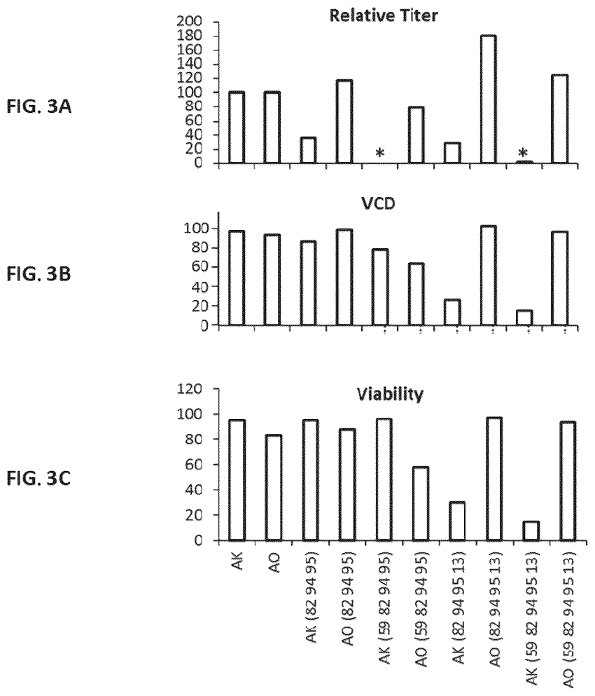

A very low expression level was observed for both AK mutants containing the S59R substitution (marked with * on FIG. 3). Viability and viable cell density was also low for one of them, AK (59 82 94 95 13). On the other hand, antibody AO mutants containing the S59R substitution produced a titer comparable to that of the AO parent molecule. Although the statistics were not sufficient to make a general conclusion about heavy chain position 59, the case suggested that a single amino acid substitution may dramatically alter expression. Chemical modifications, including oxidation, deamidation, isomerization and the glycosylation pattern, were similar among the two parents and their mutants as measured by peptide mapping LC-MS analysis.

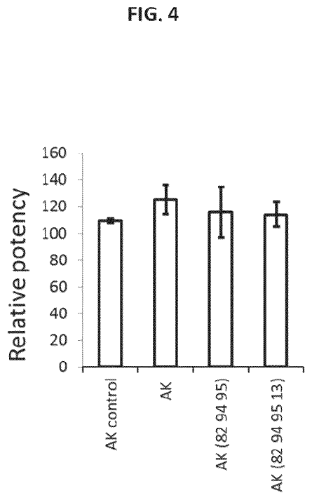

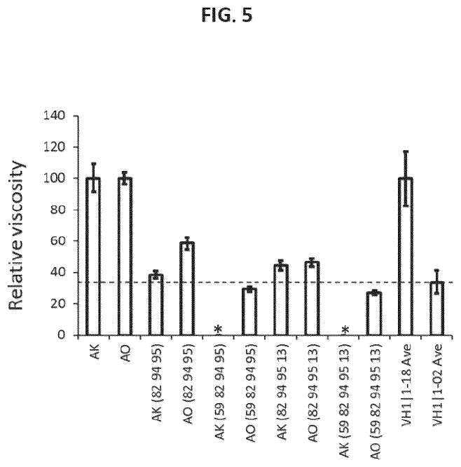

Potency values of the parent AK antibody and the two well-expressed mutants, measured through binding to PCSK9, were similar (FIG. 4). Finally, measured viscosity values of the AK and AO mutants were significantly lower than the parents, as predicted (FIG. 5). For example, the AK (82 94 95) mutant was at only 39% of the parent's viscosity. Viscosity of both AO mutants containing the S59R substitution was even lower, at approximately 28% of the parent's viscosity (FIG. 5). The S59R mutants did not express well for the AK antibody and the viscosity could not be measured. Average viscosity values for VH1|1-18 and VH1|1-02 germline subfamilies were added for comparison. A total of 12 consistent sequence differences were identified between VH1|1-18 and VH1|1-02 subfamilies, including 8 in CDRs and 4 in frame regions (FIG. 9). Three sites, all in frame region 3, were selected for amino acid substitutions from high viscosity VH1|1-18 to low viscosity VH1|1-02. The three point mutations in frames were introduced in two mAbs of VH1|1-18 subfamily (AK and AO) to convert only these residues to the residues present in VH1|1-02. Although chances to achieve the possible 2-fold decrease in viscosity were theoretically low ( 3/12), these substitutions rather unexpectedly produced desirable outcome: with only three substitutions the viscosity decreased approximately two-fold in both antibody molecules.

### Viscosity Versus pI

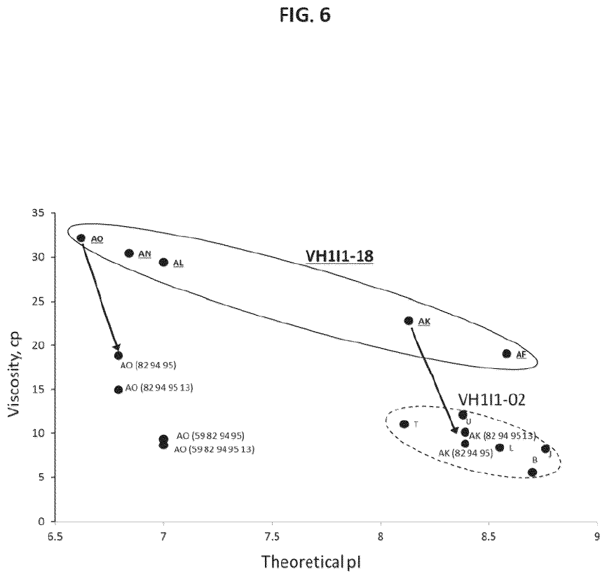

Although the dependence of viscosity from pI was not clear from the whole set of the 43 mAbs, the VH1 subset clearly showed that viscosity steadily increased when pI values of the mAbs decrease from pI 8.5 to pI 6.5 in the pH 5.2 formulation (FIG. 6). The shift between the high-viscosity VH1|1-18 mAbs and low-viscosity VH1|1-02 mAbs can be also seen. As predicted, T82R, R94S and S95R mutants of AK and AO antibodies moved approximately two fold down along the viscosity scale from the VH1|1-18 to the VH1|1-02 area on the plot (FIG. 6). Mutants AO (59 82 94 95) and AO (59 82 94 95 13) moved to even lower viscosity and to slightly higher pI, indicating that the S59R substitution, adopted from outside of the VH1 group, was effective in further reducing viscosity. Unfortunately, AK mutants containing R59 were not expressed well, suggesting that appearance of the low-frequency arginine residue in position 59 may affect expression.

An increase in pI for antibodies in formulations with pH<pI (for example, the mildly acidic formulation used in this study) typically leads to a decrease in viscosity. This result can be explained by the columbic repulsion of the positively charged antibody molecules. It is known that proteins, including antibodies, show poor solubility and high precipitation, which affect viscosity, at high concentrations. It is interesting that the VH1|1-18 to VH1|1-02 substitutions in frame region 3 resulted in a two-fold decrease in viscosity and only a minor increase in antibody pI values, suggesting that not the charge increase, but rather some structural changes may be responsible for the dramatic decrease in viscosity.

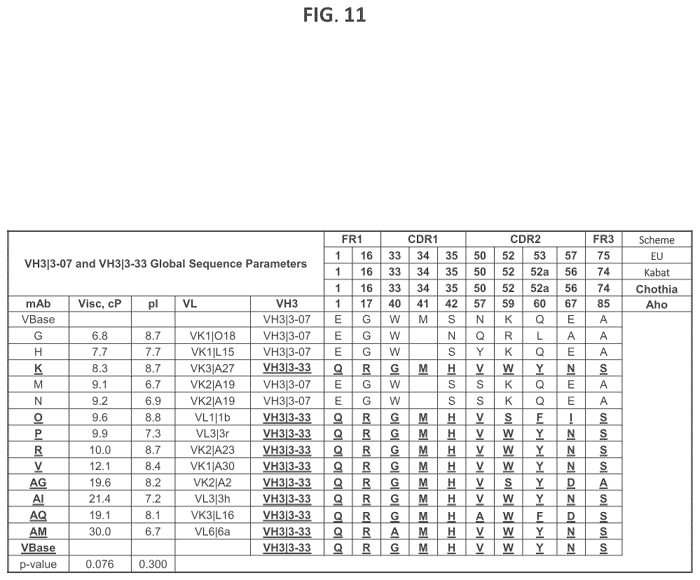

After superimposing crystallography structures of hundreds of Fab domains, hydrogen bond interactions for every VH and VL position were identified (Honegger et al. (2001), “Yet another numbering scheme for immunoglobulin variable domains: an automatic modeling and analysis tool,” J. Mol. Biol. 309:657-670). The data indicate that the identified positions may be bound to other residues through main-chain and side-chain hydrogen bond interactions (for Type III AK and AO antibody molecules). For example, 94 was bound to 77 in some VH Type III immunoglobulin structures; 95 to 18; 59 to 67, 66, 65, 61, 60 and VL13 to 146, 148. Hence, residue substitutions at these positions may change the interactions and the immunoglobulin fold. The crystal structure of AK antibody (Jackson et al., 2007), “The Crystal Structure of PCSK9: a Regulator of Plasma LDL-Cholesterol,” Structure 15:545-52) suggests that all three positions 82, 94 and 95 are on the very periphery of the Fab regions and exposed to solvent and other antibody molecules. Changes in FR3 positions in VH3 (FIG. 11) and VK3 (FIG. 12) also correlate to viscosity. One explanation of their role in viscosity is that these positions in FR3 are on the periphery of the molecule and are actively engaged in the intermolecular interactions during shear stress associated with motion through the injection needles and viscosity measurements.

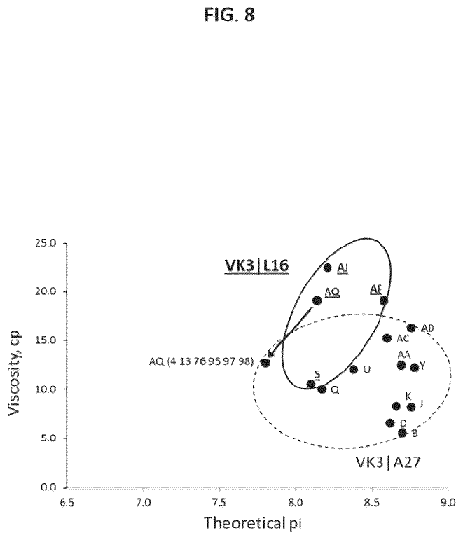

Lower viscosity VK3|A27 and higher viscosity VK3|L16 antibodies showed inverse correlation between viscosity and pI (FIG. 8, FIG. 12).

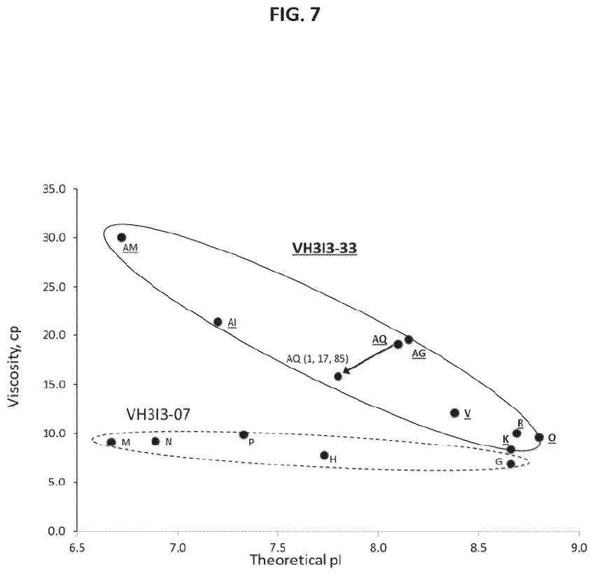

It needs to be mentioned that pI was a not very strong but in some cases useful predictor of viscosity. In general, viscosity decreased with increasing pI, and pI should be taken into account. For example, viscosity values of VH3|3-33 become lower and similar to VH3|3-07 for higher pI antibody molecules (FIG. 7). Hence, pI should be taken into account when predicting viscosity of VH3|3-33.

### Proposed Viscosity Reduction for VH3 and VK3 Germline Families

High-viscosity VH3|3-33 and low-viscosity VH3|3-07 antibodies on average have a large difference in viscosity, while occupying a similar pI range (FIG. 7). Unexpectedly, on average, VH3|3-07 had lower viscosity and also lower pI, which contradicts typical behavior reported in the literature. Viscosity values of the following VH3|3-33 molecules can be reduced by the mutations: AQ, AM, AI, AG.

Monoclonal antibodies with high-viscosity VK3|L16 and low-viscosity VK3|A27 light chains also showed a large viscosity difference while showing relatively small difference in pI values, again suggesting a structural difference between subfamilies (FIG. 8). Viscosity values of the following VK3|L16 molecule can be reduced by the mutations described in FIG. 11: antibodies AQ and AF.

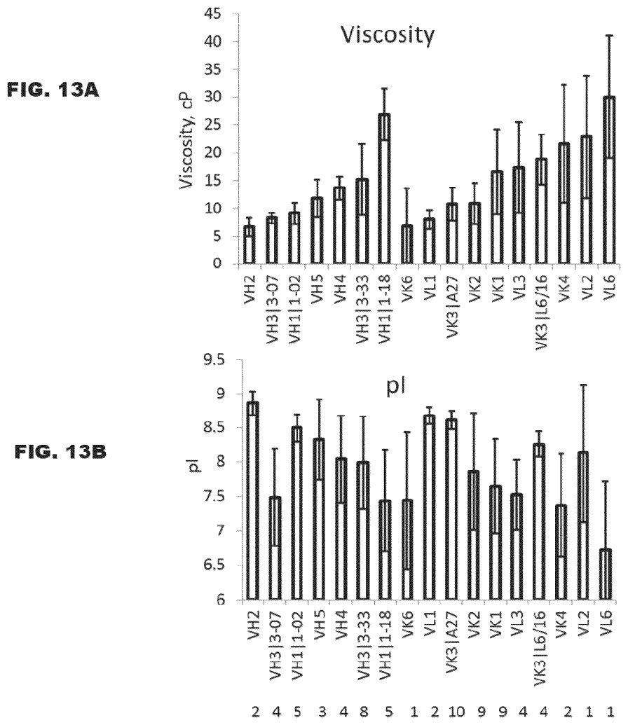

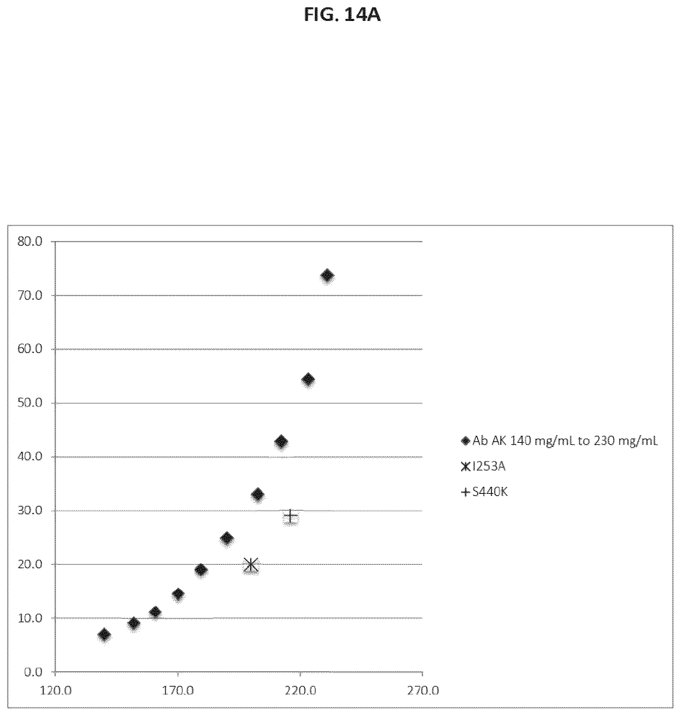

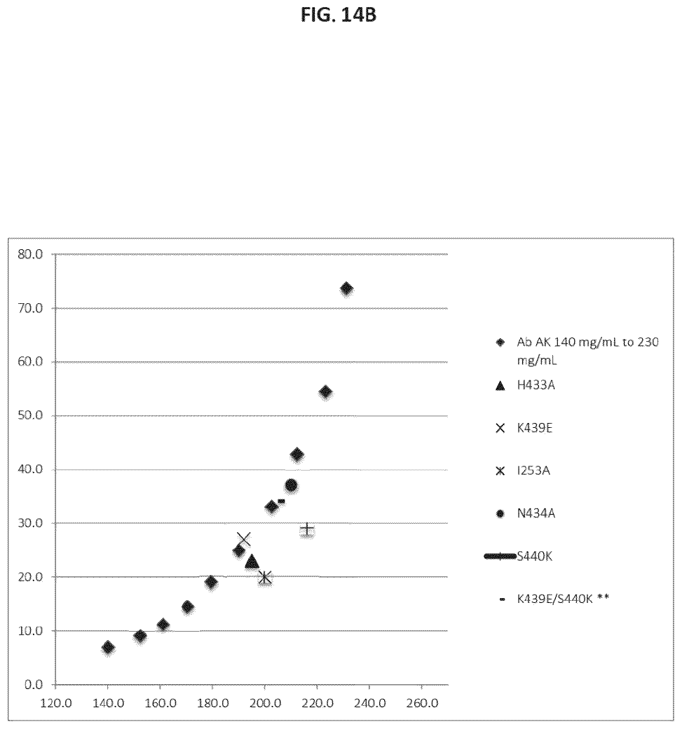

The analysis of global sequence features revealed the following high/low viscosity pairs: VH1|1-18/VH1|1-02; VK3|L16/VK3|A27; and VH3|3-33/VH3|3-07 with p-values of the correlation at 0.0002, 0.031 and 0.076, respectively (FIG. 2). Sequence positions and residues correlating to the viscosity differences were identified, and can be considered as candidates for viscosity lowering point mutations (FIG. 2). The performed above correlation of VH and VL families to viscosity indicates that theoretically antibodies with the following VH and VL combinations should have the lowest viscosity (FIGS. 13A and B): VH2, VH3|3-07, VH1|1-02 for VH and VK3, VL1, VK3|A27 for VL. Three antibodies of VH1|1-02 and VK3|A27 configuration practically occurred in our set and they indeed exhibited low viscosity values: B (5.6 cP), J (8.2 cP), Y (12.1 cP).

### Chemical Cross-linking Studies

A broad survey of the potential protein-protein interactions in viscous antibody solutions was attempted using chemical cross-linking at high protein concentrations. Chemical cross-linking is a classic biochemical technique used to demonstrate that specific portions of proteins interact with each other. In this paper we describe the use of a zero length chemical cross-linking reagent to identify potential protein-protein interactions in high concentration viscous antibody solutions. The chemical cross-linking results of viscous and non-viscous antibodies are used to construct a model of potential protein-protein interactions in solution.

Chemical cross-linking with EDC reveals the presence of a common chemically cross-linkable oligomeric distribution. The chemical cross-link that results in the oligomeric pattern is surprisingly not inter-molecular but rather an intra-molecular cross-link. The intra-molecular cross-link between the top of the Fc and the bottom of a Fab results in an antibody conformation that may favor the formation of Fc-Fc mediated antibody oligomers. The 1HZH antibody crystal structure contains an antibody hexamer in the asymmetric unit that has one Fab arm pinned against the Fc domain to facilitate the Fc-Fc interaction critical to form the IgG1 hexamer. The appearance of this conformation may or may not be critical to the formation of the hexamer in the protein crystal. The increased propensity to form the hexamer in solution when the Fab-Fc intra-molecular chemical cross-link is present suggests that the Fab arm pinned to the Fc may have increased propensity to form Fc-Fc based antibody oligomers.

### Decreasing Fc-Fc Interactions can Decrease Solution Viscosity

The scientific literature includes research on hexamer formation of antibodies related to interaction with Clq1 and CDC activity. Diebolder et al. found that anti-CD20 antibodies with certain Fc point mutations, including K439E and S440K, abrogated CDC activity but that the associated K439E/S440K double mutant restored CDC activity. Also I253A mutation decreased CDC activity. Diebolder et al. (2014), “Complement is Activated by IgG Hexamers Assembled at the Cell Surface,” Science 343:1260-3. Diebolder et al. did not associate the mutants they disclose with an effect on antibody viscosity. Similarly, van den Bremer et al (2015) found that charged residues at the C-terminus of an antibody could decrease Clq1 interaction due to a decreased ability to form IgG hexamer structures. The authors did not associate the presence of charged residues at the C-terminus of IgGs to an effect on antibody solution viscosity.

The observation of a hexamer of antibodies in viscous antibody solutions suggested that the Fc-Fc interactions that are present in the antibody hexamer crystal structure as well as the structure thought to form prior to the recruitment of Clq1 (complement) are likely present in viscous antibody solutions in the absence of the chemical cross-linker EDC. In order to test whether the Fc-Fc interaction might contribute to solution viscosity, Fc mutants based on the work done by Diebolder et al. were generated at Amgen. The materials were then evaluated in anti-PCSK9 formulation buffer by cone and plate rheology. The comparison of parent anti-PCSK9 antibody AK and Fc mutant anti-PCSK9 antibodies demonstrated that decreasing the affinity of Fc for Fc does decrease the solution viscosity of the antibody. The point mutants retained FcRn binding capacity and there were no changes to bioactivity. The ability of a double mutant which restored wild-type complement activity to return to wild type viscosity demonstrates that the decrease in viscosity can be reversed if the ability of Fc-Fc interactions is restored to wild-type levels. Taken together with the observation of Fc-Fc mediated oligomeric species increasing solution viscosity, there is a common Fc-mediated protein-protein interaction that contributes to antibody solution viscosity. It needs to be noted that out of five mutations, S440K, I253A, K439E, H433A, N434A identified by Diebolder et al. as reducing CDC activity and tested for viscosity in this work, only the first two showed reduced viscosity in high concentration anti-PCSK9 formulation, while K439E, H433A and N343A did not reduce viscosity in high concentration anti-PCSK9 formulation, indicating that direct correlation does not exist and that people skilled in the art could not correctly anticipate lower viscosity from the information provided by Diebolder et al. The K439E mutant was also evaluated in a high protein concentration sucrose formulation and found to be less viscous than the parent anti-PCSK9 mutant at the same concentration. The presence of Arginine in the anti-PCSK9 formulation may have contributed to charge screening that may have decreased the effectiveness of the negative charge introduced in the K439E mutant to decrease Fc-Fc interactions. The H433A and the N434A mutants have no obvious charge screening sensitivity like the K439E mutant.

The Fc-Fc interaction may influence solution viscosity by increasing the number of potential interactions possible in two potential ways. It could increase the number of interactions per antibody from two CDR mediated interactions to two CDR mediated interactions plus 2 Fc mediated interactions per antibody or it could change the number of free CDR ends available in oligomers present in solution. Given the fact that the Fc domain of IgG1, IgG2, IgG3 and IgG4 antibodies are highly similar, it is likely that the Fc-Fc mediated interactions are present in all antibodies. The analysis of non-viscous antibodies shows that the intra-molecular cross-link which indicates the presence of an Fc-Fc interaction is absent in contrast to a viscous antibody at the same concentration. This suggests that the Fc-Fc interaction, although theoretically possible, is absent in non-viscous antibodies. The CDR next nearest neighbor interaction may influence the relative distance between Fc's as well as the relative orientation to enhance Fc-Fc interaction. This may explain why viscous antibodies have an Fc-Fc interaction while non-viscous antibodies do not at room temperature.

The presence of an Fc-Fc interaction also increases the likelihood that an oligomer of antibodies (dimer, trimer, tetramer, etc.) contains the maximum number of free CDR ends. The larger number of free CDR ends increases the number of CDR next nearest neighbor interactions. This in turn may increase network formation propensity and increase solution viscosity as a result of more efficient “percolation.”

### C-Terminal Modifications to Reduce Viscosity

Certain modifications at the antibody C-terminus interfere with C1q binding and complement-dependent cytotoxicity (CDC). Van den Bremer et al. (2015), “Human IgG is produced in a pro-form that requires clipping of C-terminal lysines for maximal complement activation,” mAbs 7 (4): 672-80. The authors found that C-terminal lysine and C-terminal glutamic acid likely decreases the propensity of the Fc-Fc interactions that lead to a hexamer of antibodies that can most efficiently interact with C1q1. The authors constructed mutants of a CD20 antibody and a CD38 antibody having PGKP, PGKKP, PGKKKP, and PGE at the C-terminus. They found that these mutants showed significantly reduced or completely lost CDC activity. Thus, one may conclude that the mutations blocked the hexamerization previously correlated by the authors with CDC activity. Given the present correlation of hexamerization with viscosity, such mutations should also reduce viscosity of antigen binding proteins. It is thus reasonable to conclude that placement of positively charged or negatively charged amino acids at the C-terminus, whether placed there by addition or substitution of existing C-terminal amino acids, will reduce the viscosity of an antigen binding protein.

### Sequence Modification to Improve Pharmacokinetic Parameters

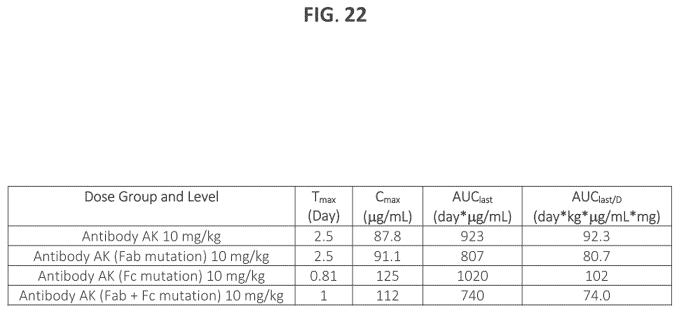

This invention also includes the discovery of improved pharmacokinetic properties in antigen binding proteins having mutations that may also reduce viscosity. In particular, S440K mutations have been found to improve both Tmax (the time after dosing at which the maximum concentration was observed) and Cmax (the maximum observed concentration measured after dosing). Mutants of antibody AK having S440K, optionally with other mutations, have been found to have Tmax reduced by more than half that of the parental antibody AK after subcutaneous injection of the mutants and the parental antibody at the same concentration. Such mutants have also been found to have Cmax that is 28% or 42% higher after subcutaneous injection of the mutants and the parental antibody. See FIG. 22.

### Nucleic Acids, Vectors, Host Cells

The invention also includes isolated nucleic acids encoding the bispecific antibodies of the invention, which includes, for instance, the light chain, light chain variable region, light chain constant region, heavy chain, heavy chain variable region, heavy chain constant region, linkers, and any and all components and combinations thereof of the bispecific antibodies disclosed herein. Nucleic acids of the invention include nucleic acids having at least 80%, more preferably at least about 90%, more preferably at least about 95%, and most preferably at least about 98% homology to nucleic acids of the invention. The terms “percent similarity”, “percent identity” and “percent homology” when referring to a particular sequence are used as set forth in the University of Wisconsin GCG® software program. Nucleic acids of the invention also include complementary nucleic acids. In some instances, the sequences will be fully complementary (no mismatches) when aligned. In other instances, there may be up to about a 20% mismatch in the sequences. In some embodiments of the invention are provided nucleic acids encoding both a heavy chain and a light chain of an antibody of the invention.

Nucleic acids of the invention can be cloned into a vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences, for example, an internal ribosomal entry site. The expression vector can be introduced into a cell by transfection, for example.

In another embodiment, the present invention provides an expression vector comprising the following operably linked elements; a transcription promoter; a first nucleic acid molecule encoding the heavy chain of a bispecific antigen binding protein, antibody or antigen-binding fragment of the invention; a second nucleic acid molecule encoding the light chain of a bispecific antigen binding protein, antibody or antigen-binding fragment of the invention; and a transcription terminator. In another embodiment, the present invention provides an expression vector comprising the following operably linked elements; a first transcription promoter; a first nucleic acid molecule encoding the heavy chain of a bispecific antigen binding protein, antibody or antigen-binding fragment of the invention; a first transcription terminator; a second transcription promoter a second nucleic acid molecule encoding the light chain of a bispecific antigen binding protein, antibody or antigen-binding fragment of the invention; and a second transcription terminator.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Recombinant host cells comprising such vectors and expressing the heavy and light chains are also provided.

### Purification

Methods of antibody purification are known in the art and can be employed with production of the antibodies and bispecific antibodies of the present invention. In some embodiments of the invention, methods for antibody purification include filtration, affinity column chromatography, cation exchange chromatography, anion exchange chromatography, and concentration. The filtration step preferably comprises ultrafiltration, and more preferably ultrafiltration and diafiltration. Filtration is preferably performed at least about 5-50 times, more preferably 10 to 30 times, and most preferably 14 to 27 times. Affinity column chromatography, may be performed using, for example, PROSEP® Affinity Chromatography (Millipore, Billerica, Mass.). In a preferred embodiment, the affinity chromatography step comprises PROSEP®-vA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The antibody product is preferably nanofiltered, for example, using a Pall DV 20 Nanofilter. The antibody product may be concentrated, for example, using ultrafiltration and diafiltration. The method may further comprise a step of size exclusion chromatography to remove aggregates. Further parameters of purification appear in the working examples hereinafter.

The bispecific antibodies, antibodies or antigen-binding fragments may also be produced by other methods known in the art, for example by chemical coupling of antibodies and antibody fragments.

Each manuscript, research paper, review article, abstract, patent application, patent or other publication cited in this specification is hereby incorporated by reference in its entirety.

### WORKING EXAMPLES

The invention is further illuminated by the following working examples, which exemplify but do not limit the scope of the invention

### Example 1

### Fab Mutations

### Materials

A set of 43 human and humanized recombinant monoclonal antibody molecules with different targets and different sequences were produced and purified according to a standard procedure (FIGS. 1A and 1B). The set with equivalent purity of >98% by size exclusion chromatography (SEC) was collected. Samples were concentrated in a 3 mL maximum volume using Amicon Ultrafiltration Stirred Cell Model 8003 (Millipore, Billerica, MA) at 2-8° C. at a maximum pressure of 30±10 psi. They were concentrated up to 150 mg/mL according to approximate volume depletion in a formulation buffer containing 20 mM acetate, 9% sucrose at pH 5.2 (without polysorbate), and final concentrations were determined (±10%) using the protein's absorbance at 280 nm (after dilution to end up within 0.1-1 absorbance units (AU)) and a protein-specific extinction coefficient.

Several low viscosity mutants of two mAbs were produced, purified and formulated according to a similar standard procedure. They included an antibody against proprotein convertase subtilisin/kexin type 9 (PCSK9, antibody AK) and against macrophage colony-stimulating factor (M-CSF, AO).

### Viscosity Measurements

Viscosity analysis was performed on a Brookfield LV-DVIII cone and plate instrument (Brookfield Engineering, Middleboro, MA, USA) using a CP-40 spindle and sample cup. All measurements were performed at 25° C., controlled by a water bath attached to the sample cup. Multiple viscosity measurements were collected, manually within a defined torque range (10-90%) by increasing the RPM of the spindle. Measurements were averaged in order to report one viscosity value per sample to simplify the resulting comparison chart

### Sequence Alignment

A structure-based sequence alignment was performed by an Ab Initio software tool developed using Excel Macros downloaded from the Department of Biochemistry of Zürich University.

### Example 2

### Fc Mutations

### Expression and Purification of Mutants

### Materials and Methods

Anti-C-kit antibody (antibody BA, SEQ ID NOS: 174 and 176, encoded by nucleic acids of SEQ ID NOS: 173 and 175, respectively), anti-sclerostin antibody AH, and anti-PCSK9 antibody AK
Anti-streptavidin IgG1 and IgG2.
1-ethyl-3-[3-dimethylaminopropyl] carbodiimide hydrochloride (EDC)
Solubilization with n-methyl-2-pyrrolidone
Size Exclusion High Performance Liquid Chromatography (SE-HPLC) with Light Scattering (LS)
Reduced and alkylated reversed phase High Performance Liquid Chromatography (RA RP-HPLC)
Trypsin peptide map with electro spray ionization mass spectrometry (ESI-MS)
Cone and plate viscometer

### Results

### SE-HPLC of EDC Cross-Linking of High Concentration Monoclonal Antibody Solutions Reveals Propensity to Form Oligomers

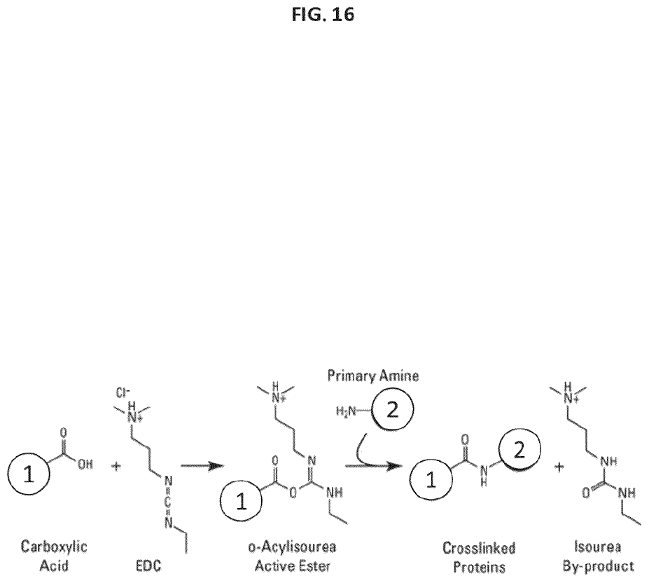

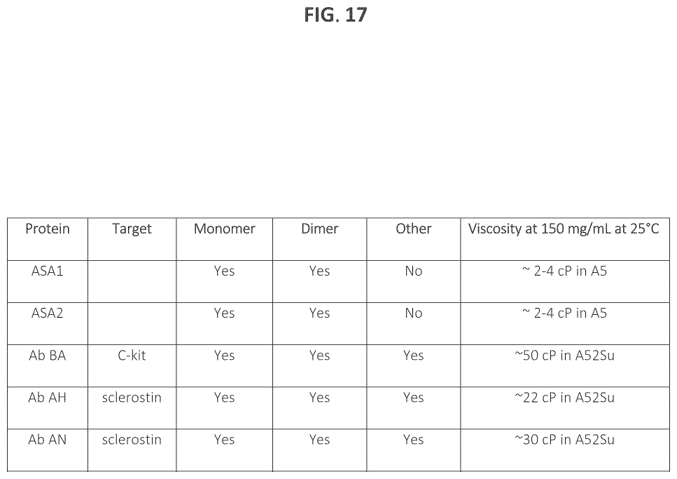

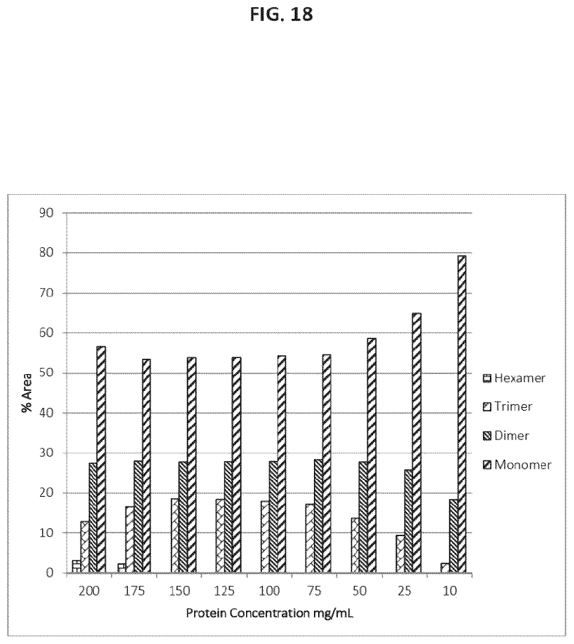

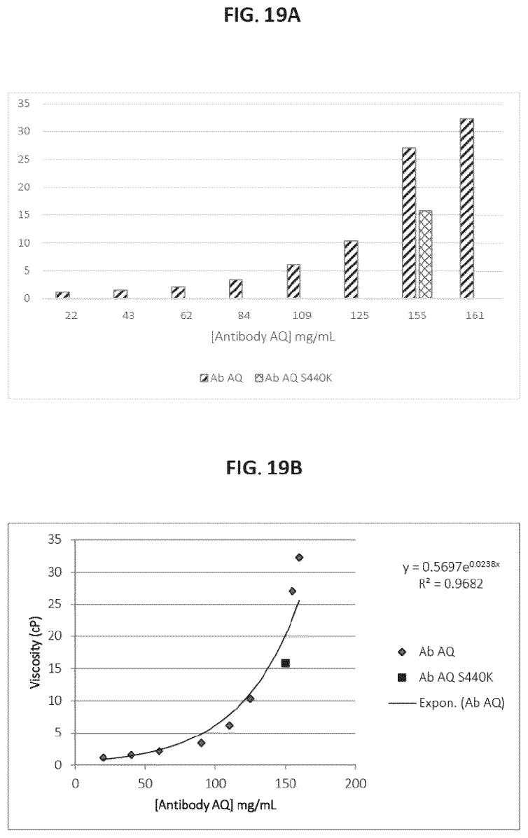

The compound 1-ethyl-3-[3-dimethylaminopropyl] carbodiimide hydrochloride (EDC) was used to chemically cross-link acidic residues in the antibody to primary amines in the antibody (N-terminus and/or Lys residues) and has been used in other studies to determine the regions of protein-protein interaction. The proximity of the carboxyl group to the primary amine is critical as an amide bond is formed between the two groups (FIG. 16). The cross-links that form are likely salt bridges that are present in solution. Carraway and Koshland, Jr. (1972). “Carbodiimide modification of proteins.” Methods Enzymol 25:616-623. A panel of antibodies was chemically cross-linked with EDC under identical solution conditions. Previous rheological studies had determined that some of the antibodies in the panel were viscous and some were not (FIG. 17). The non-viscous antibodies had small increases in dimer content but did not contain large amounts of higher order. In contrast, the viscous antibodies contained large amounts of dimer as well as higher order oligomers. A summary is provided in FIG. 17. All of the antibodies that were identified as viscous contained EDC cross-linked species that appeared to be larger than dimer. The appearance of the larger oligomeric species is concentration dependent (antibody AH is shown in FIG. 18 as an example). In order to facilitate further analysis, chemical cross-linking conditions were changed to drive the cross-linking reaction to completion. The solutions became solids after chemical cross-linking at 200 mg/mL. The solids were re-solubilized with buffer or a buffered 3% NMP solution. SE-HPLC analysis of both samples showed that the samples were similar. The buffered 3% NMP solution solubilized the protein significantly faster with more of the material going into solution. Antibody AH was re-solubilized and analyzed further as an example.

Size Analysis by SE-HPLC with Online LS

The cross-linked antibody solutions were analyzed by SE-HPLC with online light scattering to determine the size of the eluting species. SE-HPLC was conducted with online light scattering analysis of antibody AH after EDC chemical cross-linking and resolublization with 3% NMP. SE-HPLC revealed three peaks present in the UV and RI. The first peak was identified as a species with a mass of 840.5 kD. This is close to the expected mass for a hexamer of antibody AH of 873.2 kD. Another species had a mass of 494.6 kD, which is close to the predicted mass of 436.6 kD for a trimer of antibody AH. The third species showed a mass of 139.9 kD, which is close to the predicted mass of 145.5 kD for a monomer of antibody AH.

### Reduced and Alkylated Reversed Phase HPLC of EDC Cross-Linked Antibody AH

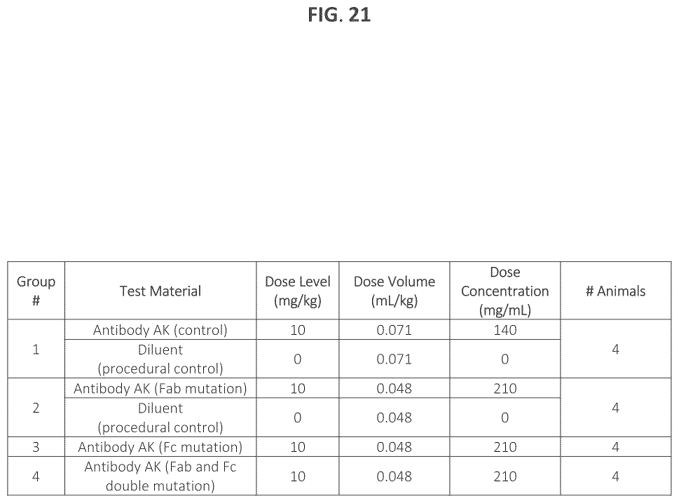

The cross-linked antibody solutions were analyzed by reduced and alkylated reversed phase high performance liquid chromatography. The recovery of the majority of both light chain (LC) and heavy chain (HC) was unexpected as it was presumed that either the HC or the LC would be cross-linked to one another to form non-native LC-HC peptides or non-native LC-LC or HC-HC peptides reflecting the cross-linked oligomers observed by SEC analysis. In the case of antibody AH, the LC eluted in the same place with the exact same mass as non-cross-linked antibody AH LC. There were changes in the HC in antibody AH that are concentration dependent. There is a small amount of HC-HC cross-linked material (eluting at about 33 minutes, FIG. 21) that had an identified mass of 100 kD. At 150 mg/mL and 200 mg/mL, the distribution of HC species is similar. At lower protein concentrations, the distribution includes more of a species that elutes at about 28.5 min. The distribution of HC species correlates with the amount of oligomer present in each sample as analyzed by SEC with the 200 mg/mL sample containing the largest amount of hexamers and the 10 mg/ml sample containing very little oligomer. The pattern was observed in other viscous antibody solutions.

### Example 3

### Nonhuman Primate Study of Pharmacokinetics and Pharmacodynamics (PKPD) of the Anti-PCSK9 Parent Antibody AK and Low Viscosity Mutants

Materials: antibody AK and its mutants. All mutations in heavy chain.
Fab Mutant: T82(72)R, R94(84)S, S95(85)R; Aho numbering (actual numbering)
Fc Mutant S(434)K (S440K in EU numbering):
Double Mutant: T82(72)R, R94(84)S, S95(85)R; S(434)K:

Four groups of 4 male cynomolgus monkeys were used in this study. Each group received 1 subcutaneous (SC) dose of 10 mg/kg as follows. Group 1 received parent antibody AK (140 mg/ml); Group 2 received Fab mutant (210 mg/ml); Group 3 received Fc mutant (210 mg/ml); Group 4: Fab/Fc mutant (210 mg/ml). Groups 1 and 2 also had diluent control SC dose. Fab mutant included the following substitutions in positions T82(72)R, R94(84)S, S95(85)R. Fc mutant included substitution in position S(434)K.

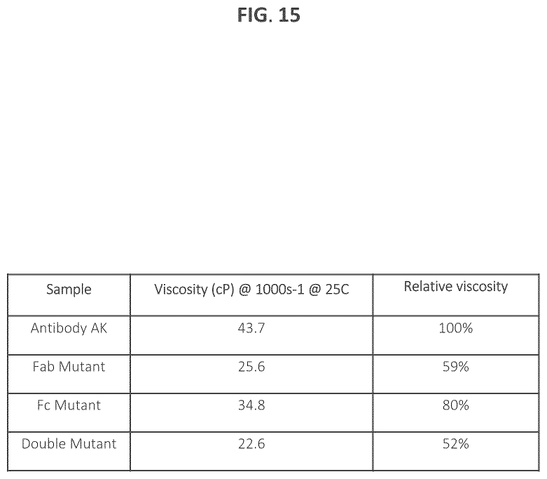

To measure viscosity at 210 mg/ml, the parent and all mutants were formulated at 210 mg/mL in 10 mM Acetate, 155 mM N-acetyl arginine (NAR), 70 mM ArgHCl, pH 5.4, 0.01% Polysorbate 80. Viscosity was measured using ARG2 cone/plate at 1000 sec-1 and 25 C. See FIG. 15.

### Study Design Outline:

4 groups of 4 male cynomolgus monkeys.
   Each group received 1 SC dose (10 mg/kg) of:
Group 1: parent antibody AK (140 mg/ml)
Group 2: Fab mutant (210 mg/ml)
Group 3: Fc mutant (210 mg/ml)
Group 4: Fab/Fc mutant (210 mg/ml)
   Groups 1 and 2 also had diluent control subcutaneous dose.
   Skin biopsies taken at injection site 3 days after dosing.
   Histopath analysis performed
   Plasma LDL, HDL, total cholesterol and PK followed for 6 weeks post-dose

### Study Conclusions

All 4 homologues produced marked LDL lowering
Maximal reduction 2 weeks after dosing: 1 week later than previously observed for parent
The nadir of the effect was not quite as profound for the Fab/Fc mutant (˜78% vs ˜90%)
Return to baseline appears slightly more accelerated for the Fc mutant
PK: Mean exposures were similar (based on Cmax and AUClast) between all treatment groups (within 1.4-fold)
Fab and/or Fc mutations in anti-PCSK9 antibody AK had no significant effect on injection site reactions (ISR) or Pharmacokinetics and Pharmacodynamics (PKPD) profile in nonhuman primates (NHPs) (cynomolgus monkeys).

Fab Mutant: T82(72)R, R94(84)S, S95(85)R; Aho numbering (actual numbering).

Fc Mutant: S(434)K (S440K in EU numbering).

### Example 4

### Production and Characterization of Low-Viscosity Mutants of GIPR (2G10.006) Antibody AQ Cloning, Expression, Purification and High Concentration Formulation of Low Viscosity Mutants of Antibody AQ

GIPR (2G10.006) AQ parent is described in U.S. Provisional Application 62/387,486 as 2G10_LC1.006 (SEQ ID NO: 74 of the cited patent application). The aforementioned US patent application is hereby incorporated by reference. Heavy chain mutant AQ (HC 1, 17, 85) with mutation sites Q1(1)E, R17(16)G, S85(75)A and light chain mutant AQ (LC 4 13 76 95 97 98) with mutation sites M4(4)L, V13(13)L, A76(60)D, S95(77)R, Q97(79)E, S98(80)P were produced as follows. Synthetic genes for GIPR (2G10.006)(antibody AQ) low viscosity mutants were produced, digested and ligated into plasmid expression vectors. Constructs were verified by DNA sequencing. Stable cell pools were created by electroporation of a clonal CHO host cell line. The pools were cultured under selection until viability reached greater than 85%. Pools were seeding into a fed-batch production culture for 10 days and centrifuged media was harvested.

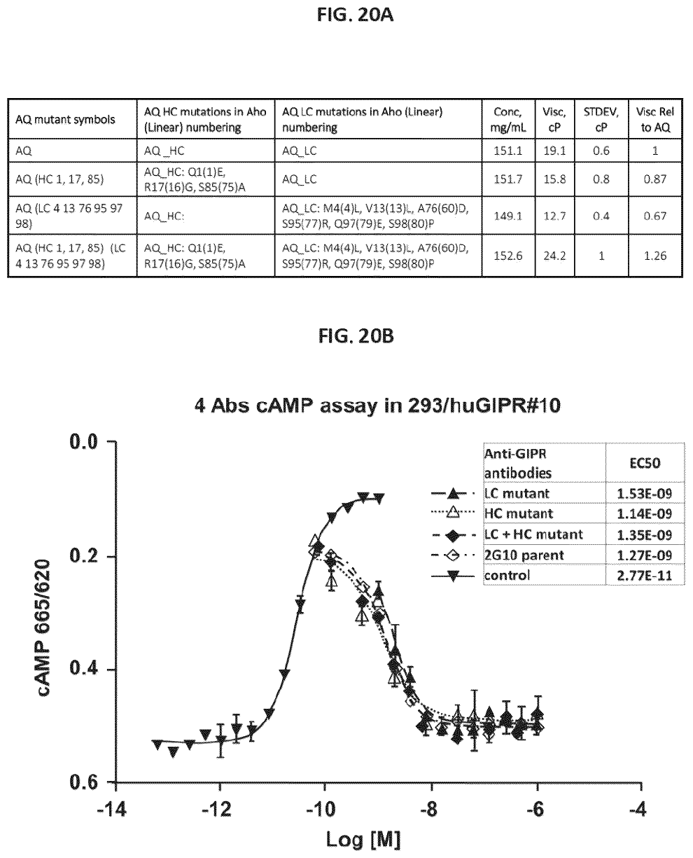

Harvested supernatants were sterile filtered and purified through a three column chromatography process consisting of Protein A, cation exchange, and anion exchange, similar to the process described earlier (Shukla et al. (2007), “Downstream processing of monoclonal antibodies—Application of platform approaches,” J. Chrom. B 848:28-39). The resulting purified pools were dialyzed into formulation buffer containing 20 mM acetate and 9% sucrose at pH 5.2 (without polysorbate), achieving a final pH of ˜5.2 and concentrated to approximately 150 mg/mL above 30 kDa cutoff filter via centrifugal ultrafiltration (FIG. 20A).

### Potency Measurements

Potency was measured by an assay utilizing mammalian cells 293/huGIPR expressing glucose-dependent insulinotropic polypeptide receptor (GIPR). The increasing concentrations of anti-GIPR parent AQ and the low-viscosity mutants were blocking the interaction of GIP with GIPR which induced cAMP changes monitored during the assay. An application of the assay was earlier described in Tseng C. C. et al. (1996), “Postprandial stimulation of insulin release by glucose-dependent insulinotropic polypeptide (GIP). Effect of a specific glucose-dependent insulinotropic polypeptide receptor antagonist in the rat,” J. Clin. Invest. 98:2440-2445.

### Viscosity Measurements

Viscosity analysis of AQ and two low viscosity mutants was performed on an Anton Paar Rheometer using a CP25-1/TG spindle. All measurements were performed at 25° C., controlled by a water bath attached to the sample cup. Viscosity measurements were collected manually with increasing shear rate from 0-2000 rpm. 10 viscosity measurement results at shear rate 1000 1/s and 10 viscosity measurement results at shear rate 2000 1/s were collected for each sample and averaged to report one viscosity value per sample.

It needs to be noted that precision of viscosity measurements is much better that accuracy, because the viscosity measurements are sensitive to even minor changes in some parameters, such as the state of the viscometer, temperature in the room and some other minor parameters at the time of the measurements. Therefore, it is important to measure all samples of interest in one setting or, if samples of interest are measured in two settings, have the same reference standard in both settings.

### Results

Anti-GIPR (2G10.006) antibody AQ belongs to high-viscosity germline subfamilies of heavy chain VH3|3-33 and light chain VK3|L16. Several mutations derived from FIGS. 11 and 12 were made in frames in the effort to reduce viscosity of AQ. The viscosity of the parent AQ and the two mutants measured in one viscometer setting revealed the following values: AQ-19.1 cP, AQ (HC 1, 17, 85)-15.8 cP, AQ (LC 4 13 76 95 97 98)-12.7 cP (FIG. 20). The heavy chain mutant AQ (HC 1, 17, 85) mutant was at 83% and the light chain mutant AQ (LC 4 13 76 95 97 98) was at 67% relative to the parent AQ. FIGS. 7 and 8 illustrate positions of the mutants on the viscosity versus pI plots for VH3 and VK3 family members. The in vitro cAMP activity was equally unaffected by viscosity mutations. The potency remained the same within the error margin of the in vitro cell-based assay (Sec FIG. 20B). To summarize, introduced mutations reduced viscosity without loss of the potency.

### Example 5

### GIPR Low Viscosity Mutant Light Chain V78F (LC V78F in Aho Numbering and LC V62F in Linear Numbering)

GIPR (2G10.006) antibody AQ showed a high viscosity of 23 cP at 150 mg/mL in A52Su formulation. This antibody featured a low-frequency residue V78 in Aho numbering (V62 in linear numbering) in the kappa light chain (LC V78 Aho). The frequency of occurrence of V78 is <1%, while F78 is >98% in light chain sequences related to the kappa germline. The residue LC V78 attracted attention, because it was a covariance violator. Covariance analysis allows establishing pair-wise conserved-residue positions based on the physiochemical properties of the residues in variable regions of antibodies, identifying incorrectly positioned residues (which are often non-germline residues). Covariance analysis further may suggest replacing the amino acids at the deviating positions with more common germline sequences that lead to a large conformational change uncovered by molecular-dynamics simulations (Kannan G., “Method of correlated mutational analysis to improve therapeutic antibodies,” U.S. Ser. No. 61/451,929, PCT/US 2012/028596, WO 2012/125495). In an effort to eliminate the covariance violation and increase the percentage of human sequences, the LC V78F mutation was introduced in the GIPR (2G10.006) antibody AQ.

Unexpectedly, viscosity of the mutant decreased by 25%, while maintaining similar potency for human GIPR as measured in cAMP (cell-based) assays. Both sequences, the GIPR (2G10.006) AQ parent and its LC V78F mutant are described in U.S. Provisional Application 62/387,486 as 2G10_LC1.006 (SEQ ID NO: 74 of the cited application) and 2G10_LC1.003 (SEQ ID NO: 71 of the cited application), respectively. The US patent application is hereby incorporated by reference. Newly discovered in the present invention is that such substitution resulted in about 25% reduction of viscosity by LC V78F mutation. Viscosity analysis of the GIPR_2G10.006 AQ and its V78F mutant was performed at 150 mg/ml in formulation containing 20 mM acetate, 9% sucrose at pH 5.2, 0.01% polysorbate 80, 1000 shear rate and 25 C using AR-G2 Magnetic Bearing Cone and Plate Rheometer from TA Instruments-Waters LLC. Cone plate size was 20 mm in diameter, 1.988° cone angle, equipped with Steel-990918 Peltier plate and operated using the Flow Sweep procedure. The measured viscosity values were 21 cP for GIPR_2G10.006 and 15.3 cP for GIPR (2G10.003) LC V78F mutant, which is 25% decrease in viscosity.

As noted in the previous example, precision of viscosity measurements is much better that accuracy. Viscosity at 150 mg/mL with 0.01% polysorbate is typically 10% lower than without polysorbate, which was observed in case of GIPR (2G10.006). Its viscosity was 23 cP without polysorbate (as for all 43 antibodies) and 21 cP with polysorbate.

### Example 6

### Cynomolgus Monkey Study

The antibodies designated as AK (control, also known as AMG 145 and evolocumab) and the Fab mutant, Fc mutant, and double mutant shown in Example 3 were generated using the methodology disclosed in Examples 1 and 2. The pharmacokinetic properties of these antibodies were tested in vivo by single subcutaneous bolus injection into male cynomolgus monkeys.

### Study Design

The study was conducted in male cynomolgus monkeys. The animals were 2.7 to 3.8 years old and weighted between 2.9 to 3.8 kg. The animals were acclimated to laboratory housing for 7 days before the initiation of dosing. Criteria for selection included acceptable results from the pretreatment cholesterol levels (including LDL and HDL) levels. Before the initiation of dosing, all animals were randomized and assigned to groups using a computer-based randomization procedure.

The test and control articles were administered subcutaneously into the mid-dorsal areas to the appropriate animals once on Day 1. The injection site(s) were shaved prior to administration and marked with indelible ink. The animals were temporarily restrained for dose administration and were not sedated. The dose volume for each animal was based on the most recent body weight measurement. For Groups 1 and 2, dose solutions were administered via 2 subcutaneous injections on the back of each animal (1 with test material, 1 with diluent). Injection sites were at least 5-6 cm apart. The test material was administered on the right of the spinal column for each animal. The diluent was delivered on the left of the spinal column for each animal. For Groups 3 and 4, dose solutions were administered via a single subcutaneous injection on the back of each animal. Dose levels and volumes for each group are summarized in FIG. 21.

Blood samples were collected by venipuncture into tubes containing Potassium (K2) EDTA at various time points over the duration of the in-life portion of this study (43 days). Animals were not fasted prior to serum chemistry blood collections.

Samples were chilled following blood collections, and split for preparation of cither serum or plasma. Samples were mixed gently and centrifuged. Blood samples were maintained on wet ice immediately after collection until centrifuged (1500-2000×g for approximately 10 minutes) at approximately 4° C. The resultant plasma or serum was separated and divided into 2 aliquots (primary and backup), transferred to appropriately labeled polypropylene tubes, and stored in a freezer set to maintain at −80° C. until analysis. Plasma samples were used to determine test article concentration for pharmacokinetic evaluation, serum samples were analyzed for cholesterol, HDL, and LDL.

### Pharmacokinetic Evaluation

Plasma samples were analyzed for concentration of each test antibody (antibody AK, AK Fab mutant, AK S440K Fc mutant, and AK Fab/S440K double mutant) using an enzyme-linked immunosorbent assay (ELISA). The assay uses recombinant human PCSK9 as the capture reagent and a horseradish peroxidase labeled antibody to human IgG1 as the detection reagent. Standards and quality control samples (QCs) are prepared by spiking antibody AK or low viscosity homologs into 100% cynomolgus monkey K2-EDTA pool. Costar 96-well microplate wells (Corning Incorporated) are coated with recombinant human PCSK9. After a blocking step, the standards, matrix blank (NSB), QCs (QCs) and test samples are loaded into the microplate wells after pre-treating with a dilution factor of 100 in Blocker™ BLOTTO in TBS (Thermo Scientific). The antibody AK in the samples is captured by the immobilized recombinant human PCSK9 coated on the microplate. Unbound material is removed by washing the microplate wells. Following washing, mouse anti-human IgG, Ab35, HRP conjugated detection antibody is added to the microplate wells to bind the captured antibody AK. Unbound detection antibody is removed by washing the microplate wells. A onc component TMB solution is added to the microplate wells for the detection of bound mouse anti-human IgG Ab35 HRP conjugate. The TMB substrate solution reacts with peroxide and, in the presence of HRP, creates a colorimetric signal that is proportional to the amount of antibody AK, or low viscosity mutant homolog, bound by the capture reagent. The color development is stopped using 2N sulfuric acid and the intensity of the color (optical density or OD) is measured at 450 nm minus 650 nm. Data are reduced using Watson version 7.4 SP3 (or later) data reduction package using a 4 parameter (Marquardt) regression model with a weighting factor of 1.

Pharmacokinetic parameters were estimated using WinNonlin pharmacokinetic software. A non-compartmental approach consistent with the subcutaneous route of administration was used for parameter estimation. All parameters were generated from individual concentrations in plasma from Day 1. The following parameters were determined
Tmax (the time after dosing at which the maximum observed concentration was observed),
Cmax (the maximum observed concentration measured after dosing),
AUC (0-t) (the area under the concentration versus time curve from the start of dose administration to the time after dosing at which the last observed quantifiable concentration using the linear or linear/log trapezoidal method),
AUC (0-t)/D (the AUC (0-t) divided by the dose administered), and
RAUC (the area under the curve from T1 and T2 at steady state divided by the area under the curve from T1 to T2 during the initial dosing interval).

### Results and Discussion

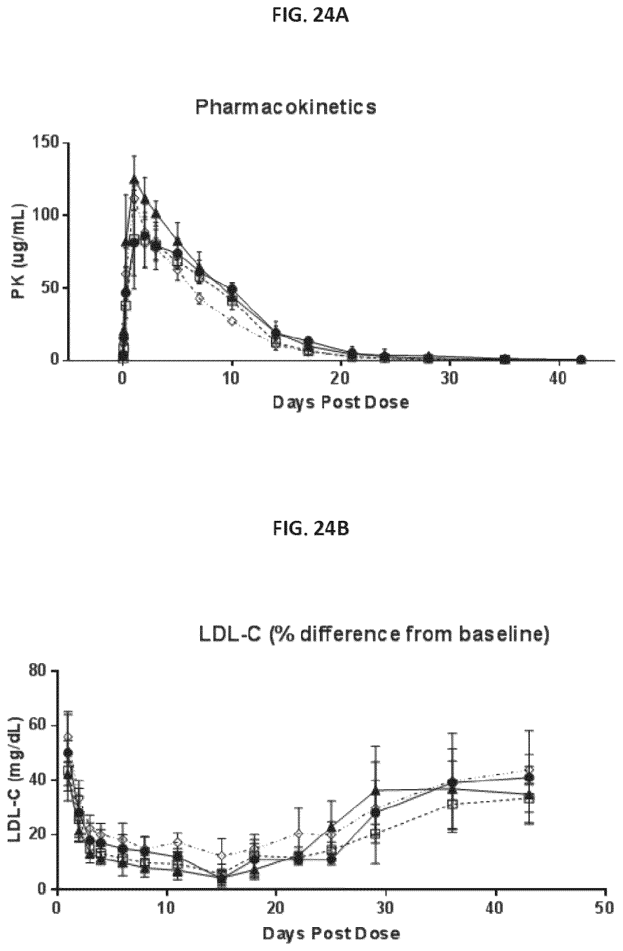

Test article concentrations plotted versus time are shown in FIGS. 24A and 24B (mean concentrations for each test article, n=4 at each time point). Pharmacokinetic parameters for the four test articles are summarized in FIG. 22. Antibodies containing the Fc mutation S440K (both antibody AK (Fc mutant) and antibody AK (Fc and Fab double mutant)) show a reduced Tmax (0.81 and 1 days respectively versus 2.5 days) and increased Cmax (125 and 112 μg/mL respectively versus 87.8 μg/mL) relative to antibody AK (control) and antibody AK (Fab mutant) indicating antibodies containing an Fc mutation that reduces viscosity are distributing more rapidly to the circulation following subcutaneous injection.

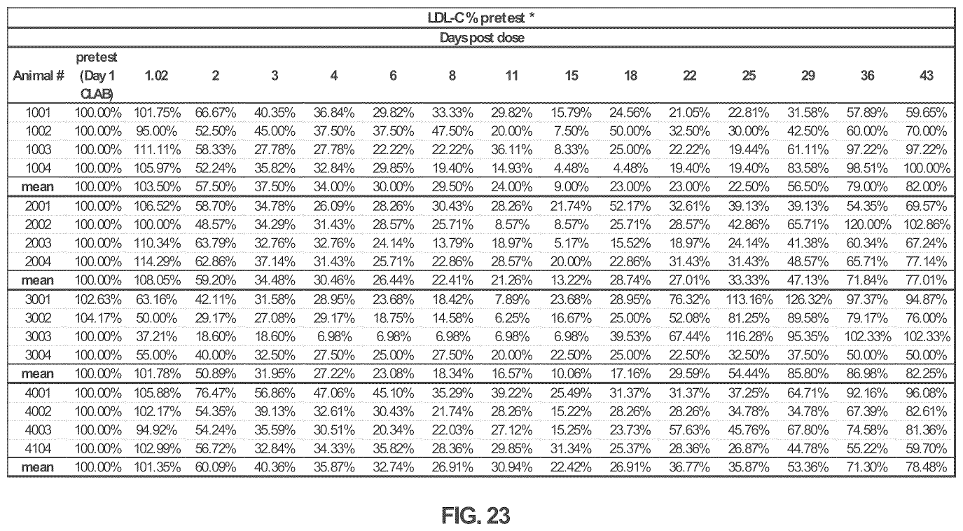

Administration of all low viscosity homologues of antibody AK resulted in expected pharmacologic mild to moderate decreases in low density lipoprotein (LDL) associated with decreased total cholesterol concentration compared to baseline (Day-6). The magnitude of decrease for total and low density lipoprotein cholesterol following administration of AK Fab mutant and AK S440K Fc mutant was generally similar to control animals with a trend toward recovery to baseline of AK S440K Fc mutant on Day 25 and AK (control) and AK Fab mutant on Day 29. The magnitude of decrease in total and low density lipoprotein cholesterol for AK Fab/S440K double mutant was generally less pronounced compared to control (antibody AK at 10 mg/kg). There were no changes in high density lipoprotein in any AK low viscosity homologue. Percentage changes in LDL-C relative to baseline are tabulated in FIG. 23 and are plotted versus time in FIGS. 24A and 24B.

### ABBREVIATIONS

Abbreviated terms used throughout this specification are defined as follows.
AEI allelic expression imbalance
ANOVA analysis of variance
AUC area under the curve
BSA bovine serum albumin
DMEM Dulbecco's Modified Eagle Medium
DMSO dimethyl sulfoxide
EDC 1-ethyl-3-[3-dimethylaminopropyl] carbodiimide hydrochloride
EDTA ethylenediaminetetraacetic acid
ELISA enzyme-linked immunosorbent assay
eQTL expression quantitative trait loci
ESI-TOF electrospray ionization time of flight
FACS fluorescence-activated cell sorting
FBS fetal bovine serum
FPLC fast protein liquid chromatography
FVB a strain of mice inbred for the Friend leukemia virus 1b (Fv1b) allele
H&E Hematoxylin and eosin
HA hypoxanthine
HIC hydrophobic interaction chromatography
HPLC high performance liquid chromatography
HRP horseradish peroxidase
HUVEC human umbilical vein epithelial cell
IBD inflammatory bowel disease
IDMEM DMEM without glutamine
IFN interferon
IL interleukin
MCP monocyte chemotactic protein
MSD macromolecular structure database
PBMC peripheral blood mononuclear cell
PBS phosphate-buffered saline
PCR polymerase chain reaction
PEG polyethylene glycol
PEI polyethylenimine
QTL quantitative trait loci
RPMI media developed at Roswell Park Memorial Institute
SNP single nucleotide polymorphism
TFA trifluoroacetic acid
TMB 3,3′,5,5′-Tetramethylbenzidine

1. A method of reducing the viscosity of an antigen binding protein, which comprises making one or more modifications in the sequence of the antigen binding protein selected from:
a. if the antigen binding protein comprises the VH3|3-33 germline subfamily, modifying the VH3 sequence to comprise one or more substitutions selected from 1X⁴, 17X⁵, and 85X⁶, wherein X⁴ is selected from D and E, X⁵ is selected from G, A, V, I, L, and M, and W, and X⁶ is selected from G.A, V, I, L, and M;
b. if the antigen binding protein comprises the VK3|L16 germline subfamily, modifying the VK3 sequence to comprise one or more substitutions selected from 4X¹⁰, 13X¹¹, 76X¹², 78F, 95X¹³, 97X¹⁴, and 98P, wherein X¹⁰ is selected from G, A, V, I, L, and M, X¹¹ is selected from G, A, V, I, L, and M, X¹² is selected from D and E, X¹³ is selected from R, K and H, and X¹⁴ is selected from D and E;
c. if the antigen binding protein comprises the VK3|L6 germline subfamily, modifying the VK3 sequence to comprise one or more substitutions selected from 76X¹² and 95X¹³;
d. modifying the Fc domain sequence to comprise one or more substitutions selected from 253X¹⁵, 440X¹⁶, and 439X¹⁷, wherein X¹⁵ is selected from G, A, V, I, L, and M, X¹⁶ is selected from R, K, and H, and X¹⁷ is selected from D and E, wherein the Fc domain sequence comprises only one of 440X¹⁶ and 439X¹⁷; and
e. modifying the C-terminus of the Fc domain sequence to comprise X¹⁸X¹⁹ wherein X¹⁸ is one to four amino acids selected from D and E or from H, K, and R, and X¹⁹ is selected from P, M, G, A, V, I, L, S, T, N, Q, F, Y and W and is absent when X¹⁸ comprises D or E, is present when X¹⁸ comprises K or R at its C-terminal end, and is present or absent when X¹⁸ comprises H at its C-terminal end;
wherein the amino acids in subparagraphs a, b, and c are numbered according to the Aho numbering system and the amino acids in subparagraph d are numbered according to the EU numbering system.

2.-8. (canceled)

9. The method of claim 1, which comprises modifying the VH3 sequence of an antigen binding protein comprising the VH3|3-33 germline subfamily to comprise one or more substitutions selected from 1E, 17G, and 85A.

10. The method of claim 9, which comprises modifying the VH3 sequence of an antigen binding protein comprising the VH3|3-33 germline subfamily to comprise substitutions 1E, 17G, and 85A.

11. The method of claim 1, which comprises modifying the VK3|L16 sequence of an antigen binding protein to comprise one or more substitutions selected from 4L, 13L, 76D, 78F, 95R, 97E, and 98P.

12. The method of claim 11, which comprises modifying the VK3|L16 sequence of an antigen binding protein to comprise substitutions 4L, 13L, 76D, 78F, 95R, 97E, and 98P.

13. The method of claim 1, which comprises modifying the VK3|L6 sequence of an antigen binding protein to comprise one or more substitutions selected from 76D and 95R.

14. The method of claim 13, which comprises modifying the VK3|L6 sequence of an antigen binding protein to comprise substitutions 76D and 95R.

15. The method of claim 1, which comprises modifying the Fc domain sequence to comprise one or more substitutions selected from 253A, 440K, and 439E.

16. The method of claim 15, which comprises modifying the Fc domain sequence to comprise substitutions 253A, 440K, and 439E.

17. The method of claim 1, which comprises modifying the C-terminus of the Fc domain to comprise an amino acid sequence selected from KP, KKP, KKKP, and E.

18.-19. (canceled)

20. An antigen binding protein which comprises one or more sequence modifications selected from:
a. a VH3|3-33 germline subfamily sequence comprising one or more substitutions selected from 1X⁴, 17X⁵, and 85X⁶, wherein X⁴ is selected from D and E, X⁵ is selected from G, A, V, I, L, and M, and X⁶ is selected from G.A, V, I, L, and M;
b. a VK3|L16 germline subfamily sequence comprising one or more substitutions selected from 4X¹⁰, 13X¹¹, 76X¹², 95X¹³, 97X¹⁴, and 98P, wherein X¹⁰ is selected from G, A, V, I, L, and M, X¹¹ is selected from G, A, V, I, L, and M, X¹² is selected from D and E, X¹³ is selected from R, K and H, and X¹⁴ is selected from D and E, wherein the antigen binding protein does not comprise only substitution 78F;
c. a VK3|L6 germline subfamily sequence comprising one or more substitutions selected from 76X¹² and 95X¹³;
d. an Fc domain sequence comprising one or more substitutions selected from 253X¹⁵, 440X¹⁶, and 439X¹⁷, wherein X¹⁵ is selected from G, A, V, I, L, and M, X¹⁶ is selected from R, K, and H, and X¹⁷ is selected from D and E, wherein the Fc domain sequence comprises only one of 440X¹⁶ and 439X¹⁷ and wherein the antigen binding protein comprises at least one of 253X¹⁵ or modifications selected from subparagraphs a, b, c, and e when X¹⁶ is K or X¹⁷ is E and the antigen binding protein specifically binds CD20; and
e. an Fc domain sequence comprising X¹⁸X¹⁹ at its C-terminus wherein X¹⁸ is one to four amino acids selected from D and E or from H, K, and R, and X¹⁹ is selected from P, M, G, A, V, I, L, S, T, N, Q, F, Y and W and is absent when X¹⁸ comprises D or E, is present when X¹⁸ comprises K or R at its C-terminal end, and is present or absent when X¹⁸ comprises H at its C-terminal end, and wherein the antigen binding protein comprises at least one of 253X¹⁵ or substitutions selected from subparagraphs a through e when PGKP, PGKKP, PGKKKP, or PGE appears at the C-terminus of the Fc domain and the antigen binding protein specifically binds CD20 or CD38;
wherein the amino acids in subparagraphs a, b, and c are numbered according to the Aho numbering system and the amino acids in subparagraph e are numbered according to the EU numbering system.

21.-27. (canceled)

28. The antigen binding protein of claim 20, wherein the VH3|3-33 germline subfamily sequence comprises one or more substitutions selected from 1E, 17G, and 85A.

29. The antigen binding protein of claim 28, wherein the VH3|3-33 germline subfamily sequence comprises substitutions 1E, 17G, and 85A.

30. The antigen binding protein of claim 20, wherein the VK3|L16 sequence comprises one or more substitutions selected from 4L, 13L, 76D, 78F, 95R, 97E, and 98P.

31. The antigen binding protein of claim 30, wherein the VK3|L16 sequence comprises substitutions 4L, 13L, 76D, 95R, 97E, and 98P.

32. The antigen binding protein of claim 20, wherein the VK3|L6 sequence comprises one or more substitutions selected from 76D and 95R.

33. The antigen binding protein of claim 32, wherein the VK3|L6 sequence comprises substitutions 76D and 95R.

34. (canceled)

35. The antigen binding protein of claim 20, comprising an Fc domain sequence comprising one or more substitutions selected from 253A, 440K, and 439E.

36.-41. (canceled)

42. The antigen binding protein of claim 20, which comprises at the C-terminus of the Fc domain an amino acid sequence selected from KP, KKP, KKKP, and E.

43.-54. (canceled)

55. The antigen binding protein of claim 20, wherein:
a. the antigen binding protein comprises the substitution 440X¹⁶ relative to a parental antibody lacking the 440X¹⁶ substitution,
b. the antigen binding protein reaches maximum serum concentration after subcutaneous injection faster than the parental antibody when the antigen binding protein and the parental antibody are administered at the same concentration, and
c. the antigen binding protein reaches a maximum serum concentration after subcutaneous injection that is higher than that of the parental antibody when the antigen binding protein and the parental antibody are administered at the same concentration.

56. The antigen binding protein of claim 55, wherein the antigen binding protein reaches maximum serum concentration at least about twice as fast as the parental antibody.

57. The antigen binding protein of claim 55, wherein the antigen binding protein reaches a maximum serum concentration that is at least about 25% higher than that of the parental antibody.

58.-59. (canceled)

60. The antigen binding protein of claim 55, wherein X¹⁶ is K.

61.-64. (canceled)

65. A method of preparing an antigen binding protein that reaches a higher maximum serum concentration than does a parental antibody and reaches the maximum serum concentration faster than does a parental antibody when both are administered at the same concentration, which comprises introducing sequence modification 440X¹⁶ in the parental antibody wherein X¹⁶ is selected from R, K, and H.

66. The method of claim 65, wherein the antigen binding protein reaches maximum serum concentration at least twice as fast as does the parental antibody.

67. The method of claim 65, wherein X¹⁶ is K.

68. (canceled)

69. The method of claim 67, wherein the parental antibody is a PCSK9 binding polypeptide.

71.-72. (canceled)

73. The method of claim 65, wherein the parental antigen binding protein is antibody AK from FIG. 1B (SEQ ID NOS: 134 and 136).

74.-78. (canceled)

79. A method of treating a PCSK9-related indication in a patient, which comprises administering the antigen binding protein of claim 55.

80. The method of claim 79, wherein the PCSK9-related indication is hypercholesterolemia.
